SEQUENCE LISTING (17 sequences)

SEQ ID NO: 1 - DNA, 5 bp, Artificial Sequence
  synthetic oligonucleotide
gagcg

SEQ ID NO: 2 - DNA, 5 bp, Artificial Sequence
  synthetic oligonucleotide
gacca

SEQ ID NO: 3 - DNA, 5 bp, Artificial Sequence
  synthetic oligonucleotide
ccaga

SEQ ID NO: 4 - DNA, 5 bp, Artificial Sequence
  synthetic oligonucleotide
agacc

SEQ ID NO: 5 - DNA, 12 bp, Artificial Sequence
  synthetic oligonucleotide
actgtgaatcgc

SEQ ID NO: 6 - DNA, 8 bp, Artificial Sequence
  synthetic oligonucleotide
  misc_feature (1)..(3): n comprises a universal nucleotide base (e.g., deoxyinosine)
  misc_feature (3)..(4): sulfur modification in place of the bridged oxygen of the phosphate backbone between nucleotides 3 and 4
nnntgact

SEQ ID NO: 7 - DNA, 8 bp, Artificial Sequence
  synthetic oligonucleotide
  misc_feature (1)..(3): n comprises a universal nucleotide base (e.g., deoxyinosine)
  misc_feature (3)..(4): sulfur modification in place of the bridged oxygen of the phosphate backbone between nucleotides 3 and 4
nnnagacc

SEQ ID NO: 8 - DNA, 8 bp, Artificial Sequence
  synthetic oligonucleotide
  misc_feature (1)..(3): n comprises a universal nucleotide base (e.g., deoxyinosine)
  misc_feature (3)..(4): sulfur modification in place of the bridged oxygen of the phosphate backbone between nucleotides 3 and 4
nnngacca

SEQ ID NO: 9 - DNA, 8 bp, Artificial Sequence
  synthetic oligonucleotide
  misc_feature (1)..(3): n comprises a universal nucleotide base (e.g., deoxyinosine)
  misc_feature (3)..(4): sulfur modification in place of the bridged oxygen of the phosphate backbone between nucleotides 3 and 4
nnngagcg

SEQ ID NO: 10 - DNA, 15 bp, Artificial Sequence
  synthetic oligonucleotide
tggtcggtctagtca

SEQ ID NO: 11 - DNA, 25 bp, Artificial Sequence
  synthetic oligonucleotide
tggtcggtctagtcacgctcggtct

SEQ ID NO: 12 - DNA, 13 bp, Artificial Sequence
  synthetic oligonucleotide
  misc_feature (1)..(3): n comprises a universal nucleotide base (e.g., deoxyinosine)
  misc_feature (3)..(4): sulfur modification in place of the bridged oxygen of the phosphate backbone between nucleotides 3 and 4
nnnagaccgacca

SEQ ID NO: 13 - DNA, 25 bp, Artificial Sequence
  synthetic oligonucleotide
agaccgagcgtgactagaccgacca

SEQ ID NO: 14 - DNA, 5 bp, Artificial Sequence
  synthetic oligonucleotide
gcgag

SEQ ID NO: 15 - DNA, 5 bp, Artificial Sequence
  synthetic oligonucleotide
accag

SEQ ID NO: 16 - DNA, 5 bp, Artificial Sequence
  synthetic oligonucleotide
ccaga

SEQ ID NO: 17 - DNA, 5 bp, Artificial Sequence
  synthetic oligonucleotide
tcagt

(19) United States
(12) Patent Application Publication
(10) Pub. No.: US 2023/0340457 A1
(43) Pub. Date: Oct. 26, 2023

(54) COMPOSITIONS, SETS, AND METHODS RELATED TO TARGET ANALYSIS

(71) Applicant: PRESIDENT AND FELLOWS OF HARVARD COLLEGE, Cambridge, MA (US)

(72) Inventors: Huy Quoc NGUYEN, Chestnut Hill, MA (US); Shyamtanu CHATTORAJ, Cambridge, MA (US); Chao-ting WU, Brookline, MA (US)

(73) Assignee: PRESIDENT AND FELLOWS OF HARVARD COLLEGE, Cambridge, MA (US)

(21) Appl. No.: 17/779,272

(22) PCT Filed: Nov. 24, 2020
(86) PCT No.: PCT/US20/61956

§ 371 Date: May 24, 2022

Related U.S. Application Data
(60) Provisional application No. 62/940,638, filed on Nov. 26, 2019.

(51) Int. Cl. C12N 15/10; C12Q 1/6816; C12Q 1/6841

(52) U.S. Cl. CPC C12N 15/1065; C12Q 1/6816; C12Q 1/6841

(57) ABSTRACT

The technology described herein is directed to compositions, sets, and methods for analyzing, detecting, and/or visualizing target molecules. In one aspect, described herein are sets of readout molecules to determine the identity of at least one oligonucleotide tag hybridized to at least one target molecule. In another aspect, described herein are methods of detecting said oligonucleotide tags bound to at least one target molecules using said set of readout molecules.

### CROSS-REFERENCE TO RELATED APPLICATIONS

This application claims benefit under 35 U.S.C. § 119(e) of U.S. Provisional Application No. 62/940,638 filed Nov. 26, 2019, the contents of which are incorporated herein by reference in their entirety.

### GOVERNMENT SUPPORT

This invention was made with government support HG008525 awarded by the National Institutes of Health. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Nov. 23, 2020, is named 002806-095230WOPT_SL.txt and is 5,053 bytes in size.

### TECHNICAL FIELD

The technology described herein relates to compositions, sets, and methods for analyzing, detecting, and/or visualizing target molecules.

### BACKGROUND

Replication, inheritance, and developmentally regulated gene expression are all genome-wide processes, occurring across all chromosomes simultaneously. Indeed, disruption of their genome-wide coordination can lead to genome damage, chromosome breakage and loss, aneuploidy, gross misexpression of genes, and disease. As such, there has been increasing demand for technologies that have the potential to query genomes in their entirety. Of these diverse methods, those providing information regarding the spatial organization of the genome are especially useful, as there is burgeoning evidence that the three-dimensional (3D) arrangement of chromosomes is strongly correlated with genome function and stability. Assays using proximity-based capture, such as Hi-C and other chromosome conformation capture technologies, as well as Genome Architecture Mapping (GAM) can report frequencies with which genomic regions interact and/or are found in the same subsection of the nucleus. These genome-wide methods have revealed the hierarchical manner by which whole genomes are organized, from cis interactions between enhancers and promoters to the intra- and inter-chromosomal compartmentalization of active and inactive chromatin.

However, methods for mapping genomes in situ are limited. Spatial genomics, where genomic loci are localized inside the 3D nucleus, is an emerging field concerned with the fact the spatial localization of DNA plays a critical role in how it is expressed, repaired, replicates, and functions. Current genome visualization methods are challenged by throughput as well as target detection, due to sequential labeling schemes and signal generation. As spatial genomics is limited by the ability of conventional microscopes to detect 4-5 colors at a time, a majority of techniques rely on the sequential visualization of targets. Such a method scales linearly and is not realistic for visualizing all ˜25,000 human genes. In order to provide spatial information as well as accommodate whole genomes, labeling techniques with an increased number of detectable targets and increased resolution are needed.

### SUMMARY

The technology described herein is directed at oligonucleotide compositions and sets, and corresponding methods for analyzing, detecting, and/or visualizing target molecules. In some embodiments of any of the aspects, the identity of at least one oligonucleotide tag (e.g., barcoded Oligopaints) bound to at least one target molecule is determined with a set of readout molecules. Such oligonucleotide tags and sets of readout molecules comprise a limited number of barcode regions and barcode-hybridizing regions, respectively. Compared to other methods (e.g., SOLiD chemistry), these readout molecule sets and sequencing methods, referred to herein as “Just Enough Barcodes” (JEB) or “Exact Barcodes”, are simplified, discard unnecessary oligos, and result in higher sequencing signal. Furthermore, the sets and methods as described herein demonstrate at least two advantages compared to other compositions and methods: (1) they decrease the number of oligonucleotide tags (e.g., Oligopaints) required to produce sufficient signal from a target molecule, and (2) they increase the number of barcode bits that can be detected, thus increasing the number of targets that can be uniquely identified. Ultimately, sets and methods as described herein permit the imaging of the entire human genome.

Described herein are applications of JEB barcodes to genome imaging and next-generation sequencing in order to reveal the complexity and biological importance of the genome's 3D configuration. JEB barcodes can be used with OligoFISSEQ, a method that leverages fluorescent in situ sequencing to simultaneously target, localize, and visualize any number of barcoded Oligopaint oligonucleotides that have been hybridized to the genome. Using OligoFISSEQ, 36 loci were spatially mapped across 6 chromosomes as well as 46 loci along the X chromosome in hundreds of individual human cells, achieving over >75% barcode recovery rate, producing high resolution spatial maps and chromosome traces. Such data demonstrate the ability of OligoFISSEQ and JEB barcodes to map every human gene with 8 rounds of sequencing. Visualization of entire genomes simultaneously in situ is essential to investigate mechanisms regulating genome organization and function. Targeting and visualizing numerous genomic targets in single cells, detailed 3D maps can be created using JEB barcodes in order to improve the understanding of the genome.

In one aspect described herein is a set of at least two readout molecules, each readout molecule comprising: (a) a 3′ barcode-hybridizing region of nucleotides or analogs thereof, the region comprising a unique sequence distinct from the 3′ region sequence of all other readout molecules in the set; (b) a 5′ non-barcode-hybridizing region of nucleotides or analogs thereof, the region comprising a sequence identical to the 5′ region sequence of all other readout molecules in the set; (c) a sulfur modification in place of the bridged oxygen of the phosphate backbone between the 5′ and 3′ regions; and (d) an optically detectable label.

In some embodiments of any of the aspects, the label is a fluorescent label.

In some embodiments of any of the aspects, the optically-detectable label comprises or further comprises biotin, amines, metals, metal nanoclusters, noble metal nanoparticles, anchoring molecules, quantum dots, acrydite, or DNA origami structures.

In some embodiments of any of the aspects, the label is located at the 5′ end of the readout molecule.

In some embodiments of any of the aspects, the set comprises four distinguishable labels.

In some embodiments of any of the aspects, the set comprises at least two distinguishable labels.

In some embodiments of any of the aspects, the set comprises at least three distinguishable labels.

In some embodiments of any of the aspects, the set comprises at least four distinguishable labels.

In some embodiments of any of the aspects, the readout molecules of each set which comprise a first 3′ region only comprise a first distinguishable label.

In some embodiments of any of the aspects, the readout molecules of each set which comprise any selected 3′ region only comprise a corresponding given distinguishable label.

In some embodiments of any of the aspects, the 3′ region is at least 1 nucleotide or analog thereof in length.

In some embodiments of any of the aspects, the 3′ region is 5 nucleotides or analogs thereof in length.

In some embodiments of any of the aspects, the 5′ region comprises only universal nucleotide bases.

In some embodiments of any of the aspects, the 5′ region comprises only deoxyinosine nucleotides.

In some embodiments of any of the aspects, the 5′ region is at least 1 nucleotide or analog thereof in length.

In some embodiments of any of the aspects, the 5′ region is 3 nucleotides or analogs thereof in length.

In some embodiments of any of the aspects, the at least two readout molecules comprise DNA and/or RNA.

In some embodiments of any of the aspects, the at least two readout molecules consist of or consist essentially of DNA and/or RNA.

In some embodiments of any of the aspects, the at least two readout molecules comprise a polypeptide.

In one aspect described herein is a set of at least two readout molecules, each readout molecule comprising: (a) a 3′ barcode-hybridizing region of nucleotides or analogs thereof, the region comprising a unique sequence distinct from the 3′ region sequence of all other readout molecules in the set; (b) a 5′ non-barcode-hybridizing region of nucleotides or analogs thereof; and (c) a sulfur modification in place of the bridged oxygen of the phosphate backbone between the 5′ and 3′ regions.

In some embodiments of any of the aspects, the 5′ non-barcode-hybridizing region of at least one readout molecule specifically hybridizes to an oligonucleotide.

In some embodiments of any of the aspects, the oligonucleotide comprises at least one detectable label.

In some embodiments of any of the aspects, the oligonucleotide specifically hybridizes to at least one other oligonucleotide.

In some embodiments of any of the aspects, the oligonucleotide is an amplification primer.

In some embodiments of any of the aspects, the oligonucleotide is a sequencing primer.

In some embodiments of any of the aspects, the oligonucleotide is an imager strand for super resolution microscopy.

In some embodiments of any of the aspects, the 5′ non-barcode-hybridizing region of at least one readout molecule is at least 5 nucleotides long.

In some embodiments of any of the aspects, the 5′ non-barcode-hybridizing region of at least one readout molecule is at least 10 nucleotides long.

In some embodiments of any of the aspects, the 5′ non-barcode-hybridizing region comprises a sequence identical to the 5′ region sequence of all other readout molecules in the set.

In some embodiments of any of the aspects, at least one readout molecule comprises an optically detectable label.

In some embodiments of any of the aspects, the label of at least one readout molecule is a fluorescent label.

In some embodiments of any of the aspects, the optically-detectable label comprises or further comprises a fluorophore, biotin, amines, metals, metal nanoclusters, noble metal nanoparticles, anchoring molecules, quantum dots, acrydite, or DNA origami structures.

In one aspect described herein is a readout molecule comprising: (a) a 3′ barcode-hybridizing region of nucleotides or analogs thereof, the region comprising a unique sequence distinct from the 3′ region sequence of all other readout molecules in a set of readout molecules; (b) a 5′ non-barcode-hybridizing region of nucleotides or analogs thereof, the region comprising a sequence identical to the 5′ region sequence of all other readout molecules in the set; (c) a sulfur modification in place of the bridged oxygen of the phosphate backbone between the 5′ and 3′ regions; (d) an optically detectable label; and (e) a nanoparticle.

In one aspect described herein is a readout molecule comprising: (a) a 3′ barcode-hybridizing region of nucleotides or analogs thereof, the region comprising a unique sequence distinct from the 3′ region sequence of all other readout molecules in a set of readout molecules; (b) a 5′ non-barcode-hybridizing region of nucleotides or analogs thereof, the region comprising a sequence identical to the 5′ region sequence of all other readout molecules in the set; (c) a sulfur modification in place of the bridged oxygen of the phosphate backbone between the 5′ and 3′ regions; and (d) a metal nanoparticle.

In one aspect described herein is a readout molecule comprising: (a) a 3′ barcode-hybridizing region of nucleotides or analogs thereof, (b) a 5′ non-barcode-hybridizing region of nucleotides or analogs thereof, (c) a sulfur modification in place of the bridged oxygen of the phosphate backbone between the 5′ and 3′ regions; and (d) a metal nanoparticle.

In some embodiments of any of the aspects, the readout molecule further comprises an optically detectable label.

In one aspect described herein is a set of at least two readout molecules, each readout molecule comprising: (a) a 3′ barcode-hybridizing region of nucleotides or analogs thereof, the region comprising a unique sequence distinct from the 3′ region sequence of all other readout molecules in the set; (b) a 5′ non-barcode-hybridizing region of nucleotides or analogs thereof, the region comprising a sequence identical to the 5′ region sequence of all other readout molecules in the set; (c) a sulfur modification in place of the bridged oxygen of the phosphate backbone between the 5′ and 3′ regions; and (d) an optically detectable label; wherein at least one readout molecule further comprises a nanoparticle.

In one aspect described herein is a set of at least two readout molecules, each readout molecule comprising: (a) a 3′ barcode-hybridizing region of nucleotides or analogs thereof, the region comprising a unique sequence distinct from the 3′ region sequence of all other readout molecules in the set; (b) a 5′ non-barcode-hybridizing region of nucleotides or analogs thereof, the region comprising a sequence identical to the 5′ region sequence of all other readout molecules in the set; and (c) a sulfur modification in place of the bridged oxygen of the phosphate backbone between the 5′ and 3′ regions; wherein at least one readout molecule further comprises a nanoparticle.

In some embodiments of any of the aspects, at least one readout molecule further comprises an optically detectable label.

In some embodiments of any of the aspects, the optically detectable label comprises a fluorophore.

In some embodiments of any of the aspects, the nanoparticle is linked to at least two readout molecules of the set.

In some embodiments of any of the aspects, the nanoparticle comprises a metal nanoparticle.

In some embodiments of any of the aspects, the metal nanoparticle is selected from the group consisting of Au, Ag, Ni, Co, Pt, Pd, Cu, Ti, and Al nanoparticles.

In some embodiments of any of the aspects, the nanoparticle comprises a gold nanoparticle.

In some embodiments of any of the aspects, the nanoparticle comprises a gold nanorod.

In some embodiments of any of the aspects, the nanoparticle has a diameter of about 1.2 nm.

In some embodiments of any of the aspects, the nanoparticle has a diameter of about 3 nm.

In some embodiments of any of the aspects, the nanoparticle has a diameter of about 5 nm.

In some embodiments of any of the aspects, the nanoparticle has a diameter of about 10 nm.

In some embodiments of any of the aspects, the nanoparticle has a diameter of about 30 nm.

In some embodiments of any of the aspects, the nanoparticle has a diameter of about 50 nm.

In some embodiments of any of the aspects, the nanoparticle is at the 3′ end of the readout molecule.

In some embodiments of any of the aspects, the nanoparticle is at least 20 nucleotides from the detectable label.

In some embodiments of any of the aspects, the nanoparticle is at least 30 nucleotides from the detectable label.

In one aspect described herein is use of a readout molecule or set thereof as described herein, for: (a) detection of at least one target molecule; (b) signal amplification; (c) branch reactions; (d) hybridization chain reaction (HCR); (e) signal amplification by exchange reaction (SABER); (f) rolling circle amplification (RCA); (g) in situ sequencing; (h) matrix attachment; or (i) super resolution microscopy.

In one aspect described herein is a method of detecting at least one target molecule in a sample, the method comprising: (a) contacting the sample with at least one oligonucleotide tag, each oligonucleotide tag comprising: (i) a recognition domain that binds specifically to a target molecule to be detected, and (ii) a street comprising a barcode region that comprises at least one barcode bit; (b) contacting the sample with a set of readout molecules as described herein; and (c) detecting the relative order of the optically detectable labels hybridized to the at least one oligonucleotide tag, wherein the at least one oligonucleotide tag is hybridized to the at least one target molecule, whereby the relative order of the optically detectable labels permits identification of which oligonucleotide tag is hybridized to the target molecule at that location.

In some embodiments of any of the aspects, the barcode region is unique to each oligonucleotide tag.

In some embodiments of any of the aspects, the total number of unique barcode bits is less than the total number of unique barcode bits possible.

In some embodiments of any of the aspects, the total number of unique barcode bits is less than 10% of the total number of unique barcode bits possible.

In some embodiments of any of the aspects, the total number of unique barcode bits is less than 1% of the total number of unique barcode bits possible.

In some embodiments of any of the aspects, the total number of unique barcode bits is at least 2 unique barcode bits.

In some embodiments of any of the aspects, the total number of unique barcode bits is no more than 10 unique barcode bits.

In some embodiments of any of the aspects, the barcode-hybridizing region is unique to each readout molecule.

In some embodiments of any of the aspects, the total number of unique barcode-hybridizing regions used in the set of readout molecules is less than the total number of unique barcode-hybridizing regions possible.

In some embodiments of any of the aspects, the total number of unique barcode-hybridizing regions in the set of readout molecules is less than 10% of the total number of unique barcode-hybridizing regions possible.

In some embodiments of any of the aspects, the total number of unique barcode-hybridizing regions in the set of readout molecules is less than 1% of the total number of unique barcode-hybridizing regions possible.

In some embodiments of any of the aspects, the total number of unique barcode-hybridizing regions in the set of readout molecules comprises at least 2 unique barcode-hybridizing regions.

In some embodiments of any of the aspects, the total number of unique barcode-hybridizing regions in the set of readout molecules comprises no more than 10 unique barcode-hybridizing regions.

In some embodiments of any of the aspects, the street further comprises a primer binding region for annealing a sequencing primer.

In some embodiments of any of the aspects, the detecting step is performed with a sequencing method.

In some embodiments of any of the aspects, the sequencing method comprises sequencing by ligation, sequencing by synthesis, sequencing by hybridization, and/or sequencing by cyclic reversible polymerization hybridization chain reaction.

In some embodiments of any of the aspects, sequencing by ligation comprises enzyme-based ligation.

In some embodiments of any of the aspects, sequencing by ligation comprises chemical ligation, copper assisted ligation, copper free click reaction, Amine-EDC based coupling, or thiol-maleimide Michael addition.

In some embodiments of any of the aspects, the specific hybridization of a readout molecule to a street is determined by the identity of the barcode region and barcode-hybridizing region.

In some embodiments of any of the aspects, the optically-detectable label is a fluorophore.

In some embodiments of any of the aspects, the detecting is performed with fluorescence microscopy.

In some embodiments of any of the aspects, the optically-detectable label further comprises biotin, amines, metals, metal nanoclusters, noble metal nanoparticles, anchoring molecules, quantum dots, acrydite, or DNA origami structures.

In some embodiments of any of the aspects, the detecting is performed with at least single cell resolution.

In some embodiments of any of the aspects, the detecting is performed with at least single nucleus resolution.

In some embodiments of any of the aspects, at least 2 target molecules are detected concurrently.

In some embodiments of any of the aspects, at least 3 target molecules are detected concurrently.

In some embodiments of any of the aspects, at least 10 target molecules are detected concurrently.

In some embodiments of any of the aspects, at least 20 target molecules are detected concurrently.

In some embodiments of any of the aspects, the target molecule comprises a nucleic acid, a polypeptide, a cell surface molecule, or an inorganic material.

In some embodiments of any of the aspects, the target molecule comprises DNA and/or RNA.

In some embodiments of any of the aspects, the target molecule comprises a polypeptide.

In some embodiments of any of the aspects, the target molecule is covalently or non-covalently linked to a nucleic acid, a polypeptide, a cell surface molecule, or an inorganic material.

In some embodiments of any of the aspects, the sample is a cell, cell culture, or tissue sample.

In some embodiments of any of the aspects, the sample comprises organoids.

In one aspect described herein is an enhanced method of detecting at least one target molecule in a sample, the method comprising: (a) contacting the sample with at least one oligonucleotide tag, each oligonucleotide tag comprising: (i) a recognition domain that binds specifically to a target molecule to be detected, and (ii) a street comprising a barcode region that comprises at least one barcode bit; (b) contacting the sample with a readout molecule or set thereof as described herein; and (c) detecting the relative order of the optically detectable labels hybridized to the at least one oligonucleotide tag, wherein the at least one oligonucleotide tag is hybridized to the at least one target molecule, whereby the relative order of the optically detectable labels permits identification of which oligonucleotide tag is hybridized to the target molecule at that location.

In some embodiments of any of the aspects, the signal of the optically detectable label of the at least one readout molecule comprising a nanoparticle is increased at least 1.5-fold compared to the signal of the optically detectable label of the same readout molecule not comprising the nanoparticle.

In some embodiments of any of the aspects, the signal of the optically detectable label of the at least one readout molecule comprising a nanoparticle is increased at least 3-fold compared to the signal of the optically detectable label of the same readout molecule not comprising the nanoparticle.

In some embodiments of any of the aspects, the signal of the optically detectable label of the at least one readout molecule comprising a nanoparticle is increased at least 10-fold compared to the signal of the optically detectable label of the same readout molecule not comprising the nanoparticle.

In some embodiments of any of the aspects, the signal of the optically detectable label of the at least one readout molecule comprising a nanoparticle is increased at least 50-fold compared to the signal of the optically detectable label of the same readout molecule not comprising the nanoparticle.

In some embodiments of any of the aspects, the sample comprises a human cell nucleus.

In some embodiments of any of the aspects, the sample comprises a nucleus from the cell of any organism.

In some embodiments of any of the aspects, the sample comprises metaphase chromosome spreads.

In some embodiments of any of the aspects, the metaphase chromosomes are obtained from a cultured cell nucleus.

In some embodiments of any of the aspects, the metaphase chromosomes are obtained from a nucleus extracted from a tissue section, an organoid, or a biopsy specimen.

In some embodiments of any of the aspects, the detectable labels are detected using electron microscopy, fluorescence microscopy, dark field microscopy, or any combination thereof.

In one aspect described herein is a method of karyotyping a biological sample, the method comprising: (a) contacting the sample with at least one oligonucleotide tag specific to at least one chromosome, each oligonucleotide tag comprising: (i) a recognition domain that binds specifically to a target molecule to be detected, and (ii) a street comprising a barcode region that comprises at least one barcode bit; (b) contacting the sample with a set of readout molecules as described herein; (c) detecting the relative order of the optically detectable labels hybridized to the at least one oligonucleotide tag, wherein the at least one oligonucleotide tag is hybridized to the at least one target molecule, whereby the relative order of the optically detectable labels permits identification of which oligonucleotide tag is hybridized to the target molecule at that location; and (d) determining the identity of at least one chromosome according to the identity of the least one oligonucleotide tag specific to the at least one chromosome.

In some embodiments of any of the aspects, the sample is contacted with at least one oligonucleotide tag specific to the p arm of the at least one chromosome.

In some embodiments of any of the aspects, the sample is contacted with at least one oligonucleotide tag specific to the q arm of the at least one chromosome.

In some embodiments of any of the aspects, the sample is contacted with at least one oligonucleotide tag specific to the p arm of the at least one chromosome, and at least one oligonucleotide tag specific to the q arm of the at least one chromosome.

In some embodiments of any of the aspects, the sample is contacted with at least two oligonucleotide tags specific to the p arm or the q arm of the at least one chromosome.

In some embodiments of any of the aspects, the sample is contacted with at least three oligonucleotide tags specific to the p arm or the q arm of the at least one chromosome.

In some embodiments of any of the aspects, the sample is contacted with at most 6 oligonucleotide tags specific to each chromosome arm.

In some embodiments of any of the aspects, the sample is contacted with at most 10 oligonucleotide tags specific to each chromosome arm.

In some embodiments of any of the aspects, the sample is contacted with at most 20 oligonucleotide tags specific to each chromosome arm.

In some embodiments of any of the aspects, the sample comprises a human cell nucleus.

In some embodiments of any of the aspects, the sample comprises a nucleus from the cell of any organism.

In some embodiments of any of the aspects, the sample comprises metaphase chromosome spreads.

In some embodiments of any of the aspects, the metaphase chromosomes are obtained from a cultured cell nucleus.

In some embodiments of any of the aspects, the metaphase chromosomes are obtained from a nucleus extracted from a tissue section, an organoid, or a biopsy specimen.

In one aspect described herein is a method of producing a high resolution image of at least one target molecule in a sample, the method comprising: (a) imaging the at least one target molecule using at least one round of a high resolution imaging method; and (b) determining the identity of the at least one imaged target molecule, comprising: (i) contacting the sample with at least one oligonucleotide tag, each oligonucleotide tag comprising: (A) a recognition domain that binds specifically to a target molecule to be detected, and (B) a street comprising a barcode region that comprises at least one barcode bit; (ii) contacting the sample with a set of readout molecules as described herein; and (iii) detecting the relative order of the optically detectable labels hybridized to the at least one oligonucleotide tag, wherein the at least one oligonucleotide tag is hybridized to the at least one target molecule, whereby the relative order of the optically detectable labels permits identification of which oligonucleotide tag is hybridized to the target molecule at that location.

In some embodiments of any of the aspects, the method comprises imaging at least 2 target molecules.

In some embodiments of any of the aspects, the method comprises imaging at least 12 target molecules.

In some embodiments of any of the aspects, the method comprises imaging at least 66 target molecules.

In some embodiments of any of the aspects, the method comprises imaging at least 258 target molecules.

In some embodiments of any of the aspects, the method comprises imaging at least 500 target molecules.

In some embodiments of any of the aspects, the method comprises imaging at least 5000 target molecules.

In some embodiments of any of the aspects, all of the target molecules are imaged at one time.

In some embodiments of any of the aspects, at least half of the target molecules are imaged at one time.

In some embodiments of any of the aspects, the method comprises at least two rounds of the high resolution imaging method.

In some embodiments of any of the aspects, the method comprises at least three rounds of the high resolution imaging method.

In some embodiments of any of the aspects, the method comprises at least five rounds of the high resolution imaging method.

In some embodiments of any of the aspects, the method comprises at least 20 rounds of the high resolution imaging method.

In some embodiments of any of the aspects, the high resolution imaging method is selected from the group consisting of: Oligo Stochastic Optical Reconstruction Microscopy (OligoSTORM); structured illumination microscopy (SIM); Stimulated emission depletion (STED) microscopy; and Oligo DNA point accumulation in nanoscale topology (DNA-PAINT).

In some embodiments of any of the aspects, the high resolution imaging method comprises Oligo Stochastic Optical Reconstruction Microscopy (OligoSTORM).

In some embodiments of any of the aspects, detecting the relative order of the optically detectable labels hybridized to the at least one oligonucleotide tag comprises at least 2 rounds of contacting the sample with the set of readout molecules.

In some embodiments of any of the aspects, detecting the relative order of the optically detectable labels hybridized to the at least one oligonucleotide tag comprises at least 3 rounds of contacting the sample with the set of readout molecules.

In some embodiments of any of the aspects, detecting the relative order of the optically detectable labels hybridized to the at least one oligonucleotide tag comprises at least 5 rounds of contacting the sample with the set of readout molecules.

In some embodiments of any of the aspects, detecting the relative order of the optically detectable labels hybridized to the at least one oligonucleotide tag comprises at least 10 rounds of contacting the sample with the set of readout molecules.

In some embodiments of any of the aspects, detecting the relative order of the optically detectable labels hybridized to the at least one oligonucleotide tag comprises at least 20 rounds of contacting the sample with the set of readout molecules.

In some embodiments of any of the aspects, the at least one target molecule comprises a 1 kb nucleic acid.

In some embodiments of any of the aspects, the at least one target molecule comprises a 15 kb nucleic acid.

In some embodiments of any of the aspects, the at least one target molecule comprises a 50 kb nucleic acid.

In some embodiments of any of the aspects, the at least one target molecule comprises a 100 kb nucleic acid.

In some embodiments of any of the aspects, the at least one target molecule comprises a 1 Mb nucleic acid.

In some embodiments of any of the aspects, the at least one target molecule comprises a chromosome.

In some embodiments of any of the aspects, the at least one target molecule comprises a genome.

### BRIEF DESCRIPTION OF THE DRAWINGS

FIG. 1A-1F is a series of schematics and images showing the OligoFISSEQ suite of methods. FIG. 1A shows a schematic of an Oligopaint oligo used for OligoFISSEQ. Portions of Ligation based Interrogation of Targets (LIT) primer site and barcode as well as Synthesis based Interrogation of Targets (SIT) primer site and barcode were used as Hybridization based Interrogation of Targets (HIT) bridge binding sites. FIG. 1B is a schematic showing an OligoFISSEQ workflow.

FIG. 1C is a schematic showing an LIT workflow. The LIT primer is phosphorylated (grey “P”). The first two nucleotides (nts) of each 8mer (i.e., 8-nt long nucleic acid) corresponds to a specific fluorophore, “N” denotes a mixture of A, C, T, or G, and “Z” denotes a universal base. The 8-mer hybridizes to a portion of the barcode region (e.g., SEQ ID NO: 5, ACTGTGAATCGC). FIG. 1D is a schematic showing an SIT workflow. FIG. 1E is a schematic showing a HIT workflow. FIG. 1F shows representative images of 4 rounds of OligoFISSEQ LIT, OligoFISSEQ SIT, and OligoFISSEQ HIT using a Chr19-20K library on PGP1f. Each image denotes the label for each round of OligoFISSEQ. Images are maximum intensity z-projection from multiple z slices. Images are from barcode specific fluorescent channels. First round of SIT is a combination of two channels: purple and green fluorescence (i.e., resulting in a white signal). Barcode detection rate with standard deviation per cell are shown for LIT, SIT, and HIT on the Chr19-20K library. Total cells for barcode detection rate per cell=79 for HIT, 85 for LIT and 66 for SIT from 4 technical replicates. Scale bar=10 μm.

FIG. 2A-2D is a series of schematics and images showing OligoFISSEQ-LIT on the 36plex-5K library. FIG. 2A is a schematic showing a layout of 36plex-5K library targets. Chromosome number is denoted by different labels. The schematic is not to scale. FIG. 2B is a series of images showing that 36plex-5K labels specific chromosomes. Top images show metaphase chromosome spreads from normal human male lymphoblasts hybridized with the 36plex-5K library. Bottom images show PGP1f cells hybridized with the 36plex-5K library. Labels correspond to chromosome code in FIG. 2A schematic. Oligopaints targeting a region on Chr 19 (Chr19-20K) also stained in the metaphase spreads (e.g., bright signal). Images are maximum z-projections. Scale bar=10 μm for all images. FIG. 2C shows images of a PGP1f nucleus (male) from four rounds of OligoFISSEQ-LIT off of Mainstreet and Backstreet sequencing of the 36plex-5K library. Images are from deconvolved, five-label merged maximum z-projections. FIG. 2D shows a 3-D representation of a field of view (FOV) containing three cells sequenced with four rounds of O-LIT. The largest cell in the FOV corresponds to the cell in FIG. 2C. Each round is represented on the z-axis, with the first round being closest to the nuclear DAPI outline (black). Maximum z-projection of sequencing signal from each round was taken, duplicated (2-images total for better visualization) and stacked on top of each other to form the image.

FIG. 3A-3F is a series of images, schematics, and graphs showing the every-pixel analysis pipeline on 36plex-5K. FIG. 3A is a schematic showing an every-pixel automated analysis pipeline. Various shades correspond to different chromosome targets. Zoomed in view (e.g., panel 2) shows a homolog of Chr2 (six targets) being decoded, mapped, and traced. FIG. 3B is a bar graph showing 36plex-5K Mainstreet-Backstreet (MSBS) target detection after Tier 2. 80.2±7.3% of targets are detected in 638 cells across 13 replicates. Cartoon chromosomes on x-axis denote target chromosome. Note that 3qR3 and 5pR3 targets share the same barcode and are not included. FIG. 3C is an image showing chromosome traces of panel a nucleus (from FIG. 2A-2D) using targets decoded after Tier 2. 64/66 (97%) 36plex-5K targets were detected. Image is from the first round of LIT with target identities overlaid. Different lines show chromosome traces between detected targets. FIG. 3D is a 3-D representation of the nucleus in FIG. 3B-3C. Chromosome targets are colored as shown. Black spheres are undetected targets. FIG. 3E is an image showing a single-cell pairwise spatial distance matrix after Tier 2 detection of the nucleus in FIG. 3B-3C. Targets are represented on the x-axis with each homolog separated. Undetected targets are represented by grey lines. FIG. 3F is an image showing 36plex-5K population pairwise spatial distance measurements. Average pairwise spatial distance from cell population (n=638 cells) are shown after Tier 1 detection. Measurements from homologous targets were combined.

FIG. 4A-4D is a series of images, schematics, and graphs showing OligoFISSEQ-eLIT. FIG. 4A shows a schematic for just enough barcode (JEB) technology used with OligoFISSEQ-exact barcode Ligation based Interrogation of Targets (eLIT). JEB labeled 8-mers detailed in grey box. The 3′ end of each 8-mer is completely complementary to the 5-nt eLIT barcode bit. “I” on 5′ end can be deoxyinosines (“universal base”). As shown in FIG. 4A, the JEB oligos can include (shown 5′ to 3′): nnnTGACT (SEQ ID NO: 6), nnnAGACC (SEQ ID NO: 7), nnnGACCA (SEQ ID NO: 8), and/or nnnGAGCG (SEQ ID NO: 9), wherein “n” comprises a universal nucleotide base (e.g., deoxyinosine), and wherein the oligo further comprises a sulfur modification in place of the bridged oxygen of the phosphate backbone between nucleotides 3 and 4. JEB oligos reduces the pool of labeled 8mers to four. FIG. 4B shows a schematic for OligoFISSEQ-eLIT with JEB. JEB oligos (see e.g., FIG. 4A) share complete complementarity (5-nt) with eLIT barcode bits. FIG. 4B shows JEB oligos (e.g., SEQ ID NO: 8 or 7) hybridizing with a portion of the barcode region (e.g., SEQ ID NO: 10, TGGTCGGTCTAGTCA). FIG. 4C shows a series of images of 36plex-1K library sequenced five rounds with OligoFISSEQ-eLIT PGP1f cell. Top image shows cropped field of view of cells after 1st round of sequencing. Bottom panels show zoomed in nucleus (square in top image) sequenced five rounds with a “toto hybe” detecting all targets with a labeled secondary oligo (T). Extranuclear puncta are fiducial tetraspeck beads. Images are deconvolved maximum z-projections. Scale bar=10 um. FIG. 4D is a bar graph showing target detection efficiency of 36plex-1K library after Tier 2 detection and five rounds of O-LIT with SOLiD reagents (light grey) or eLIT with JEB (dark grey). Average detection: SOLiD=54.6% (n=41 cells from 1 replicate), JEB=76.4±8.6% (n=439 cells from 8 replicates). Error bars=population standard deviation.

FIG. 5A-5F is a series of images, schematics, and graphs showing the tracing of 46 regions along Chromosome X. FIG. 5A is a layout of a ChrX-46plex-2K library overlaid onto the cropped field of view from the first round of LIT sequencing in PGP1f. Micrograph is a deconvolved maximum intensity z-projection. FIG. 5B shows images of a zoomed-in cell (square from the field of view FIG. 5A) with the ChrX-46plex-2K library hybridized and sequenced with five rounds off of the Mainstreet and Backstreet with OligoFISSEQ-eLIT. White numbers denote sequencing round. Left panel shows view of the entire nucleus (DAPI) with 1st round sequencing, Smaller panels to the right show zoomed-in view of each sequencing round with a “toto hybe” detecting all targets with a labeled secondary oligo (T). Images are deconvolved maximum z-projection. FIG. 5C is a bar graph showing target detection efficiency of the ChrX-46plex-2K library after Tier 2 detection and five rounds of eLIT (MS and MSBS) in PGP1f. Average detection=74.6±2.5% (n=146 cells from 5 replicates). Error bars=population standard deviation. FIG. 5D-5E are a series of images showing ChrX-46plex-2K mapping and tracing (FIG. 5D) and 3-D visualization (FIG. 5E) after interpolation of the cell from FIG. 5B. FIG. 5F is an image showing a pairwise distance matrix after interpolation of nucleus from FIG. 5B. FIG. 5G is an image showing ChrX-46plex (MS and MSBS) population pairwise spatial distance measurements. Average pairwise spatial distance matrix from cell population (n=61 cells from 2 replicates) after Tier 1 detection.

FIG. 6A-6D is a series of images, schematics, and graphs showing OligoFISSEQ extensions and applications. FIG. 6A is an image showing OligoFISSEQ detection of single gene targets and a schematic of gene targets. The sample field of view is from the 1st round of O-eLIT on PGP1f. Squares outline specific gene targets after 5 rounds. Number reflects percentage of targets detected, out of 11. The image is a deconvolved maximum z-projection. FIG. 6B is a bar graph showing target detection efficiency from a 6-gene library after Tier 2 detection and five rounds of 0-eLIT off of Mainstreet and Backstreet. n=61 cells from 2 replicates. Error bars=Standard Deviation among cell population. FIG. 6C is an image combining OligoFISSEQ-LIT and immunofluorescence. The 36plex-5K library was sequenced for four rounds with OligoFISSEQ-LIT followed by immunofluorescence. Image is a maximum intensity z projection with chromosome traces overlaid.

FIG. 6D is a series of images showing the combination of OligoSTORM and OligoFISSEQ-LIT (0-LITSTORM) in order to multiplex genome visualization with super-resolution microscopy. The Chr2-6plex-5K library was hybridized to PGP1f cells and prepared for 1 round of OligoSTORM to visualize all targets simultaneously, followed by 2 rounds of O-LIT to decode targets. Left panel shows OligoSTORM image after DBSCAN, with identity of clusters unknown at this time point. Middle panel shows a micrograph from the 1st round of O-LIT. Image is a deconvolved maximum z-projection. Right middle panel shows a magnified view of targets decoded. Right panel shows a magnified view of targets from OligoSTORM. Bottom panels display the diffraction-limited and STORM images for each target.

FIG. 7 is a workflow chart of the chromosome tracing process.

FIG. 8 is a series of histograms of distances between consecutive loci 36plex.

FIG. 9 is a histogram of distances between consecutive loci ChrX-46plex.

FIG. 10A-10H is a series of schematics, images, and graphs showing methods for highly multiplexed in-situ visualization and identification of targets. FIG. 10A is a schematic of oligonucleotide modifications to permit specific cleavage. As a non-limiting example, shown here is a phosphorothiolate modified oligonucleotide. A sulfide modification replaces the bridged oxygen in the oligonucleotide phosphate backbone, denoted by the asterisk (*). Treatment with heavy metals under mild conditions (50 mM AgNO₃) results in cleavage of the oligonucleotide at the sulfide substitution (denoted by the scissors symbol), resulting in the oligonucleotide being separated into two parts, and generation of 5′ phosphate (PO₄). Note that this sulfide modification can be placed at any nucleotide position internal to the oligonucleotide, demonstrating its flexibility. FIG. 10B is a schematic showing an exemplary use of the phosphorothiolate oligo for enzyme-mediated ligation, targeted cleavage, and oligonucleotide extension. A primer comprising a 5′ phosphate is extended by ligation with the phosphorothiolate oligo. AgNO₃ mediated cleavage occurs at a user-specified position (marked with *), resulting in cleavage of the extended oligo and the regeneration of a 5′ PO₄. The oligo can be extended further by introduction of another sulfide modified oligo. FIG. 10C is a schematic showing use of phosphorothiolate oligo chemistry to improve fluorescent in situ sequencing (FISSEQ) specificity and signal. The schematic shows the four label sequencing by ligation (SBL) scheme used by SOLiD (Sequencing by Oligonucleotide Ligation and Detection). As shown herein, 8-nt (nucleotide) fluorescently labeled oligos can be used. SOLiD uses a di-base scheme where, from the 3′ to 5′ end: nt 1 and 2 correspond to a specific fluorophore (shaded circles in table); nt 3 to 5 are a mixture of A, C, T, and G; and nt 6 to 8 are universal bases, with a phosphorothiolate between nt 5 and 6 (denoted by *). The pool of SOLiD oligos consists of 1,024 different oligo species. Comparatively, the “Just Enough Barcodes” (JEB) scheme and chemistry described herein decreases the number of oligo species, for example to 4, by restricting nt 1 to 5 to a specific sequence and having nt 6-8 as deoxyinosine nucleotides (“universal bases”). Note that the 5 nt sequence restriction in JEB is a non-limiting example application. Phosphorothiolate modified oligos are amenable to any combination of designated/restricted nt. Also, fluorophore conjugation is flexible and compatible with many different fluorophores or detectable labels. FIG. 10D is a schematic showing use of JEB chemistry to improve OligoFISSEQ. Depicted herein is a barcoded Oligopaint compatible with SBL. FIG. 10E is a schematic of OligoFISSEQ using JEB. First, the 5′ phosphorylated ligation primer hybridizes to the ligation sequencing primer binding site on the Oligopaint street. Next, JEB oligos are flowed in with DNA ligase. JEB oligos (e.g., SEQ ID NO: 6-9) with complementarity to the barcode (e.g., SEQ ID NO: 11, TGGTCGGTCTAGTCACGCTCGGTCT) hybridize and ligate. Non-ligated JEBs are washed out, and an image is captured. The JEB oligo is cleaved between nt 5 and 6, releasing fluorophore and exposing the 5′ PO₄. The cycle repeats until the entire barcode is sequenced. Exemplary portions of the ligated oligos include SEQ ID NO: 2 (GACCA), SEQ ID NO: 12 (nnnAGACCGACCA, wherein n comprises a universal nucleotide base (e.g., deoxyinosine), and wherein the oligo further comprises a sulfur modification in place of the bridged oxygen of the phosphate backbone between nucleotides 3 and 4) or SEQ ID NO: 13 (AGACCGAGCGTGACTAGACCGACCA). FIG. 10F is a series of images showing that OligoFISSEQ with JEB oligos had improved signal over SOLiD oligos. Shown herein are representative images of the first round of sequencing 9,000 barcoded Oligopaints targeting a 2.4 Mb region on Chr19 (chromosome 19). Two nuclei were present in each image. Imaging conditions (e.g., exposure, excitation, etc.) were the same for both images. FIG. 10G is a bar graph showing that OligoFISSEQ with JEB oligos showed a greater than four-times-higher signal to noise ratio (SNR) compared to SOLiD oligos. The histogram compares SNR of sequencing signals from images in FIG. 10F. SNR was calculated by measuring brightest pixel in each sequencing focus from the z-projection of FIG. 10F images and dividing by the average nuclear background intensity (i.e. the area where a sequencing signal is not present). Values were then normalized to SNR with SOLiD. FIG. 10H is a schematic showing a workflow for multiplexed genome visualization. A primary Oligopaint library (small grey circles) is hybridized to fixed cells. Each target can then be sequenced over multiple rounds (e.g., 3 as shown herein). After sequencing is finished, each focus can be decoded, compared to the key, and the specific barcode identified.

FIG. 11A-11E is a series of schematics and images showing “Just Enough Barcodes” (JEB) overhangs. FIG. 11A shows a schematic of the Chr2-6plex-1K library color barcodes. 6 genomic regions (denoted A-F in FIG. 11A) were targeted along human chromosome 2 by 1,000 Oligopaint oligos per genomic region. Each genomic region contains a specific barcode; here (e.g., in FIG. 11A-11E) one round is represented. FIG. 11B shows one round of OligoFISSEQ-eLIT with JEBs visualizing Chr2-6plex-1K library in PGP1f cells. One of the JEBs (indicated with arrows) contains an oligonucleotide (oligo) overhang instead of a fluorophore and is not visualized in the images (e.g., Cy3). Images show maximum intensity z-projection. Scale bar is 10 μm. FIG. 11C shows the same nucleus as in FIG. 11B after hybridization of fluorophore labeled oligo complementary to the oligo overhang on the JEB. Cy3 signal appears. In FIG. 11D, the fluorophore-labeled oligo hybridized to the JEB overhang is washed off with 60% formamide. The Cy3 signal disappears. In FIG. 11E, a new dual fluorophore labeled oligo is hybridized to the JEB overhang. Cy3 signal reappears.

FIG. 12A-12B is a series of images and graphs showing 129plex Oligopaint FISH on Metaphase spreads from human male peripheral lymphocytes.

FIG. 13 is a series of images showing 4 rounds of OligoFISSEQ on human male peripheral lymphocytes using JEB oligos.

FIG. 14 is an image showing decoding using 3 rounds of OligoFISSEQ on Metaphase spreads from human male peripheral lymphocytes.

FIG. 15 is an image showing karyotyping using 4 rounds of OligoFISSEQ on Metaphase spreads from human male peripheral lymphocytes.

FIG. 16 is a series of images and graphs showing fluorescence signal enhancement of in situ sequencing signal by gold nano-particles (Au-NPs). Main street and back street sequencing primers were labeled with 50 nm Au-NP and 30 nm Au-NP. The targets are 6 spots in Chr 19: ˜0.5 kb, 15 oligos per spot. TxR indicates “Texas Red” fluorophore.

FIG. 17A-17B is a series of images and graphs showing the combination of OligoSTORM and OligoFISSEQ to accelerate genome super-resolution imaging. In FIG. 17A, the 36plex-5K library was hybridized to PGP1f cells and imaged with 1 round of OligoSTORM (2 hours) to visualize all 66 targets simultaneously, followed by 4 rounds of OligoFISSEQ (2-3 hours per round) to decode targets. FIG. 17B shows each chromosomal region imaged with OligoSTORM displayed separately; orientation may differ from that in FIG. 17A.

### DETAILED DESCRIPTION

Embodiments of the technology described herein include oligonucleotide compositions and sets, and corresponding methods for analyzing, detecting, and/or visualizing target molecules. In some embodiments of any of the aspects, the identity of at least one oligonucleotide tag (e.g., barcoded Oligopaints) bound to at least one target molecule is determined with a set of readout molecules. Such oligonucleotide tags and sets of readout molecules comprise a limited number of barcode regions and barcode-hybridizing regions, respectively. Compared to other methods (e.g., SOLiD chemistry), these readout molecule sets and sequencing methods, referred to herein as “Just Enough Barcodes” (JEB) or “Exact Barcodes”, are simplified, discard unnecessary oligos, and result in higher sequencing signal. Furthermore, the sets and methods as described herein demonstrate at least two advantages compared to other compositions and methods: (1) they decrease the number of oligonucleotide tags (e.g., Oligopaints) required to produce sufficient signal from a target molecule, and (2) they increase the number of barcode bits that can be detected, thus increasing the number of targets that can be uniquely identified. Ultimately, sets and methods as described herein permit the imaging of the entire human genome.

Accordingly, in one aspect described herein is a set of at least two readout molecules, each readout molecule comprising: (a) a 3′ barcode-hybridizing region of nucleotides or analogs thereof comprising a unique sequence distinct from the 3′ region sequence of all other readout molecules in the set; (b) a 5′ non-barcode-hybridizing region of nucleotides or analogs thereof comprising a sequence identical to the 5′ region sequence of all other readout molecules in the set; (c) a sulfur modification in place of the bridged oxygen of the phosphate backbone between the 5′ and 3′ regions; and (d) an optically detectable label.

In another aspect described herein is a method of detecting (and/or analyzing) at least one target molecule in a sample, the method comprising: (a) contacting the sample with at least one oligonucleotide tag, each oligonucleotide tag comprising: (i) a recognition domain that binds specifically to a target molecule to be detected (and/or analyzed), and (ii) a street comprising a barcode region that comprises at least one barcode bit; (b) contacting the sample with a set of readout molecules as described herein; and (c) detecting the relative order of the optically detectable labels hybridized to the at least one oligonucleotide tag, wherein the at least one oligonucleotide tag is hybridized to the at least one target molecule, whereby the relative order of the optically detectable labels permits identification of which oligonucleotide tag is hybridized to the target molecule at that location.

As used herein, the term “readout molecule” refers to a molecule comprising at least 1) a detectable label and 2) a barcode-hybridizing region, which refers to an oligonucleotide sequence that is complementary to at least a portion of at least one oligonucleotide tag and/or hybridizes specifically with at least a portion of at least one oligonucleotide tag. As used herein, the term “oligonucleotide tag” is an oligonucleotide that comprises a recognition domain and/or at least one street. The recognition domain binds specifically to a target molecule to be detected, and the street comprises a barcode region that comprises at least one barcode bit (or unit). Each barcode-hybridizing region hybridizes to a specific barcode bit of the oligonucleotide tag. In some embodiments of any of the aspects, the total number of unique barcode bits and/or barcode-hybridizing regions is significantly less than the total number of unique barcode bits bit possible. Such a limited number decreases the total number of readout molecules needed and increases the signal-to-noise-ratio of the readout molecules during detection.

Described herein are readout molecules and sets thereof. As used herein, the term “readout molecule” refers to a molecule comprising at least 1) a detectable label and 2) an oligonucleotide sequence that is complementary to at least a portion of at least one oligonucleotide tag and/or hybridizes specifically with at least a portion of at least one oligonucleotide tag. In some embodiments of any of the aspects, the readout molecule comprises a barcode-hybridizing region that is complementary to at least a portion (e.g., the barcode region) of at least one oligonucleotide tag and that hybridizes to at least a portion of the oligonucleotide tag. In some embodiments of any of the aspects, a readout molecule comprises one barcode-hybridizing region and at least one other region (e.g., a non-barcode-hybridizing region). In some embodiments of any of the aspects, a readout molecule comprises one barcode-hybridizing region and at least one modification. In some embodiments of any of the aspects, a readout molecule comprises a single barcode-hybridizing region. In some embodiments of any of the aspects, a readout molecule comprises a single label, or a single optically-detectable label, or a single label that provides a single optically-detectable signal.

In some embodiments of any of the aspects, the readout molecule is DNA and/or RNA. In some embodiments of any of the aspects, the readout molecule comprises DNA and/or RNA. In some embodiments of any of the aspects, the readout molecule consists of or consists essentially of DNA and/or RNA. In some embodiments of any of the aspects, the readout molecule comprises a polypeptide.

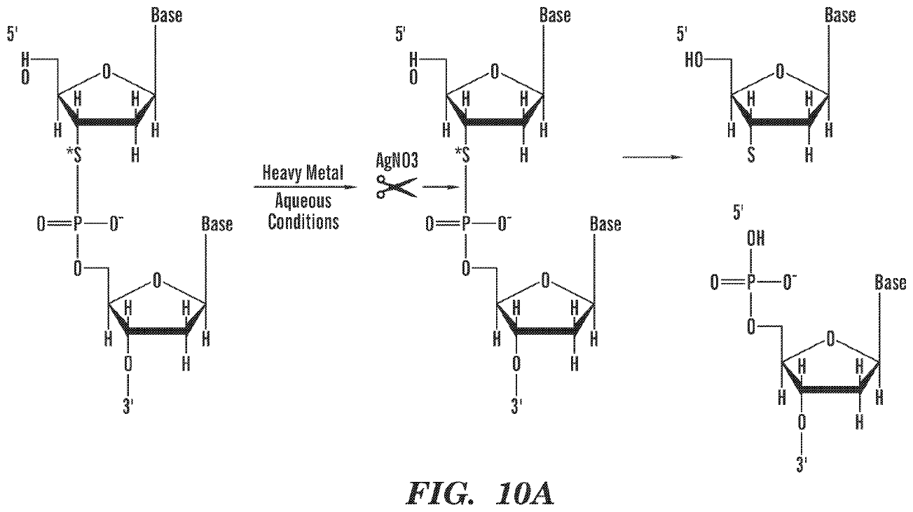

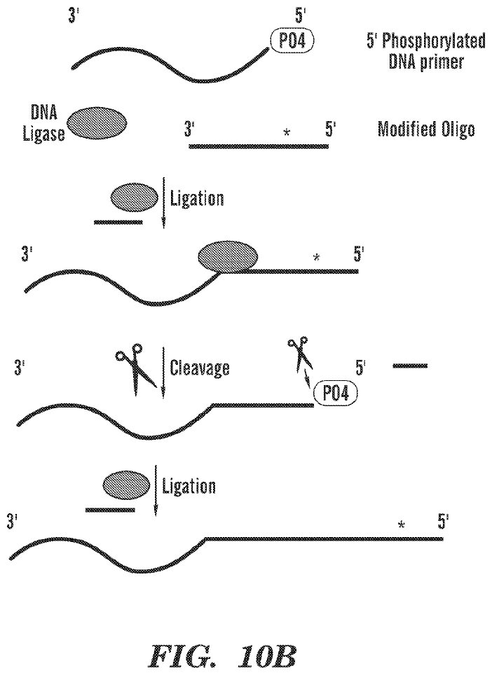

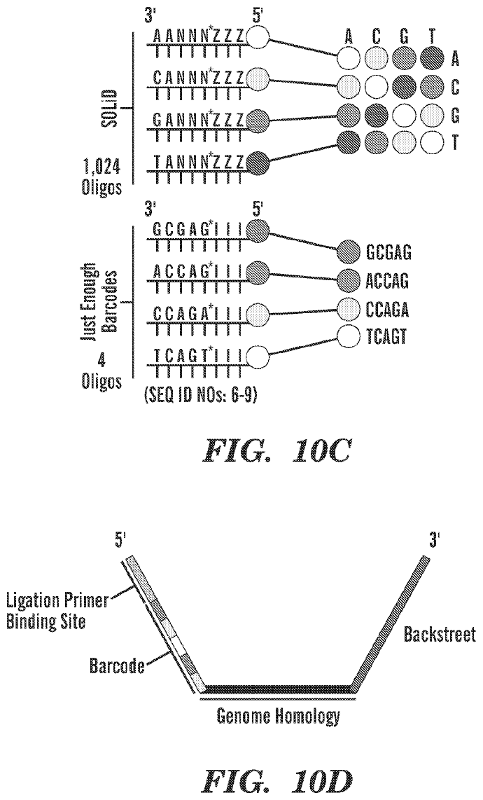

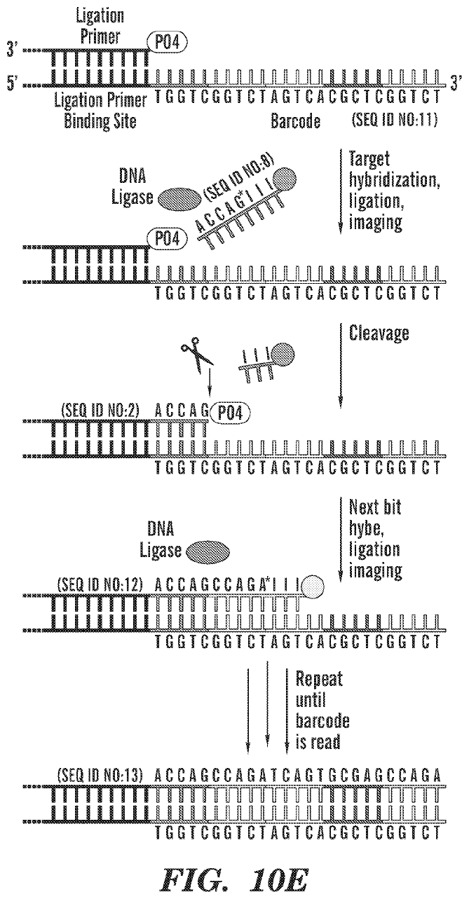

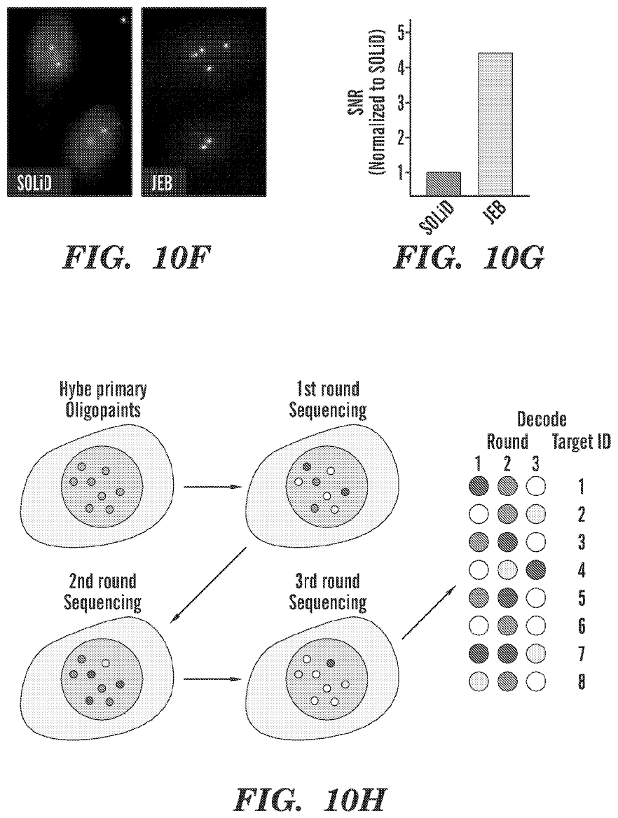

In some embodiments of any of the aspects, the readout molecule comprises: (a) a 3′ barcode-hybridizing region, (b) a 5′ non-barcode-hybridizing region, (c) a modification between the 3′ region and 5′ region, and (d) a detectable label (see e.g., FIG. 10C). In some embodiments of any of the aspects, the readout molecule comprises: (a) a 5′ barcode-hybridizing region, (b) a 3′ non-hybridizing region, (c) a modification between the 3′ region and 5′ region, and (d) a detectable label. In some embodiments of any of the aspects, the readout molecule comprises: (a) a barcode-hybridizing region, (b) at least one non-barcode-hybridizing region, (c) a modification between the barcode-hybridizing region and the at least one non-barcode-hybridizing region, and (d) a detectable label.

In some embodiments of any of the aspects, the readout molecule comprises: (a) a 3′ barcode-hybridizing region of nucleotides or analogs thereof comprising a unique sequence distinct from the 3′ region sequence of all other readout molecules in the set; (b) a 5′ region of nucleotides or analogs thereof comprising a sequence identical to the 5′ region sequence of all other readout molecules in the set; (c) a sulfur modification in place of the bridged oxygen of the phosphate backbone between the 5′ and 3′ regions; and (d) an optically detectable label (see e.g., FIG. 10C).

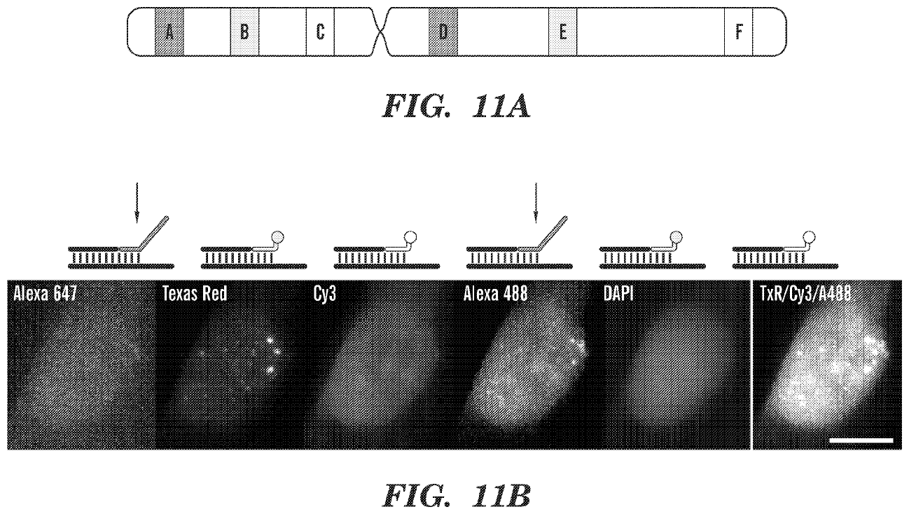

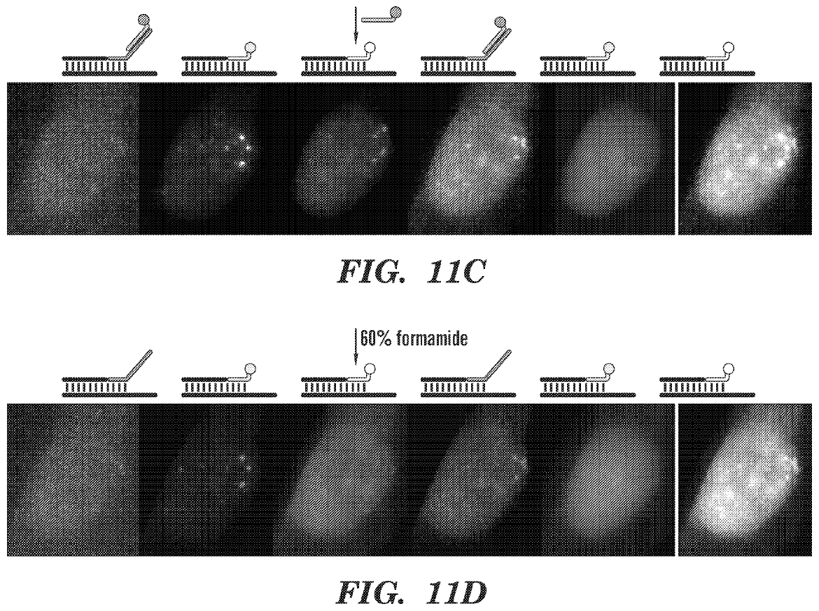

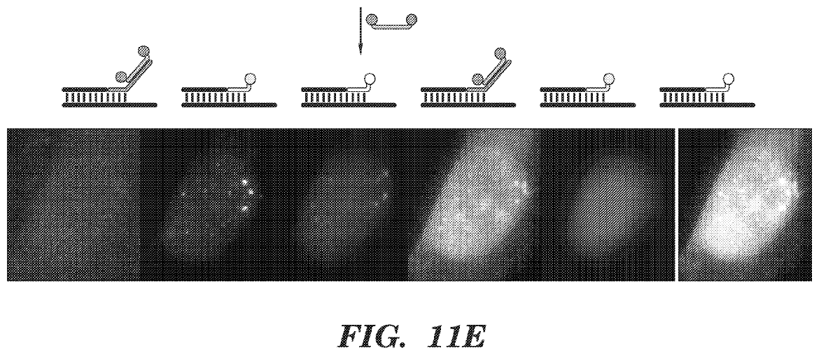

In some embodiments of any of the aspects, the readout molecule comprises: (a) a 3′ barcode-hybridizing region, (b) a 5′ non-barcode-hybridizing region, (c) a modification between the 3′ region and 5′ region, and optionally (d) a detectable label (see e.g., FIG. 10C or FIG. 11A-11E). In some embodiments of any of the aspects, the readout molecule comprises: (a) a 5′ barcode-hybridizing region, (b) a 3′ non-hybridizing region, (c) a modification between the 3′ region and 5′ region, and optionally (d) a detectable label. In some embodiments of any of the aspects, the readout molecule comprises: (a) a barcode-hybridizing region, (b) at least one non-barcode-hybridizing region, (c) a modification between the barcode-hybridizing region and the at least one non-barcode-hybridizing region, and optionally (d) a detectable label.

In some embodiments of any of the aspects, the readout molecule comprises: (a) a 3′ barcode-hybridizing region of nucleotides or analogs thereof comprising a unique sequence distinct from the 3′ region sequence of all other readout molecules in the set; (b) a 5′ region of nucleotides or analogs thereof comprising a sequence identical to the 5′ region sequence of all other readout molecules in the set; (c) a sulfur modification in place of the bridged oxygen of the phosphate backbone between the 5′ and 3′ regions; and optionally (d) an optically detectable label (see e.g., FIG. 10C or FIG. 11A-11E).

In some embodiments of any of the aspects, the readout molecule comprises: (a) a 3′ barcode-hybridizing region, (b) a 5′ non-barcode-hybridizing region, and (c) a modification between the 3′ region and 5′ region (see e.g., FIG. 10C or FIG. 11A-11E). In some embodiments of any of the aspects, the readout molecule comprises: (a) a 5′ barcode-hybridizing region, (b) a 3′ non-hybridizing region, and (c) a modification between the 3′ region and 5′ region. In some embodiments of any of the aspects, the readout molecule comprises: (a) a barcode-hybridizing region, (b) at least one non-barcode-hybridizing region, and (c) a modification between the barcode-hybridizing region and the at least one non-barcode-hybridizing region.

In some embodiments of any of the aspects, the readout molecule comprises: (a) a 3′ barcode-hybridizing region of nucleotides or analogs thereof comprising a unique sequence distinct from the 3′ region sequence of all other readout molecules in the set; (b) 5′ non-barcode-hybridizing region of nucleotides or analogs thereof; and (c) a sulfur modification in place of the bridged oxygen of the phosphate backbone between the 5′ and 3′ regions.

In some embodiments of any of the aspects, the readout molecule comprises: (a) a 3′ barcode-hybridizing region of nucleotides or analogs thereof, the region comprising a unique sequence distinct from the 3′ region sequence of all other readout molecules in the set; (b) a 5′ non-barcode-hybridizing region of nucleotides or analogs thereof, the region comprising a sequence identical to the 5′ region sequence of all other readout molecules in the set; (c) a sulfur modification in place of the bridged oxygen of the phosphate backbone between the 5′ and 3′ regions; (d) an optically detectable label; and (e) a nanoparticle.

In some embodiments of any of the aspects, the readout molecule comprises: (a) a 3′ barcode-hybridizing region of nucleotides or analogs thereof, the region comprising a unique sequence distinct from the 3′ region sequence of all other readout molecules in a set of readout molecules; (b) a 5′ non-barcode-hybridizing region of nucleotides or analogs thereof, the region comprising a sequence identical to the 5′ region sequence of all other readout molecules in the set; (c) a sulfur modification in place of the bridged oxygen of the phosphate backbone between the 5′ and 3′ regions; and (d) a metal nanoparticle.

In some embodiments of any of the aspects, the readout molecule comprises: a 3′ barcode-hybridizing region of nucleotides or analogs thereof, a 5′ non-barcode-hybridizing region of nucleotides or analogs thereof, a sulfur modification in place of the bridged oxygen of the phosphate backbone between the 5′ and 3′ regions; and a metal nanoparticle. In some embodiments of any of the aspects, the readout molecule further comprises an optically detectable label.

As used herein, the term “barcode-hybridizing region” refers to a region of the readout molecule comprising a sequence that is complementary to, and thus hybridizes with, at least a portion of the barcode region of the oligonucleotide tag. In some embodiments of any of the aspects, the barcode-hybridizing region is on the 3′ end of the readout molecule, and thus referred to as the “3′ barcode-hybridizing region.” In some embodiments of any of the aspects, the barcode-hybridizing region is on the 5′ end of the readout molecule, and thus referred to as the “5′ barcode-hybridizing region.” In some embodiments of any of the aspects, the barcode-hybridizing region is between the 5′ and 3′ end of the readout molecule, and referred to as the “barcode-hybridizing region.”

In some embodiments of any of the aspects, the barcode-hybridizing region is 3′ of the non-barcode hybridizing region. In some embodiments of any of the aspects, the barcode-hybridizing region is 5′ of the non-barcode hybridizing region. In some embodiments of any of the aspects, the barcode-hybridizing region is 3′ of the label. In some embodiments of any of the aspects, the barcode-hybridizing region is 5′ of the label.

In some embodiments of any of the aspects, the barcode hybridizing region (e.g., 3′ barcode hybridizing region) is at least 1 nucleotide (or analog thereof) in length. In some embodiments of any of the aspects, the barcode hybridizing region (e.g., 3′ barcode hybridizing region) is 5 nucleotides (and/or analogs thereof) in length. As a non-limiting example, the barcode hybridizing region (e.g., 3′ barcode hybridizing region) is 1 nucleotide, 2 nucleotides, 3 nucleotides, 4 nucleotides, 5 nucleotides, 6 nucleotides, 7 nucleotides, 8 nucleotides, 9 nucleotides, or at least 10 nucleotides in length. In some embodiments of any of the aspects, the length of the barcode-hybridizing region corresponds to the bit size of the barcode region of the oligonucleotide tag, wherein the term “bit” refers to distinct units of the barcode region of the oligonucleotide tag, wherein the bit is at least 1 nucleotide (or analog thereof) long.

In some embodiments of any of the aspects, each barcode-hybridizing region comprises a unique sequence distinct from the barcode-hybridizing region sequence of all other readout molecules in a set of readout molecules. In some embodiments of any of the aspects, each barcode-hybridizing region comprises a unique sequence distinct from the barcode-hybridizing region sequence of at least one (e.g., at least 1, at least 2, at least 3, at least 4, etc.) other readout molecule(s) in a set of readout molecules. In some embodiments of any of the aspects, each type of readout molecule comprises a barcode-hybridizing region sequence that is maximally different from each of the other types of barcode-hybridizing regions in the other types of readout molecules in the set. Differences between the sequences of barcode-hybridizing regions can be measured or quantified by such metrics as Hamming distance. As a non-limiting example, the barcode-hybridizing regions differ from each other by a Hamming distance of at least 2 base-pairs, at least 3 base-pairs, at least 4 base-pairs, at least 5 base-pairs, at least 6 base-pairs, at least 7 base-pairs, at least 8 base-pairs, at least 9 base-pairs, or at least 10 base-pairs.

In some embodiments of any of the aspects, the barcode-hybridizing regions comprises nucleotides and/or nucleotide analogs, as described further herein. Non-limiting examples of barcode-hybridizing region sequences include the following (from 5′ to 3′): GAGCG (SEQ ID NO: 1); GACCA (SEQ ID NO: 2); AGACC (SEQ ID NO: 3); and TGACT (SEQ ID NO: 4); see e.g., FIG. 10C. Additional non-limiting examples of barcode-hybridizing region sequences include the following: GCGAG (SEQ ID NO: 14); ACCAG (SEQ ID NO: 15); CCAGA (SEQ ID NO: 16); and TCAGT (SEQ ID NO: 17). It is anticipated that any given sequence of nucleotide or nucleotide analogs can be a barcode-hybridizing region, inasmuch as each barcode-hybridizing region is distinct and distinguishable from all other barcode-hybridizing regions in a set of readout molecules.

In some embodiments of any of the aspects, the readout molecule comprises at least one other region in addition to the barcode-hybridizing region. In some embodiments of any of the aspects, the readout molecule comprises a 3′ barcode-hybridizing region and a 5′ non-barcode-hybridizing region. In some embodiments of any of the aspects, the readout molecule comprises a 5′ barcode-hybridizing region and a 3′ non-barcode-hybridizing region. In some embodiments of any of the aspects, the readout molecule comprises a barcode-hybridizing region, a 5′ non-barcode-hybridizing region, and a 3′ non-barcode-hybridizing region.

In some embodiments of any of the aspects, the non-barcode hybridizing region (e.g., the 5′ non-barcode-hybridizing region) comprises nucleotides and/or nucleotide analogs as described further herein. In some embodiments of any of the aspects, the non-barcode hybridizing region (e.g., the 5′ non-barcode-hybridizing region) comprises a sequence that is identical to the non-barcode hybridizing region (e.g., 5′ non-barcode-hybridizing region) sequence of other all other readout molecules in the set of readout molecules. In some embodiments of any of the aspects, the non-barcode hybridizing region (e.g., the 5′ non-barcode-hybridizing region) comprises a sequence that is identical to the non-barcode hybridizing region (e.g., 5′ non-barcode-hybridizing region) sequence of at least one (e.g., at least 1, at least 2, at least 5, at least 4, etc.) other readout molecule(s) in the set of readout molecules. In some embodiments of any of the aspects, the non-barcode hybridizing region (e.g., the 5′ non-barcode-hybridizing region) comprises a sequence that is not identical to the non-barcode hybridizing region (e.g., 5′ non-barcode-hybridizing region) sequence of at least one (e.g., at least 1, at least 2, at least 5, at least 4, etc.) other readout molecule(s) other readout molecules in the set.

In some embodiments of any of the aspects, the non-barcode-hybridizing region (e.g., the 5′ non-barcode-hybridizing region and/or 3′ region non-barcode-hybridizing region) comprises only universal nucleotide bases. In some embodiments of any of the aspects, the non-barcode-hybridizing region (e.g., the 5′ non-barcode-hybridizing region and/or 3′ region non-barcode-hybridizing) comprises only deoxyinosine nucleotides.

In some embodiments of any of the aspects, the non-barcode hybridizing region (e.g., the 5′ non-barcode-hybridizing region) is at least 1 nucleotide (or analog thereof) in length. In some embodiments of any of the aspects, the non-barcode hybridizing region (e.g., the 5′ non-barcode-hybridizing region) is 3 nucleotides (and/or analogs thereof) in length. As a non-limiting example, the non-barcode hybridizing region (e.g., the 5′ non-barcode-hybridizing region) is 1 nucleotide, 2 nucleotides, 3 nucleotides, 4 nucleotides, 5 nucleotides, 6 nucleotides, 7 nucleotides, 8 nucleotides, 9 nucleotides, or at least 10 nucleotides in length.

In some embodiments of any of the aspects, the non-barcode-hybridizing region of at least one readout molecule is not linked to a detectable label. In some embodiments of any of the aspects, the non-barcode-hybridizing region of at least one readout molecule specifically hybridizes to an oligonucleotide. In some embodiments of any of the aspects, the oligonucleotide comprises at least one detectable label. In some embodiments of any of the aspects, the oligonucleotide specifically hybridizes to at least one other oligonucleotide (e.g., a branching reaction). In some embodiments of any of the aspects, the oligonucleotide is an amplification primer. In some embodiments of any of the aspects, the oligonucleotide is a sequencing primer. In some embodiments of any of the aspects, the oligonucleotide is an imager strand for super resolution microscopy (e.g., DNA-PAINT).

In some embodiments of any of the aspects, the non-barcode-hybridizing region of at least one readout molecule is at least 5 nucleotides long. In some embodiments of any of the aspects, the non-barcode-hybridizing region of at least one readout molecule is at least 10 nucleotides long. In some embodiments of any of the aspects, the non-barcode-hybridizing region of at least one readout molecule is at least 1, at least 2, at least 3, at least 4, at least 5, at least 6, at least 7, at least 8, at least 9, at least 10, at least 15, at least 20, least 25, at least 30, at least 35, at least 40, at least 45, or at least 50 nucleotides long. In some embodiments of any of the aspects, the non-barcode-hybridizing region comprises a sequence identical to the non-barcode-hybridizing region sequence of all other readout molecules in the set.

In some embodiments of any of the aspects, the readout molecule comprises a modification between the barcode-hybridizing region and the non-barcode-hybridizing region. In some embodiments of any of the aspects, the readout molecule comprises a modification of the phosphate backbone between the barcode-hybridizing region and the non-barcode-hybridizing region, e.g., the backbone chemistry is something other than the naturally occurring phosphate backbone of a nucleic acid. In some embodiments of any of the aspects, the readout molecule comprises a modification between the 3′ barcode-hybridizing region and the 5′ non-barcode-hybridizing region. In some embodiments of any of the aspects, the readout molecule comprises a modification between the 5′ barcode-hybridizing region and the 3′ non-barcode-hybridizing region. In some embodiments, the readout molecule comprises a modification between the 5′ end of the barcode-hybridizing region and the 3′ end of the non-barcode-hybridizing region. In some embodiments, the readout molecule comprises a modification between the 3′ end of the barcode-hybridizing region and the 5′ end of the non-barcode-hybridizing region.

In some embodiments of any of the aspects, the modification comprises a sulfur modification in place of the bridged oxygen of the phosphate backbone of the readout molecule (see e.g., FIG. 10A). By “sulfur modification” is meant the addition of at least one sulfur atom to the phosphate backbone, either as an additional or substitute moiety. In some embodiments of any of the aspects, the modification comprises a phosphorothiolate cleavage site. In some embodiments of any of the aspects, the modification comprises a cleavable modification in the backbone (see e.g., US 2014/0349294; Xu and Kool, Nucleic Acids Res. 1998 Jul. 1, 26(13):3159-64; the contents of each of which are incorporated herein by reference in their entirety. In some embodiments of any of the aspects, the readout molecule comprises any cleavable modification as described further herein.

In some embodiments of any of the aspects, the readout molecule comprises nnnTGACT (SEQ ID NO: 6), nnnAGACC (SEQ ID NO: 7), nnnGACCA (SEQ ID NO: 8), or nnnGAGCG (SEQ ID NO: 9). In some embodiments of any of the aspects, “n” (e.g., in one of SEQ ID NOs: 6-9) comprises a universal nucleotide base (e.g., deoxyinosine). In some embodiments of any of the aspects, the oligo further comprises a sulfur modification in place of the bridged oxygen of the phosphate backbone between nucleotides 3 and 4 (e.g., in one of SEQ ID NOs: 6-9).

In some embodiments of any of the aspects, the readout molecule comprises a detectable label, e.g., an optically detectable molecule i.e., a label that is detectable through light or other electromagnetic wavelengths. In some embodiments of any of the aspects, at least one readout molecule (e.g., of a set of readout molecules) comprises a detectable label. In some embodiments of any of the aspects, at least one readout molecule (e.g., of a set of readout molecules) comprises an optically detectable label. In some embodiments of any of the aspects, the detectable label is a fluorescent label. In some embodiments of any of the aspects, the detectable label is a fluorophore, and detecting is performed with fluorescence microscopy. In some embodiments of any of the aspects, the readout molecule comprises an optically detectable label. In some embodiments of any of the aspects, the detectable label comprises biotin, amines, metals, metal nanoclusters (e.g., gold, silver, or copper), noble metal nanoparticles, anchoring molecules, quantum dots, acrydite, or DNA origami structures. In some embodiments of any of the aspects, the detectable label comprises DNA origami structures (i.e., nanoscale folding of DNA to create non-arbitrary two- and three-dimensional shapes); see e.g., Rothemund, “Folding DNA to create nanoscale shapes and patterns”, Nature 440, 297-302 (2006). In some embodiments of any of the aspects, the detectable labels are detected using electron microscopy, fluorescence microscopy, dark field microscopy, or any combination thereof. In some embodiments of any of the aspects, the readout molecule can comprise any detectable label. Non-limiting examples of detectable labels, fluorophores, and detection techniques are described further herein. In some embodiments of any of the aspects, each readout molecule comprises at least one detectable label. As a non-limiting example, each readout molecule comprises at least 1, at least 2, at least 3, at least 4, or at least 5 detectable labels.

In some embodiments of any of the aspects, a detectable label can be linked to the 5′ end of the readout molecule, a detectable label can be linked to the 3′ end of the readout molecule, or a detectable label can be linked to the 5′ end and the 3′ end of the readout molecule. In some embodiments of any of the aspects, the detectable label linked to the 5′ end of the readout molecule is the same type of detectable label as the detectable label linked to the 3′ end of the readout molecule. In some embodiments of any of the aspects, the detectable label linked to the 5′ end of the readout molecule is a different type of detectable label as the detectable label linked to the 3′ end of the readout molecule.

In some embodiments of any of the aspects, the detectable label is located at the 5′ end of the readout molecule and is linked to the 5′ non-barcode-hybridizing region. In some embodiments of any of the aspects, the detectable label is located at the 3′ end of the readout molecule and is linked to the 3′ non-barcode-hybridizing region. In some embodiments of any of the aspects, the detectable label is cleaved from the readout molecule following detection.

In some embodiments of any of the aspects, at least two readout molecules collectively comprise at least two distinguishable detectable labels. As a non-limiting example, two readout molecules collectively comprise two distinguishable detectable labels, three readout molecules collectively comprise three distinguishable detectable labels, four readout molecules collectively comprise four distinguishable detectable labels, or at least five readout molecules collectively comprise at least five distinguishable detectable labels. In some embodiments of any of the aspects, a pool of readout molecules comprises more readout molecules than distinguishable detectable labels, e.g., the same detectable label can be present on multiple readout molecules.

In some embodiments of any of the aspects, a set of readout molecules described herein comprises at least two labels, wherein the 3′ regions and labels in the set are organized in corresponding pairs, such that any readout molecule comprising a first 3′ region also comprises a corresponding first label, any readout molecule comprising a second 3′ region also comprises a corresponding second label, and each readout molecule does not comprise a label which does not correspond to its 3′ region.

In some embodiments of any of the aspects, a set of readout molecules comprises four distinguishable labels. In some embodiments of any of the aspects, a set of readout molecules comprises at least 2 distinguishable labels, at least 3 distinguishable labels, or at least 4 distinguishable labels. In some embodiments of any of the aspects, a set of readout molecules comprises 5, 6, 7, 8, 9, 10, or more distinguishable labels. In some embodiments of any of the aspects, a set of readout molecules comprises more than four distinguishable labels, which can be accomplished using additional labels (e.g., more than four distinguishable fluorophores), additional barcode bits (e.g., more than four unique barcode bits), and additional barcode-hybridizing regions of the readout molecules (e.g., more than four unique barcode-hybridizing regions, wherein each unique barcode-hybridizing region hybridizes with one of the more than four unique barcode bits).

In some embodiments of any of the aspects, a readout molecule as described herein comprises an optically detectable label and a nanoparticle. In some embodiments of any of the aspects, a readout molecule as described herein comprises a metal particle nanoparticle. The nanoparticle (e.g., a metal nanoparticle) enhances the fluorescence of the detectable label via Plasmon enhanced coupling in a distance dependent manner. In some embodiments of any of the aspects, the nanoparticle (e.g., a metal nanoparticle) is linked to at least one readout molecule as described herein. In some embodiments of any of the aspects, the at least one readout molecule linked to a nanoparticle (e.g., a metal nanoparticle) comprises an optically detectable label (e.g., at the distal end of the readout molecule compared to the linkage to the nanoparticle). In some embodiments of any of the aspects, the nanoparticle (e.g., a metal nanoparticle) is linked to at least two readout molecules, which can be the same or different readout molecules as described herein. As a non-limiting example, the nanoparticle (e.g., a metal nanoparticle) is linked to at least 2, at least 3, at least 4, at least 5, at least 6, at least 7, at least 8, at least 9, at least 10, at least 15, at least 20, at least 30, at least 40, at least 50, at least 60, at least 70, at least 80, at least 90, or at least 100 readout molecules as described herein. Such linkage of the nanoparticle to at least two readout molecules can amplify the signal. Thus, the overall signal amplification can result from multiple readout molecules linked to the nanoparticle (e.g., a metal nanoparticle) and/or Plasmon enhancement of fluorescence (e.g., between the nanoparticle, such as a metal nanoparticle, and the optically detectable label, such as a fluorophore).

In some embodiments of any of the aspects, the optically detectable label comprises a fluorophore (e.g., TexasRed, FITC, or another fluorophore as described further herein). In some embodiments of any of the aspects, the nanoparticle comprises a metal nanoparticle. Non-limiting examples of metal nanoparticles include, but are not limited to, Au, Ag, Ni, Co, Pt, Pd, Cu, Ti, and Al nanoparticles and combinations thereof. In some embodiments of any of the aspects, the nanoparticle comprises a gold nanoparticle. In some embodiments of any of the aspects, the nanoparticle comprises a gold nanorod.

In some embodiments of any of the aspects, the nanoparticle (e.g., a metal nanoparticle) has a diameter of about 1.2 nm. In some embodiments of any of the aspects, the nanoparticle (e.g., a metal nanoparticle) has a diameter of about 3 nm. In some embodiments of any of the aspects, the nanoparticle (e.g., a metal nanoparticle) has a diameter of about 5 nm. In some embodiments of any of the aspects, the nanoparticle (e.g., a metal nanoparticle) has a diameter of about 10 nm. In some embodiments of any of the aspects, the nanoparticle (e.g., a metal nanoparticle) has a diameter of about 30 nm. In some embodiments of any of the aspects, the nanoparticle (e.g., a metal nanoparticle) has a diameter of about 50 nm. In some embodiments of any of the aspects, the nanoparticle (e.g., a metal nanoparticle) has a diameter of at least 1 nm, at least 2 nm, at least 3 nm, at least 4 nm, at least 5 nm, at least 10 nm, at least 15 nm, at least 20 nm, at least 25 nm, at least 30 nm, at least 35 nm, at least 40 nm, at least 45 nm, at least 50 nm, at least 55 nm, at least 60 nm, at least 65 nm, at least 70 nm, at least 75 nm, at least 80 nm, at least 85 nm, at least 90 nm, at least 95 nm, or at least 100 nm.

In some embodiments of any of the aspects, the nanoparticle is at the 3′ end of the readout molecule. In some embodiments of any of the aspects, the nanoparticle is at the 5′ end of the readout molecule. In some embodiments of any of the aspects, the nanoparticle is at the 3′ end and the 5′ of the readout molecule. In some embodiments of any of the aspects, the nanoparticle is at the 3′ end of the readout molecule, and the optically detectable label is at the 5′ end of the readout molecule. In some embodiments of any of the aspects, the nanoparticle is at the 5′ end of the readout molecule, and the optically detectable label is at the 3′ end of the readout molecule.

In some embodiments of any of the aspects, the nanoparticle is at least 20 nucleotides from the detectable label. In some embodiments of any of the aspects, the nanoparticle is at least 30 nucleotides from the detectable label. In some embodiments of any of the aspects, the nanoparticle is at least at least 5 nucleotides, at least 10 nucleotides, at least 15 nucleotides, at least 20 nucleotides, at least 25 nucleotides, at least 30 nucleotides, at least 35 nucleotides, at least 40 nucleotides, at least 45 nucleotides, at least 50 nucleotides, at least 55 nucleotides, at least 60 nucleotides, at least 65 nucleotides, at least 70 nucleotides, at least 75 nucleotides, at least 80 nucleotides, at least 85 nucleotides, at least 90 nucleotides, at least 95 nucleotides, or at least 100 nucleotides from the detectable label.

In one aspect, described herein are sets of readout molecules. In some embodiments of any of the aspects, a set comprises at least 2, at least 3, at least 4, at least 5, at least 6, at least 7, at least 8, at least 9, or at least 10 readout molecules, as described herein. In some embodiments of any of the aspects, a set comprises at least 2, at least 3, at least 4, at least 5, at least 6, at least 7, at least 8, at least 9, or at least 10 distinct and distinguishable types of readout molecules.

In one aspect, described herein is a set of at least two readout molecules, each readout molecule comprising: (a) a 3′ barcode-hybridizing region of nucleotides or analogs thereof, the region comprising a unique sequence distinct from the 3′ region sequence of all other readout molecules in the set; (b) a 5′ non-barcode-hybridizing region of nucleotides or analogs thereof, the region comprising a sequence identical to the 5′ region sequence of all other readout molecules in the set; (c) a sulfur modification in place of the bridged oxygen of the phosphate backbone between the 5′ and 3′ regions; and (d) an optically detectable label.

In one aspect, described herein is a set of at least two readout molecules, each readout molecule comprising: (a) a 3′ barcode-hybridizing region of nucleotides or analogs thereof, the region comprising a unique sequence distinct from the 3′ region sequence of all other readout molecules in the set; (b) a 5′ non-barcode-hybridizing region of nucleotides or analogs thereof, the region comprising a sequence identical to the 5′ region sequence of all other readout molecules in the set; (c) a sulfur modification in place of the bridged oxygen of the phosphate backbone between the 5′ and 3′ regions; and optionally (d) an optically detectable label.

In one aspect, described herein is a set of at least two readout molecules, each readout molecule comprising: (a) a 3′ barcode-hybridizing region of nucleotides or analogs thereof, the region comprising a unique sequence distinct from the 3′ region sequence of all other readout molecules in the set; (b) a 5′ non-barcode-hybridizing region of nucleotides or analogs thereof; and (c) a sulfur modification in place of the bridged oxygen of the phosphate backbone between the 5′ and 3′ regions.

In one aspect, described herein is a set of at least two readout molecules, each readout molecule comprising: (a) a 3′ barcode-hybridizing region of nucleotides or analogs thereof, the region comprising a unique sequence distinct from the 3′ region sequence of all other readout molecules in the set; (b) a 5′ non-barcode-hybridizing region of nucleotides or analogs thereof, the region comprising a sequence identical to the 5′ region sequence of all other readout molecules in the set; (c) a sulfur modification in place of the bridged oxygen of the phosphate backbone between the 5′ and 3′ regions; and (d) an optically detectable label; wherein at least one readout molecule further comprises a nanoparticle.

In some embodiments of any of the aspects, the readout molecules of each set which comprise a first barcode hybridizing region (e.g., a 3′ barcode hybridizing region) only comprise a first distinguishable label. In other words, each barcode-hybridizing region corresponds with a label that is distinguishable from the labels of any of the other barcode-hybridizing regions. In some embodiments of any of the aspects, the readout molecules of each set which comprise any selected barcode hybridizing region (e.g., a 3′ barcode hybridizing region) only comprise a corresponding given distinguishable label.

In some embodiments of any of the aspects, a first distinguishable label only comprises a first barcode hybridizing region (e.g., a 3′ barcode hybridizing region) of a readout molecule. In other words, each label corresponds with one barcode-hybridizing region that is distinguishable from other barcode-hybridizing regions. In some embodiments of any of the aspects, there is a one-to-one relationship between the number of detectable labels and the types of barcode-hybridizing regions (i.e., types of readout molecules). As a non-limiting example, a readout molecule set can comprise 4 distinguishable optically detectable labels and 4 distinguishable barcode hybridizing regions, wherein each type of readout molecule comprises 1 of the 4 distinguishable labels and no other types of readout molecules in the set comprise that label, and wherein each type of readout molecule comprises 1 of the 4 barcode-hybridizing regions and no other types of readout molecules in the set comprise that barcode-hybridizing region.

In some embodiments of any of the aspects, a readout molecule set comprises 2 distinguishable labels and 2 distinguishable barcode hybridizing regions, wherein each label corresponds to one barcode hybridizing region, and each barcode hybridizing region corresponds to one label. In some embodiments of any of the aspects, a readout molecule set comprises 3 distinguishable labels and 3 distinguishable barcode hybridizing regions, wherein each label corresponds to one barcode hybridizing region, and each barcode hybridizing region corresponds to one label. In some embodiments of any of the aspects, a readout molecule set comprises 4 distinguishable labels and 4 distinguishable barcode hybridizing regions, wherein each label corresponds to one barcode hybridizing region, and each barcode hybridizing region corresponds to one label. In some embodiments of any of the aspects, a readout molecule set comprises 5 distinguishable labels and 5 distinguishable barcode hybridizing regions, wherein each label corresponds to one barcode hybridizing region, and each barcode hybridizing region corresponds to one label. In some embodiments of any of the aspects, a readout molecule set comprises 6 distinguishable labels and 6 distinguishable barcode hybridizing regions, wherein each label corresponds to one barcode hybridizing region, and each barcode hybridizing region corresponds to one label. In some embodiments of any of the aspects, a readout molecule set comprises 7 distinguishable labels and 7 distinguishable barcode hybridizing regions, wherein each label corresponds to one barcode hybridizing region, and each barcode hybridizing region corresponds to one label. In some embodiments of any of the aspects, a readout molecule set comprises 8 distinguishable labels and 8 distinguishable barcode hybridizing regions, wherein each label corresponds to one barcode hybridizing region, and each barcode hybridizing region corresponds to one label. In some embodiments of any of the aspects, a readout molecule set comprises n distinguishable labels and n distinguishable barcode hybridizing regions, wherein each label corresponds to one barcode hybridizing region, and each barcode hybridizing region corresponds to one label.

In some embodiments of any of the aspects, a first distinguishable label comprises a limited pool of barcode hybridizing regions (e.g., 3′ barcode hybridizing regions). In other words, each label corresponds with at least one barcode-hybridizing region, each of which is distinguishable from other barcode-hybridizing regions. As a non-limiting example, each distinguishable label corresponds to 2, 3, 4, 5, 6, 7, 8, 9, or at least 10 distinct barcode hybridizing regions, and no other distinguishable label corresponds to these barcode hybridizing regions. In some embodiments, each distinguishable label corresponds to less than 256 distinct barcode hybridizing regions, e.g., less than 200, less than 100, less than 50, less than 25, less than 10, or less than 5 distinct barcode hybridizing regions. This limited number of barcode hybridizing regions (and thus barcode regions of the oligonucleotide tag) decreases the number of readout molecules needed in each set and increases the signal output of detection methods described herein.

In some embodiments of any of the aspects, in a set of readout molecules each type of readout molecule comprises a distinct nucleotide or nucleotide analog at the 3′ end of the 3′ hybridizing region. As a non-limiting example, in a set of 4 types of readout molecules; 1 readout molecule comprises a 3′ barcode-hybridizing region with an adenine (A) at the first nucleotide (nt) position (i.e., 3′-most nucleotide) of the 3′ barcode-hybridizing region; 1 readout molecule comprises a 3′ barcode-hybridizing region with an thymine (T) at the first nt position of the 3′ barcode-hybridizing region; 1 readout molecule comprises a 3′ barcode-hybridizing region with an cytosine (C) at the first nt position of the 3′ barcode-hybridizing region; and 1 readout molecule comprises a 3′ barcode-hybridizing region with an guanine (G) at the first nt position of the 3′ barcode-hybridizing region.

In some embodiments of any of the aspects, in set of readout molecules that comprises nucleotides and nucleotide analogs, the first nt position of the 3′ hybridizing region can comprise an A, T, C, G, uracil (U), 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine, 7-daazaadenine, 3-deazaguanine, 3-deazaadenine, or any other nucleotide analog described herein. In some embodiments of any of the aspects, the least two readout molecules of the set are DNA and/or RNA. In some embodiments of any of the aspects, the least two readout molecules of the set comprise DNA and/or RNA. In some embodiments of any of the aspects, the least two readout molecules of the set molecules consist of or consist essentially of DNA and/or RNA. In some embodiments of any of the aspects, the least two readout molecules of the set comprise a polypeptide.

In some embodiments of any of the aspects, the set of readout molecule comprises at least one of SEQ ID NOs: 6-9. In some embodiments of any of the aspects, the set of readout molecule comprises at least two of SEQ ID NOs: 6-9. In some embodiments of any of the aspects, the set of readout molecule comprises at least three of SEQ ID NOs: 6-9. In some embodiments of any of the aspects, the set of readout molecule comprises SEQ ID NOs: 6-9.

In one aspect described herein is use of a readout molecule or set thereof, as described herein, for: detection of at least one target molecule; signal amplification; branch reactions; hybridization chain reaction (HCR); signal amplification by exchange reaction (SABER); rolling circle amplification (RCA); in situ sequencing; matrix attachment; or super resolution microscopy. In some embodiments of any of the aspects, the readout molecule or set thereof can be attached to a matrix, including, but not limited to, a nuclear matrix, a cellular matrix, or a hydrogel.

In one aspect described herein is a method of detecting at least one target molecule in a sample. In some embodiments of any of the aspects, the method comprises contacting the sample with at least one oligonucleotide tag. In some embodiments of any of the aspects, each oligonucleotide tag comprises a recognition domain that binds specifically to a target molecule to be detected and a street comprising a barcode region.

As used herein, the term “oligonucleotide tag” is an oligonucleotide that comprises a recognition domain and/or at least one street. In some embodiments of any of the aspects, the oligonucleotide tag comprises or is comprised by oligonucleotides including but not limited to Oligopaints, multiplexed error-robust fluorescence in situ hybridization (MERFISH) oligos, seqFISH oligos, RNA sequential probing of targets (SPOTs) oligos, high-coverage microscopy-based technology (Hi-M) oligos, or optical reconstruction of chromatin architecture (ORCA) oligos or any to oligonucleotide used for FISH methods and/or any oligonucleotides that has a sequence complementary (e.g. recognition domain) to a target molecule, e.g., an oligonucleotide sequence, a portion of a DNA sequence, or a particular chromosome or sub-chromosomal region of a particular chromosome. For further details, see e.g., Cardozo et al., Mol Cell. 2019 Apr. 4; 74(1):212-222; Mateo et al., Nature. 2019 April; 568(7750):49-54; Wang et al., Scientific Reports volume 8, Article number: 4847 (2018); Shah et al., Neuron, Volume 92, Issue 2, 19 Oct. 2016, Pages 342-357; Eng et al., Nat Methods. 2017 Dec.; 14(12):1153-1155; the contents of each of which is incorporated herein by reference in its entirety.

As used herein, the term “street” refers to a portion of the oligonucleotide tag (e.g., an Oligopaint) that does not have identity with a target sequence or does not hybridize to a target sequence. In some embodiments of any of the aspects, the street comprises a barcode region. As used herein, “barcode region” refers to a region of a cassette comprising at least 1 nucleotide. As a non-limiting example, the barcode region comprises 1 nucleotide, 2 nucleotides, 3 nucleotides, 4 nucleotides, 5 nucleotides, 6 nucleotides, 7 nucleotides, 8 nucleotides, 9 nucleotides, or 10 nucleotides. In some embodiments of any of the aspects, at least one nucleotide of the barcode region comprises a modified nucleobase base, as described further herein.

In some embodiments of any of the aspects, the barcode region is unique to each oligonucleotide tag. As described herein, each barcode region of the oligonucleotide tag comprises at least one bit or unit, and each bit corresponds to at least one barcode-hybridizing region that is complementary to at least a portion of the bit. As a non-limiting example, each bit of the oligonucleotide tag barcode region corresponds to 1 barcode-hybridizing region of a readout molecule, 2 barcode-hybridizing regions of 2 readout molecules, 3 barcode-hybridizing regions of 3 readout molecules, 4 barcode-hybridizing regions of 4 readout molecules, or at least 5 barcode-hybridizing regions of at least 5 readout molecules, wherein each barcode-hybridizing region comprises a unique sequence that is complementary and hybridizes to at least a portion of the bit of the barcode region of the oligonucleotide tag.

In some embodiments of any of the aspects, a bit is 1 nucleotide, 2 nucleotides, 3 nucleotides, 4 nucleotides, 5 nucleotides, 6 nucleotides, 7 nucleotides, 8 nucleotides, 9 nucleotides, or at least 10 nucleotides in length. In some embodiments of any of the aspects, the barcode region of the oligonucleotide tag comprises at least 1 bit. As a non-limiting example, the barcode region of the oligonucleotide tag comprises 1 bit, 2 bits, 3 bits, 4 bits, 5 bits, 6 bits, 7 bits, 8 bits, 9 bits, or at least 10 bits.

In some embodiments of any of the aspects, the sequence of the barcode region differs from the barcode regions of the other oligonucleotide tags, in that the selection and/or order of barcode bits is unique to each oligonucleotide tag. In some embodiments of any of the aspects, each and every oligonucleotide tag has a different barcode region, e.g., a barcode region with a different sequence of barcode bits and/or nucleotides. In some embodiments of any of the aspects, the street of an oligonucleotide tag comprises at least 2 barcode regions, and each and every barcode region in a street is different than the other barcode regions in the street, e.g., a barcode region with a different sequence of barcode bits and/or nucleotides. In some embodiments of any of the aspects, the sequence of the barcode region is the same and shared with at least one barcode region of the other oligonucleotide tags.

In some embodiments of any of the aspects, each oligonucleotide tag's street is unique from the streets of the other oligonucleotide tags due to its barcode region or ordered set of barcode bits. In some embodiments of any of the aspects, each oligonucleotide tag's street is unique from the streets of the other oligonucleotide tags at least in that the spatial order of the barcode bits within the street differs. As a non-limiting example, a barcode comprising three barcode bits (e.g., barcode bits “A”, “B”, and “C”) in the spatial order 5′-A-B-C-3′ has a unique spatial order of barcode bits that differs compared to any other streets comprising a spatial order of barcode bits selected from 5′-A-C-B-3′, 5′-B-A-C 3′, 5′-B-C-A 3′, 5′-C-A-B 3′, or 5′-C-B-A 3′, and each of the aforementioned streets are unique and differ from each other in their spatial order of their barcode bits.

In some embodiments of any of the aspects, each oligonucleotide tag's (e.g., an Oligopaint) street is unique from the streets of the other oligonucleotide tags at least in that the spatial order of the barcode bits within the street differs, and each oligonucleotide tag's street is unique from the streets of the other oligonucleotide tags at least in that the barcode region or barcode regions within the street differs.

In some embodiments of any of the aspects, the street of the oligonucleotide tag further comprises a primer binding region for annealing a sequencing primer. In some embodiments of any of the aspects, the sequencing primer is DNA or RNA, and comprises nucleotides and/or nucleotide analogs. In some embodiments of any of the aspects, the sequencing primer is at least 5 nucleotides, at least 10 nucleotides, at least 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, or at least 25 nucleotides long. In some embodiments of any of the aspects, the sequencing primer comprises a 5′ phosphate. In some embodiments of any of the aspects, the primer binding region is at least 5 nucleotides, at least 10 nucleotides, at least 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, or at least 25 nucleotides long.

In some embodiments of any of the aspects, the primer binding region is located 5′ to the barcode region of the oligonucleotide tag. In some embodiments of any of the aspects, the primer binding region is located immediately 5′ to the barcode region of the oligonucleotide tag.

In some embodiments of any of the aspects, the oligonucleotide tag (e.g., an Oligopaint) comprises a recognition domain. As used herein, a “recognition domain” is a domain of the oligonucleotide tag (e.g., an Oligopaint) that binds specifically to a target molecule and/or sequence to be detected. As a non-limiting example, the recognition domain can be a nucleic acid sequence that is complementary to a target molecule and/or sequence, e.g., a region of a chromosome. Accordingly, the sequence of the recognition domain will vary depending on the identity of the desired target. It is well within the skill of the art to design a recognition domain that will specifically hybridize to any given target under specific conditions, e.g., using software widely and freely available for this purpose (e.g., Primer3 or PrimerBank, which are both available on the world wide web). In some embodiments of any of the aspects, the recognition domain can have at least 90%, at least 91%, at least 92%, at least 93%, at least 94%, at least 95%, at least 96%, at least 97%, at least 98%, at least 99%, or 100% identity with a portion of the target molecule and/or with the target sequence. In some embodiments of any of the aspects, the recognition domain comprises a domain of “genomic homology” or a domain that specifically binds to a region of the genome. In some embodiments of any of the aspects, multiple recognition domains found on the same or different oligonucleotide tags (e.g., an Oligopaint) can specifically bind to a single target molecule and/or target sequence. As a non-limiting example, at least 2 recognition domains, at least 3 recognition domains, at least 4 recognition domains, at least 5 recognition domains, at least 10 recognition domains, at least 20 recognition domains, at least 30 recognition domains, at least 40 recognition domains, or at least 50 recognition domains can specifically bind to a target molecule and/or target sequence.

In some embodiments of any of the aspects, when multiple oligonucleotide tags are present, the multiple recognition domain sequences can each be unique relative to the other recognition domains present. In some embodiments of any of the aspects, when multiple oligonucleotide tags are present, the multiple recognition domain sequences do not overlap.

In some embodiments of any of the aspects, the recognition domain comprises or is comprised by oligonucleotides including but not limited to Oligopaints, multiplexed error-robust fluorescence in situ hybridization (MERFISH) oligos, seqFISH oligos, RNA sequential probing of targets (SPOTs) oligos, high-coverage microscopy-based technology (Hi-M) oligos, or optical reconstruction of chromatin architecture (ORCA) oligos or any to oligonucleotide used for FISH methods and/or any oligonucleotides that has a sequence complementary (e.g. recognition domain) to a target molecule, e.g., an oligonucleotide sequence, a portion of a DNA sequence, or a particular chromosome or sub-chromosomal region of a particular chromosome. For further details, see e.g., Cardozo et al., Mol Cell. 2019 Apr. 4; 74(1):212-222; Mateo et al., Nature. 2019 April; 568(7750):49-54; Wang et al., Scientific Reports volume 8, Article number: 4847 (2018); Shah et al., Neuron, Volume 92, Issue 2, 19 Oct. 2016, Pages 342-357; Eng et al., Nat Methods. 2017 Dec.; 14(12):1153-1155; each of which is incorporated herein by reference in its entirety.

In some embodiments of any of the aspects, the recognition domain comprises a non-nucleic acid, e.g., any nucleic-acid binding composition such as a DNA-binding polypeptide. As a non-limiting example, the recognition domain comprises a sequence-specific single-stranded DNA binding protein or factor, a sequence-specific double-stranded DNA binding protein or factor, a DNA-RNA binding protein or factor, or an RNA binding protein or factor. Non-limiting examples of such a nucleic-acid-binding composition include but are not limited to a transcription factor, a restriction enzyme, a transcription activator-like effector nuclease (TALENs), a CRISPR-Cas-type factor, and the like. In some embodiments of any of the aspects, the nucleic-acid-binding composition lacks nuclease activity.

In some embodiments of any of the aspects, the target molecule comprises a non-nucleic acid, e.g., a polypeptide. Accordingly, the recognition domain comprises any composition that specifically binds a target polypeptide. Non-limiting examples of such a polypeptide-binding recognition domain include but are not limited to an antibody (e.g., a nanobody), an aptamer, a small molecule, a ligand, a known binding partner of a specific polypeptide, and the like.

In some embodiments of any of the aspects, the oligonucleotide tag does not specifically recognize a target molecule, in that the oligonucleotide tag is not linked to a recognition domain but is linked to an entity for detecting the oligonucleotide-tagged entity of interest. The oligonucleotide tag can be a nucleic acid comprising at one anchor region and at least barcode region, but e.g., lacking a recognition domain. As non-limiting examples, such oligonucleotide-tagged entities can include small molecules (e.g., for the purpose of drug screens), polypeptides, cells, or non-biological materials (e.g., metals, chemicals, etc.). The methods of detecting such oligonucleotide-tagged entities can be identical to those used for detecting oligonucleotide tags (e.g., Oligopaint) as described herein (e.g., contacting with pools of readout molecules that hybridize to the specific cassette types in the oligonucleotide tags). In some embodiments of any of the aspects, multiple types of target molecules and/or types of tagged entities can be detected at once, e.g., using at least one oligonucleotide tag (e.g., Oligopaint) that recognizes DNA, at least one oligonucleotide tag that recognizes polypeptides, and/or at least one-oligonucleotide-tagged entity.

In some embodiments of any of the aspects, the oligonucleotide tag (e.g., Oligopaint) comprises at least one street. As used herein, the term “street” refers to a portion of the oligonucleotide tag (e.g., Oligopaint) that does not have identity with a target sequence or does not hybridize to a target sequence. Streets comprises regions for detection and/or regions for amplification. As a non-limiting example, the oligonucleotide tag (e.g., Oligopaint) comprises two streets. In some embodiments of any of the aspects, the street can be one or more of a “Mainstreet” and/or a “Backstreet”. As a non-limiting example, the Mainstreet is 5′ to the recognition domain, the Mainstreet is 5′ to the Backstreet, and/or the Mainstreet is 5′ to the recognition domain and the Backstreet. As a non-limiting example, the Backstreet is 3′ to the recognition domain, the Backstreet is 3′ to the Mainstreet, and/or the Backstreet is 3′ to the recognition domain and the Mainstreet. As a non-limiting example, the Mainstreet is 3′ to the recognition domain, the Mainstreet is 3′ to the Backstreet, and/or the Mainstreet is 3′ to the recognition domain and the Backstreet. As a non-limiting example, the Backstreet is 5′ to the recognition domain, the Backstreet is 5′ to the Mainstreet, and/or the Backstreet is 5′ to the recognition domain and the Mainstreet.

In some embodiments of any of the aspects, the street (e.g., Mainstreet and/or Backstreet) comprises at least one barcode region and/or at least one universal primer binding region. As used herein, “universal primer binding region” refers to a region that binds a universal primer (e.g., a universal forward primer, a universal reverse primer). As used herein, “universal primer” refers to a primer that is used for multiple individual oligonucleotide tags (e.g., Oligopaint) or a set of oligonucleotide tags. Universal primers can be used for the purpose of amplifying, for example with PCR, the oligonucleotide tag (e.g., Oligopaint), e.g., for production of the oligonucleotide tag or set of oligonucleotide tags. In some embodiments of any of the aspects, the universal primer binding region of each oligonucleotide tag (e.g., Oligopaint) is identical to the universal primer binding region of the remaining oligonucleotide tags, e.g., any other oligonucleotide tag the sample is contacted with.

In some embodiments of any of the aspects, the street comprises at least one universal primer binding region and/or at least one barcode region. As a non-limiting example, the universal primer binding region is 5′ of at least barcode region. As a non-limiting example, the universal forward primer binding region, which specifically binds to a universal forward primer, is at the 5′ end of the oligonucleotide tag (e.g., Oligopaint). As a non-limiting example, the universal primer binding region is 3′ of at least one barcode region. As a non-limiting example, the universal reverse primer binding region, which specifically binds to a universal reverse primer, is at the 3′ end of the oligonucleotide tag (e.g., Oligopaint). In some embodiments of any of the aspects, universal primer binding regions flank (both 5′ and 3′) any barcode region present in the oligonucleotide tag (e.g., Oligopaint). In some embodiments of any of the aspects, the universal reverse primer binding region comprises a recognition site for a nicking endonuclease (NE), e.g., to cause the oligonucleotide tag (e.g., Oligopaint) to become single-stranded when exposed to an NE. In some embodiments of any of the aspects, the oligonucleotide tag (e.g., Oligopaint) is not necessarily amplified (e.g., through PCR and/or universal priming regions). In some embodiments of any of the aspects, the oligonucleotide tag (e.g., Oligopaint) described can be synthesized, de novo, and used “straight from the tube”.

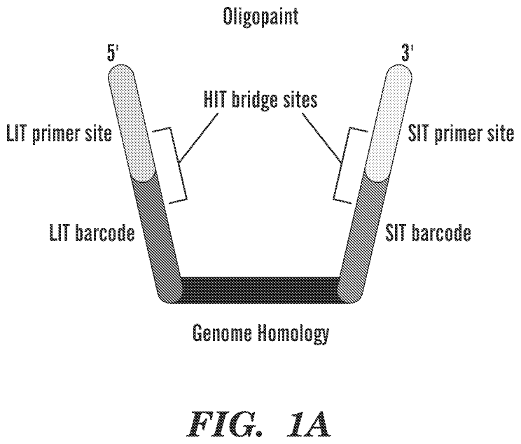

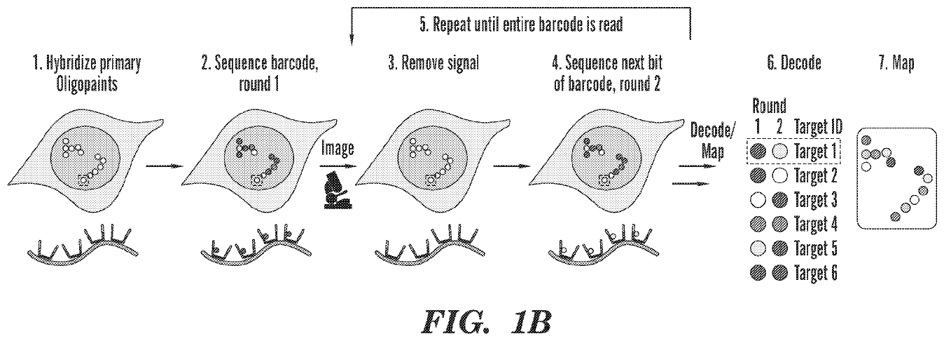

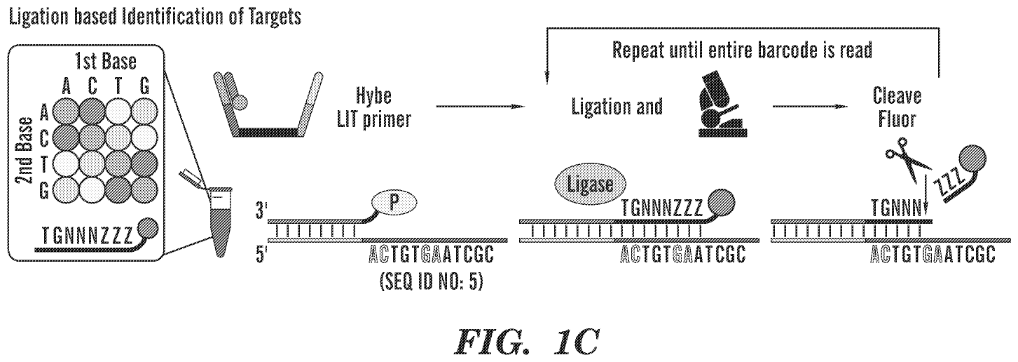

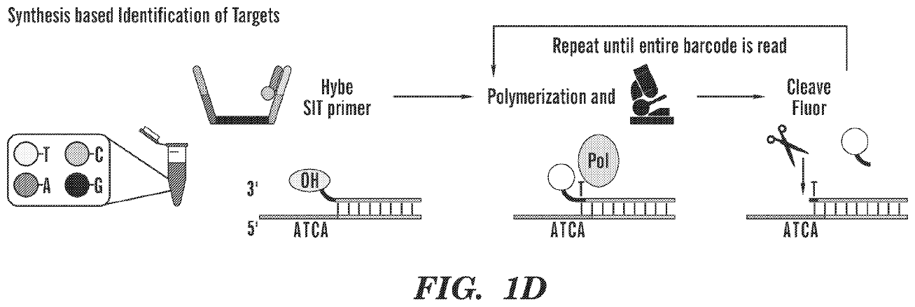

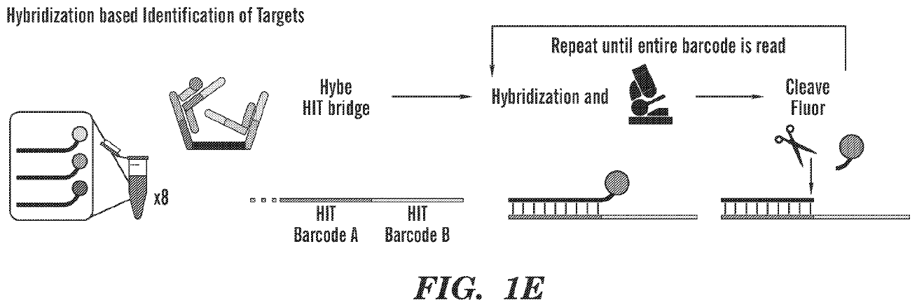

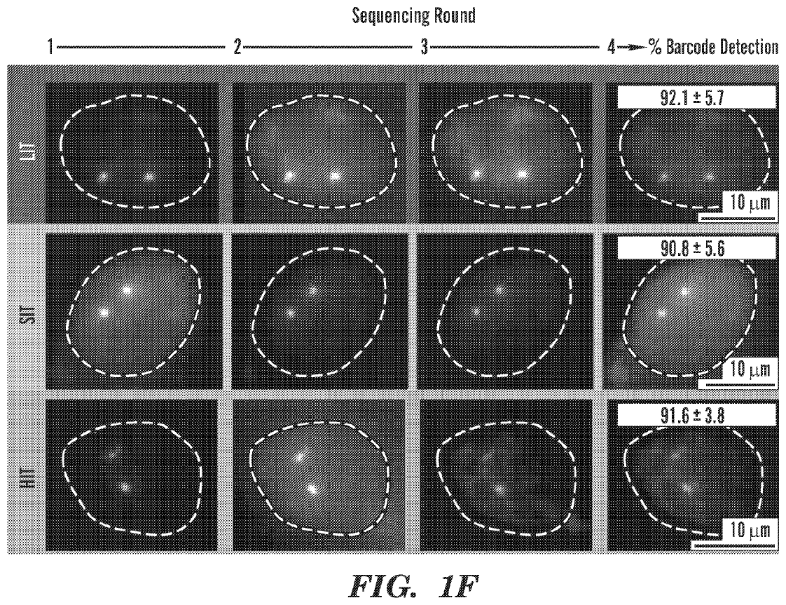

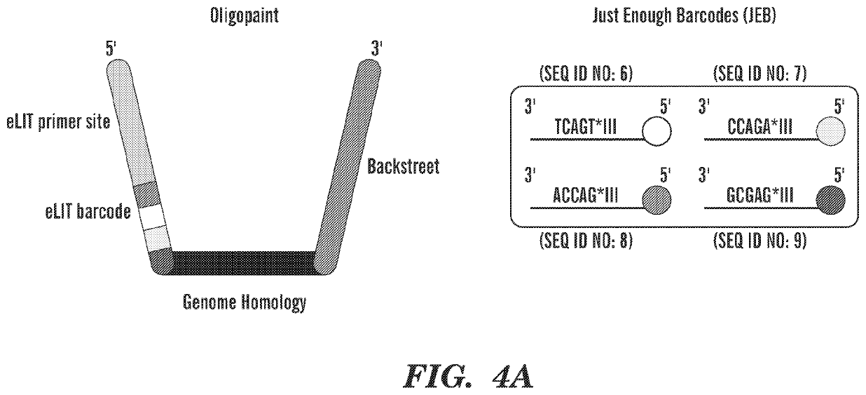

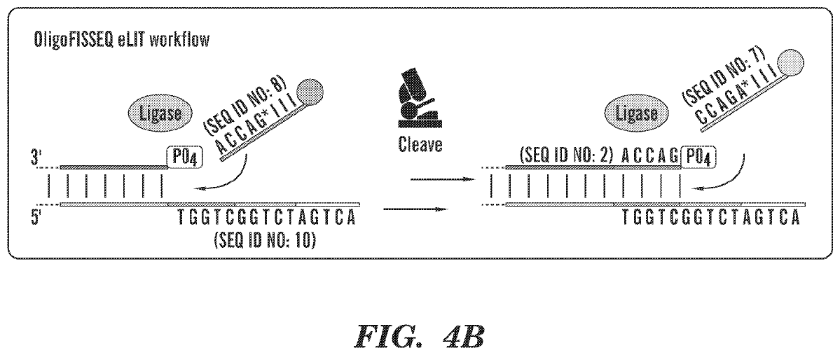

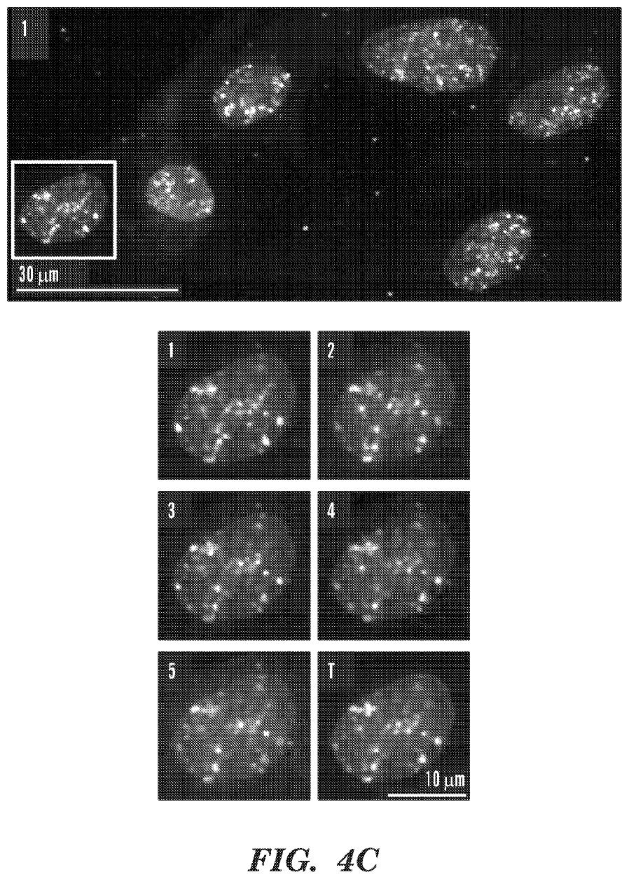

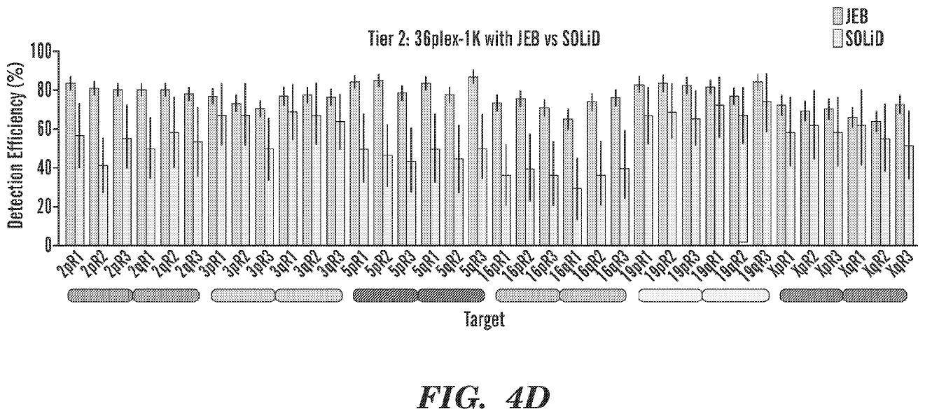

In some embodiments of any of the aspects, the oligonucleotide tag comprises a Ligation-based Identification of Targets (LIT) primer binding site and a LIT barcode region; in other words, the oligonucleotide tag comprises a primer binding site and barcode region that are detected using a sequencing by ligation (SBL) method as described herein (see e.g., FIG. 1A, FIG. 1C, FIG. 4A, FIG. 10D). In some embodiments of any of the aspects, oligonucleotide tag Mainstreet comprises a LIT primer binding site and a LIT barcode (see e.g., FIG. 1A, FIG. 4A, FIG. 10D). In some embodiments of any of the aspects, oligonucleotide tag Backstreet comprises a LIT primer binding site and a LIT barcode region (see e.g., FIG. 1A, FIG. 4A, FIG. 10D). In some embodiments of any of the aspects, oligonucleotide tag Mainstreet and Backstreet each comprise a LIT primer binding site and a LIT barcode region (see e.g., FIG. 1A, FIG. 4A, FIG. 10D).

In some embodiments of any of the aspects, the oligonucleotide tag comprises an exact Ligation-based Identification of Targets (eLIT) primer binding site and an eLIT barcode region; in other words the oligonucleotide tag comprises a primer binding site and barcode region that are detected using a sequencing by ligation (SBL) method comprising a set of readout molecules as described herein (see e.g., FIG. 1A, FIG. 1C, FIG. 4A, FIG. 10D). In some embodiments of any of the aspects, oligonucleotide tag Mainstreet comprises an eLIT primer binding site and an eLIT barcode region (see e.g., FIG. 1A, FIG. 4A, FIG. 10D). In some embodiments of any of the aspects, oligonucleotide tag Backstreet comprises an eLIT primer binding site and an eLIT barcode region (see e.g., FIG. 1A, FIG. 4A, FIG. 10D). In some embodiments of any of the aspects, oligonucleotide tag Mainstreet and Backstreet each comprise an eLIT primer binding site and an eLIT barcode region (see e.g., FIG. 1A, FIG. 4A, FIG. 10D).

In some embodiments of any of the aspects, the oligonucleotide tag further comprises a Synthesis-based Identification of Targets (SIT) primer binding site and a SIT barcode region; in other words, the oligonucleotide tag further comprises a primer binding site and barcode region that are detected using a sequencing by synthesis (SBS) method as described herein (see e.g., FIG. 1A, FIG. 1D). In some embodiments of any of the aspects, the oligonucleotide tag Mainstreet comprises an eLIT primer binding site and an eLIT barcode region, and the oligonucleotide tag Backstreet comprises a SIT primer binding site and a SIT barcode region (see e.g., FIG. 1A). In some embodiments of any of the aspects, the oligonucleotide tag Backstreet comprises an eLIT primer binding site and an eLIT barcode region, and the oligonucleotide tag Mainstreet comprises a SIT primer binding site and a SIT barcode region (see e.g., FIG. 1A). In some embodiments of any of the aspects, the oligonucleotide tag Mainstreet comprises an eLIT primer site, an eLIT barcode region, a SIT primer binding site and a SIT barcode region. In some embodiments of any of the aspects, the oligonucleotide tag Backstreet comprises an eLIT primer site, an eLIT barcode region, a SIT primer binding site and a SIT barcode region.

In some embodiments of any of the aspects, the oligonucleotide tag further comprises a Hybridization-based Identification of Targets (HIT) oligonucleotide binding site and a HIT barcode region; in other words, the oligonucleotide tag further comprises an oligonucleotide binding site and barcode region that are detected using a sequencing by hybridization (SBH) method as described herein (see e.g., FIG. 1A, FIG. 1E). In some embodiments of any of the aspects, a method of detecting the oligonucleotide tag comprising a HIT oligonucleotide binding site and HIT barcode is described further in U.S. Provisional Patent Application No. 62/880,216, filed Jul. 30, 2019, the content of which is incorporated herein by reference in its entirety.

In some embodiments of any of the aspects, the oligonucleotide tag further comprises a HIT oligonucleotide binding site, and a secondary oligonucleotide comprises a HIT barcode region (see e.g., FIG. 1A, FIG. 1E). In some embodiments of any of the aspects, a method of detecting the oligonucleotide tag comprising a HIT oligonucleotide binding site comprises contacting the oligonucleotide tag with a secondary oligonucleotide (also referred to herein as a bridge oligonucleotide or “bridge”), wherein the secondary oligonucleotide comprises at least one barcode region and at least one region that is complementary to and hybridizes to the oligonucleotide binding site of the oligonucleotide tag. In some embodiments of any of the aspects, the method further comprises contacting the secondary oligonucleotide with at least two readout molecules, wherein each readout molecule comprises: an oligonucleotide that hybridizes specifically with a secondary oligonucleotide and a detectable label.

In some embodiments of any of the aspects, the oligonucleotide tag Mainstreet comprises an eLIT primer binding site and an eLIT barcode region, and the oligonucleotide tag Backstreet comprises a HIT oligonucleotide binding site and a HIT barcode region (see e.g., FIG. 1A). In some embodiments of any of the aspects, the oligonucleotide tag Backstreet comprises an eLIT primer binding site and an eLIT barcode region, and the oligonucleotide tag Mainstreet comprises a HIT oligonucleotide binding site and a HIT barcode region (see e.g., FIG. 1A). In some embodiments of any of the aspects, the oligonucleotide tag Mainstreet comprises an eLIT primer binding site, an eLIT barcode region, a HIT oligonucleotide binding site and a HIT barcode region. In some embodiments of any of the aspects, the oligonucleotide tag Backstreet comprises an eLIT primer binding site, an eLIT barcode region, a HIT oligonucleotide binding site and a HIT barcode region.

In some embodiments of any the aspects, the oligonucleotide tag (e.g., Mainstreet and/or Backstreet) comprises any combination of eLIT (or LIT) primer binding site, eLIT (or LIT) barcode region, SIT primer binding site, SIT barcode region, HIT oligonucleotide binding site, and HIT barcode region, as shown in Table 1 below, where “X” indicates that the oligonucleotide tag comprises the site or region.

TABLE 1
Exemplary Combinations of primer binding site(s) and barcode region(s)

| eLIT (or | eLIT (or | | | HIT | |
|---|---|---|---|---|---|
| LIT) primer | LIT) barcode | SIT primer | SIT barcode | oligonucleotide | HIT barcode |
| binding site | region | binding site | region | binding site | region |
| X | | | | | |
| | X | | | | |
| X | X | | | | |
| | | X | | | |
| X | | X | | | |
| | X | X | | | |
| X | X | X | | | |
| | | | X | | |
| X | | | X | | |
| | X | | X | | |
| X | X | | X | | |
| | | X | X | | |
| X | | X | X | | |
| | X | X | X | | |
| X | X | X | X | | |
| | | | | X | |
| X | | | | X | |
| | X | | | X | |
| X | X | | | X | |
| | | X | | X | |
| X | | X | | X | |
| | X | X | | X | |
| X | X | X | | X | |
| | | | X | X | |
| X | | | X | X | |
| | X | | X | X | |
| X | X | | X | X | |
| | | X | X | X | |
| X | | X | X | X | |
| | X | X | X | X | |
| X | X | X | X | X | |
| | | | | | X |
| X | | | | | X |
| | X | | | | X |
| X | X | | | | X |
| | | X | | | X |
| X | | X | | | X |
| | X | X | | | X |
| X | X | X | | | X |
| | | | X | | X |
| X | | | X | | X |
| | X | | X | | X |
| X | X | | X | | X |
| | | X | X | | X |
| X | | X | X | | X |
| | X | X | X | | X |
| X | X | X | X | | X |
| | | | | X | X |
| X | | | | X | X |
| | X | | | X | X |
| X | X | | | X | X |
| | | X | | X | X |
| X | | X | | X | X |
| | X | X | | X | X |
| X | X | X | | X | X |
| | | | X | X | X |
| X | | | X | X | X |
| | X | | X | X | X |
| X | X | | X | X | X |
| | | X | X | X | X |
| X | | X | X | X | X |
| | X | X | X | X | X |
| X | X | X | X | X | X |

Described herein are methods of detecting target molecules in a sample. Accordingly, in one aspect described herein is a method of detecting at least one target molecule in a sample, the method comprising: (a) contacting the sample with at least one oligonucleotide tag, each oligonucleotide tag comprising: (i) a recognition domain that binds specifically to a target molecule to be detected, and (ii) a street comprising a barcode region; (b) contacting the sample with a set of readout molecules as described herein; and (c) detecting the relative order of the optically detectable labels hybridized to the at least one oligonucleotide tag, wherein the at least one oligonucleotide tag is hybridized to the at least one target molecule, whereby the relative order of the optically detectable labels permits identification of which oligonucleotide tag is hybridized to the target molecule at that location. In some embodiments of any of the aspects, the specific hybridization of a readout molecule to a street is determined by the identity of the barcode region and barcode-hybridizing region.

In some embodiments of any of the aspects, the sample is contacted with at least one type of oligonucleotide tag (i.e., comprising the same recognition domain). As a non-limiting example, the sample is contacted with at least 1, at least 2, at least 3, at least 4, or at least 5 types of oligonucleotide tags. In some embodiments of any of the aspects, the sample is contacted with a large set of oligonucleotide tags, in order to “paint” chromosomes. Accordingly, in some embodiments of any of the aspects, the sample is contacted with at least 500, at least 750, at least 1,000, at least 2,000, at least 3,000, at least 4,000, at least 5,000, at least 6,000, at least 7,000, at least 8,000, at least 9,000, at least 10,000, at least 20,000, at least 30,000, at least 40,000, at least 50,000, at least 60,000, at least 70,000, at least 80,000, at least 90,000, or at least 100,000 types of oligonucleotide tags. In some embodiments of any of the aspects, oligonucleotide tags can be used to detect at least one target molecule in a multiplexed manner, i.e., at least two different oligonucleotide tags used concurrently in the same sample.

In some embodiments of any of the aspects, the sample is contacted with at least one set of readout molecules. As a non-limiting example, the sample is contacted with 1 readout molecule set, 2 readout molecule sets, 3 readout molecule sets, 4 readout molecule sets, or at least 5 readout molecule sets.

In some embodiments of any of the aspects, the detecting step comprises detecting the relative spatial order of the readout molecules hybridized to the at least one oligonucleotide tag. The different labels (e.g., colors) of the readout molecules correlate with one or more barcode regions, and the spatial order of the different readout molecules provides information about the order of the barcode regions on a single oligonucleotide tag, allowing a large number of different oligonucleotide tags to be distinguished by the barcoded signals provided by groups of readout molecules. Accordingly, in some embodiments of any of the aspects, the relative spatial order of the detectable labels permits identification of which oligonucleotide tag is hybridized to the target molecule at that location.

In some embodiments of any of the aspects, the barcode region is unique to each oligonucleotide tag, and each barcode region comprises at least 1 barcode bit, as described further herein. In some embodiments of any of the aspects, the total number of unique barcode bits is less than the total number of unique barcode bits possible. As used herein, the phrase “total number of unique barcode bits possible” refers to the number of potential nucleotides and/or nucleotide analogs that can be used to the power of the number of nucleotide and/or nucleotide analog positions in a bit (i.e., the length of the barcode bit). As a non-limiting example, in a barcode bit that is comprised of 4 different nucleotides and is 5 nucleotides long, the total number of unique barcode bits possible is 4\hat{ }5 or 4*4*4*4*4 or 1024.

In some embodiments of any of the aspects, the total number of unique barcode bits is less than 10% of the total number of unique barcode bits bit possible. As a non-limiting example, in a barcode bit that is comprised of 4 different nucleotides and is 5 nucleotides long, the total number of unique barcode bits is less than 100 (˜1024*0.1). In some embodiments of any of the aspects, the total number of unique barcode bits is less than 1% of the total number of unique barcode bits bit possible. As a non-limiting example, in a barcode bit that is comprised of 4 different nucleotides and is 5 nucleotides long, the total number of unique barcode bits is less than 10 (1024*0.01).

In some embodiments of any of the aspects, the total number of unique barcode bits is at least 2 unique barcode bits. As a non-limiting example, the total number of unique barcodes bits is at least 2, at least 3, at least 4, at least 5, at least 6, at least 7, at least 8, at least 9, or at least 10 unique barcode bits. In some embodiments of any of the aspects, the total number of unique barcode bits is no more than 10 unique barcode bits. As a non-limiting example, the total number of unique barcodes bits is at most 2, at most 3, at most 4, at most 5, at most 6, at most 7, at most 8, at most 9, or at most 10 unique barcode bits. In some embodiments of any of the aspects, the total number of unique barcode bits is no more than 10 unique barcode bits.

In some embodiments of any of the aspects, the barcode-hybridizing region is unique to each readout molecule. In some embodiments of any of the aspects, the total number of unique barcode-hybridizing regions used in the set of readout molecules is less than the total number of unique barcode-hybridizing regions possible. As used herein, the phrase “total number of unique barcode-hybridizing regions possible” refers to the number of nucleotides and/or nucleotide analogs that can be used in the barcode-hybridizing region to the power of the number of nucleotide and/or nucleotide analog positions in a bit (i.e., the length of the region). As a non-limiting example, in a barcode-hybridizing region that is comprised of 4 different nucleotides and is 5 nucleotides long, the total number of unique barcode-hybridizing regions possible is 4\hat{ }5 or 4*4*4*4*4 or 1024.

In some embodiments of any of the aspects, the total number of unique barcode-hybridizing regions in the set of readout molecules is less than 10% of the total number of unique barcode-hybridizing regions possible. As a non-limiting example, in a barcode-hybridizing region that is comprised of 4 different nucleotides and is 5 nucleotides long, the total number of unique barcode-hybridizing regions is less than 100. In some embodiments of any of the aspects, the total number of unique barcode-hybridizing regions in the set of readout molecules is less than 1% of the total number of unique barcode-hybridizing regions possible. As a non-limiting example, in a barcode-hybridizing region that is comprised of 4 different nucleotides and is 5 nucleotides long, the total number of unique barcode-hybridizing regions is less than 10.

In some embodiments of any of the aspects, the total number of unique barcode-hybridizing regions in the set of readout molecules is at least 2 unique barcode-hybridizing regions. As a non-limiting example, the total number of unique barcode-hybridizing regions is at least 2, at least 3, at least 4, at least 5, at least 6, at least 7, at least 8, at least 9, or at least 10 unique barcode-hybridizing regions. In some embodiments of any of the aspects, the total number of unique barcode-hybridizing regions is no more than 10 unique barcode-hybridizing regions. As a non-limiting example, the total number of unique barcode-hybridizing regions is at most 2, at most 3, at most 4, at most 5, at most 6, at most 7, at most 8, at most 9, or at most 10 unique barcode-hybridizing regions. In some embodiments of any of the aspects, the total number of unique barcode-hybridizing regions is no more than 10 unique barcode-hybridizing regions.

In some embodiments of any of the aspects, the detecting step is performed with a sequencing method. In some embodiments of any of the aspects, the sequencing method comprises sequencing by ligation (SBL), sequencing by synthesis (SBS), sequencing by hybridization (SBH), and/or sequencing by cyclic reversible polymerization hybridization chain reaction. In some embodiments of any of the aspects, sequencing by ligation comprises enzyme-based ligation. In some embodiments of any of the aspects, sequencing by ligation comprises chemical ligation, copper assisted ligation, copper free click reaction, Amine-EDC based coupling, or thiol-maleimide Michael addition. See e.g., Shendure et al., Science 309 (5741): 1728-32; 2005; Guo et al., 2008, Proc Natl Acad Sci USA. 2008 Jul. 8, 105(27):9145-50; Lee et al. 2014, Science 343 (6177): 1360-1363; Chen et al. 2018, Nucleic Acids Research 46 (4): e22-e22; Wang et al. 2018, Science 361 (6400): eaat5691; patent publications WO 2013/055995, US 2014/0349294, WO 2008/151127; U.S. Pat. No. 8,481,258; the content of each of each is incorporated by reference herein in its entirety.

In some embodiments of any of the aspects, the detection method further comprises contacting the sample with at least one sequencing primer after contacting the sample with at least one oligonucleotide tag. In some embodiments of any of the aspects, the detection method further comprises contacting the sample with at least one sequencing primer prior to contacting the sample with a set of readout molecules.

In some embodiments of any of the aspects, after contacting the sample with a set of readout molecules (under conditions to allow for hybridization), the detection method further comprises ligating the 3′ end of the readout molecule to an adjacent nucleotide with a 5′ phosphate group. In some embodiments of any of the aspects, the optically detectable label is detected after the sample is contacted with a set of readout molecules. In some embodiments of any of the aspects, the optically detectable label or any region comprising an optically detectable label (e.g., the 5′ non-barcode-hybridizing region) is removed from the readout molecule through a cleavage step. In some embodiments of any of the aspects, the detection method further comprises at least one washing step, in between any step as described herein.

In some embodiments of any of the aspects, the steps of contacting with a set of readout molecules, ligating, detecting, and cleaving are repeated iteratively until the entire barcode region has been detected. In some embodiments of any of the aspects, the number of iterations of the steps of contacting with a set of readout molecules, ligating, detecting, and cleaving corresponds to the number of barcode bits in the barcode region of the oligonucleotide tag. As a non-limiting example, if a barcode region comprises 8 barcode bits, then the steps of contacting with a set of readout molecules, ligating, detecting, and cleaving are repeated iteratively 8 times.

Accordingly, in one aspect described herein is a method of detecting at least one target molecule in a sample, the method comprising: (a) contacting the sample with at least one oligonucleotide tag, each oligonucleotide tag comprising: (i) a recognition domain that binds specifically to a target molecule to be detected, and (ii) a street comprising a barcode region; (b) contacting the sample with at least one sequencing primer, wherein the sequencing primer hybridizes to at least one oligonucleotide tag; (c) contacting the sample with a set of readout molecules as described herein; (d) ligating the 3′ end of the readout molecule to a 5′ phosphate group (e.g., of the sequencing primer or another readout molecule); (e) detecting the optically detectable label; (f) cleaving the 5′ region of the readout molecule (e.g., comprising the optically detectable label); (g) repeating steps (c)-(f) until the entire barcode region has been detected; (h) detecting the relative order of the optically detectable labels hybridized to the at least one oligonucleotide tag, wherein the at least one oligonucleotide tag is hybridized to the at least one target molecule, whereby the relative order of the optically detectable labels permits identification of which oligonucleotide tag is hybridized to the target molecule at that location. See e.g., FIG. 10B or FIG. 10E.

In some embodiments of any of the aspects, the sample is contacted with a set of readout molecules, also referred to herein as a set, “readout set”, or “readout pool.” In some embodiments of any of the aspects, each readout pool is directed at determining the identity of a barcode bit at a specific position in the barcode region of an oligonucleotide tag. As a non-limiting example, the sample is sequentially or simultaneously contacted with at least one readout set to detect at least one barcode bit of the barcode region. As a non-limiting example, the sample is sequentially contacted with at least one readout set to detect a first and second barcode bit in one or more streets. In some embodiments of any of the aspects, the readout set comprises 2 readout molecules, 3 readout molecules, 4 readout molecules, or at least 5 readout molecules. In some embodiments of any of the aspects, the readout set comprises 2 distinct detectable labels, 3 distinct detectable labels, 4 distinct detectable labels, or at least 5 distinct detectable labels.

In some embodiments of any of the aspects, the readout set comprises a subset of readout molecules linked to the same type of detectable label. As a non-limiting example, the subset of readout molecules linked to the same type of detectable label comprise the same nucleotide at one position of the barcode-hybridizing region and at least one of: (1) different nucleotides at the other positions of the barcode-hybridizing region, (2) the set is degenerate at the other positions of the barcode-hybridizing region, or (3) universal nucleotides at the other positions of the barcode-hybridizing region. Universal nucleotides comprise universal bases that can bind to any nucleotide. Non-limiting examples of universal bases comprise inosine, deoxyinosine, hypoxanthine, nitroazoles, isocarbostyril analogues, azole carboxamides, or aromatic triazole analogues (see e.g., Loakes et al., Nucleic Acids Res. 2001 Jun. 15; 29(12):2437-47; Berger et al., Nucleic Acids Res. 2000 Aug. 1; 28(15): 2911-2914; Liang et al., RSC Advances 3(35); June 2013).

In some embodiments of any of the aspects, a readout set comprises at least 2 subsets of readout molecules, wherein each subset is linked to the same type of detectable label, which is distinct from the detectable label linked to the other subset(s) of readout molecules, and each subset detects the same barcode bit in the barcode region, which is distinct from the nucleotide in the same position of the barcode region detected by the other subset(s) of readout molecules. In some embodiments of any of the aspects, a readout set comprises 1 subset, 2 subsets, 3 subsets, 4 subsets, or at least 5 subsets of readout molecules.

In some embodiments of any of the aspects, the sample is contacted with a first readout set that recognizes the first bit of the barcode region of at least one oligonucleotide tag. In some embodiments of any of the aspects, the sample is contacted with a second readout set that recognizes the second bit of the barcode region of at least one oligonucleotide tag. In some embodiments of any of the aspects, the sample is contacted with a third readout set that recognizes the third bit of the barcode region of at least one oligonucleotide tag. In some embodiments of any of the aspects, the sample is contacted with a fourth readout set that recognizes the fourth bit of the barcode region of at least one oligonucleotide tag. In some embodiments of any of the aspects, the sample is contacted with a fifth readout set that recognizes the fifth bit of the barcode region of at least one oligonucleotide tag. In some embodiments of any of the aspects, the sample is contacted with a n^{th} readout set that recognizes the n^{th} bit of the barcode region of at least one oligonucleotide tag, where n corresponds to an integer from 1 to 10. In some embodiments of any of the aspects, each set of readout molecules is the same. In some embodiments of any of the aspects, each set of readout molecules is different from every other readout set. In some embodiments of any of the aspects, at least one set of readout molecules is different from every other readout set. In some embodiments of any of the aspects, at least one set of readout molecules is the same as at least one other readout set.

In some embodiments of any of the aspects, the sample is contacted with each readout set sequentially. In some embodiments of any of the aspects, in between contacting the sample with an n^{th} readout set and an (n+1)^{th} readout set, the readout set is detected as described herein, and the n^{th} readout set is washed away (e.g., with any buffer appropriate for use in hybridization reactions, e.g., 60% formamide in 2×SSCT, wherein SSC refers to saline-sodium citrate buffer and T refers to TWEEN).

In some embodiments of any of the aspects, the sample is contacted with at least two readout sets concurrently. As a non-limiting example, the sample is contacted concurrently with at least 2 readout sets, at least 3 readout sets, at least 4 readout sets, or at least 5 readout sets. Compared to contacting a sample with one readout set, contacting a sample with at least two readout sets concurrently can provide added benefits including but not limited to amplification of the signal, introduction of additional optically detectable markers (e.g., psuedocolor combinations of different fluorophores), and increased speed of the process.

In some embodiments of any of the aspects, the method of detecting at least one target molecule in a sample comprises contacting the sample with at least one oligonucleotide tag, contacting the sample with at least one readout sets, and detecting the relative spatial order of the readout molecules. In some embodiments of any of the aspects, the specific hybridization of a readout molecule to an oligonucleotide tag is determined by or is dependent on the identity of the barcode region.

In some embodiments of any of the aspects, the detecting is performed with at least single cell resolution. In some embodiments of any of the aspects, the detecting is performed with subcellular resolution. In some embodiments of any of the aspects, the detecting is performed with at least single nucleus resolution. As a non-limiting example, the detecting can be performed with a resolution of at least 200 nm, at least 300 nm, at least 400 nm, at least 500 nm, at least 600 nm, at least 700 nm, at least 800 nm, at least 900 nm, at least 1 m, at least 2 m, at least 3 m, at least 4 μm, at least 5 m, at least 6 m, at least 7 m, at least 8 m, at least 9 m, or at least 10 m. In some embodiments of any of the aspects, the detecting is performed with a resolution that can differentiate individual target molecules, e.g., chromosomes. In some embodiments of any of the aspects, the detecting is performed with super-resolution. As a non-limiting example, the detecting can be performed with a super-resolution of at least 10 nm, at least 20 nm, at least 30 nm, at least 40 nm, at least 50 nm, at least 60 nm, at least 70 nm, at least 80 nm, at least 90 nm, at least 100 nm, at least 110 nm, at least 120 nm, at least 130 nm, at least 140 nm, at least 150 nm, at least 160 nm, at least 170 nm, at least 180 nm, at least 190 nm, at least 200 nm, at least 210 nm, at least 220 nm, at least 230 nm, at least 240 nm, or at least 250 nm.

In one aspect described herein is an enhanced method of detecting at least one target molecule in a sample, the method comprising: (a) contacting the sample with at least one oligonucleotide tag, each oligonucleotide tag comprising: (i) a recognition domain that binds specifically to a target molecule to be detected, and (ii) a street comprising a barcode region that comprises at least one barcode bit; (b) contacting the sample with a readout molecule or set thereof as described herein (e.g., wherein at least one readout molecule comprises a nanoparticle, e.g., a metal nanoparticle, and/or wherein at least one readout molecule comprises an optically detectable label, e.g., a fluorophore); and (c) detecting the relative order of the optically detectable labels hybridized to the at least one oligonucleotide tag, wherein the at least one oligonucleotide tag is hybridized to the at least one target molecule, whereby the relative order of the optically detectable labels permits identification of which oligonucleotide tag is hybridized to the target molecule at that location. In some embodiments of any of the aspects, the method is enhanced by the nanoparticle (e.g., a metal nanoparticle) due to an overall signal amplification. Such signal amplification can result from multiple readout molecules linked to the nanoparticle (e.g., a metal nanoparticle) and/or Plasmon enhancement of fluorescence (e.g., between the nanoparticle, such as a metal nanoparticle, and the optically detectable label, such as a fluorophore).

In some embodiments of any of the aspects, the signal of the optically detectable label of the at least one readout molecule comprising a nanoparticle (e.g., a metal nanoparticle) is increased at least 1.5-fold compared to the signal of the optically detectable label of the same readout molecule not comprising the nanoparticle. In some embodiments of any of the aspects, the signal of the optically detectable label of the at least one readout molecule comprising a nanoparticle is increased at least 3-fold compared to the signal of the optically detectable label of the same readout molecule not comprising the nanoparticle. In some embodiments of any of the aspects, the signal of the optically detectable label of the at least one readout molecule comprising a nanoparticle is increased at least 10-fold compared to the signal of the optically detectable label of the same readout molecule not comprising the nanoparticle. In some embodiments of any of the aspects, the signal of the optically detectable label of the at least one readout molecule comprising a nanoparticle is increased at least 50-fold compared to the signal of the optically detectable label of the same readout molecule not comprising the nanoparticle. In some embodiments of any of the aspects, the signal of the optically detectable label of the at least one readout molecule comprising a nanoparticle is increased at least 1.1, 1.2, 1.3, 1.4, 1.5, 1.6, 1.7, 1.8, 1.9. 2, 3, 4, 5, 6, 7, 8, 9, 10, 15, 20, 25, 30, 35, 40, 45, 50, 55, 60, 65, 70, 75, 80, 85, 90, 95, or 100 fold compared to the signal of the optically detectable label of the same readout molecule not comprising the nanoparticle.

In one aspect, described herein is a method of karyotyping a biological sample, the method comprising: (a) contacting the sample with at least one oligonucleotide tag specific to at least one chromosome, each oligonucleotide tag comprising: (i) a recognition domain that binds specifically to a target molecule to be detected, and (ii) a street comprising a barcode region that comprises at least one barcode bit; (b) contacting the sample with a set of readout molecules as described herein; (c) detecting the relative order of the optically detectable labels hybridized to the at least one oligonucleotide tag, wherein the at least one oligonucleotide tag is hybridized to the at least one target molecule, whereby the relative order of the optically detectable labels permits identification of which oligonucleotide tag is hybridized to the target molecule at that location; and (d) determining the identity of at least one chromosome according to the identity of the least one oligonucleotide tag specific to the at least one chromosome.

In some embodiments of any of the aspects, the sample is contacted with at least one oligonucleotide tag specific to the p arm of the at least one chromosome. In some embodiments of any of the aspects, the sample is contacted with at least one oligonucleotide tag specific to the q arm of the at least one chromosome. In some embodiments of any of the aspects, the sample is contacted with at least one oligonucleotide tag specific to the p arm of the at least one chromosome, and at least one oligonucleotide tag specific to the q arm of the at least one chromosome. In some embodiments of any of the aspects, the sample is contacted with at least two oligonucleotide tags specific to the p arm or the q arm of the at least one chromosome. In some embodiments of any of the aspects, the sample is contacted with at least three oligonucleotide tags specific to the p arm or the q arm of the at least one chromosome.

In some embodiments of any of the aspects, the sample is contacted with at most 6 oligonucleotide tags specific to each chromosome arm. In some embodiments of any of the aspects, the sample is contacted with at most 10 oligonucleotide tags specific to each chromosome arm. In some embodiments of any of the aspects, the sample is contacted with at most 20 oligonucleotide tags specific to each chromosome arm. In some embodiments of any of the aspects, the sample is contacted with at most 5, at most 10, at most 15, at most 20, at most 25, at most 30, at most 35, at most 40, at most 45, at most 50, at most 55, at most 60, at most 65, at most 70, at most 75, at most 80, at most 85, at most 90, at most 95, or at most or 100 oligonucleotide tags specific to each chromosome arm.

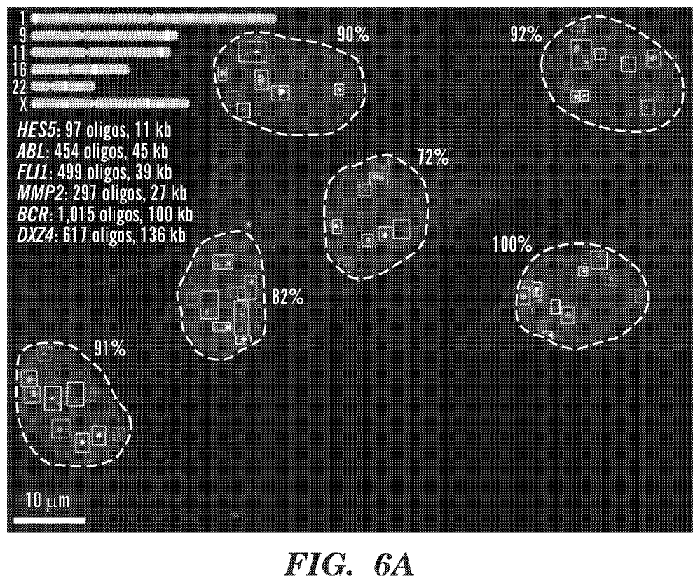

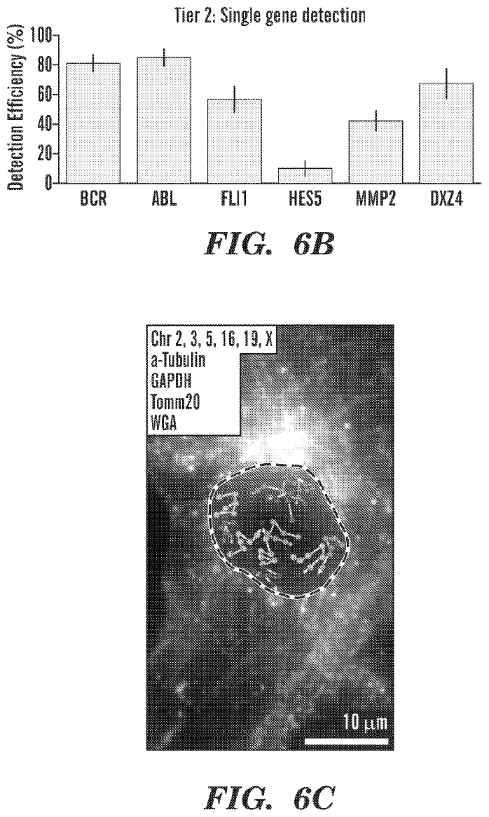

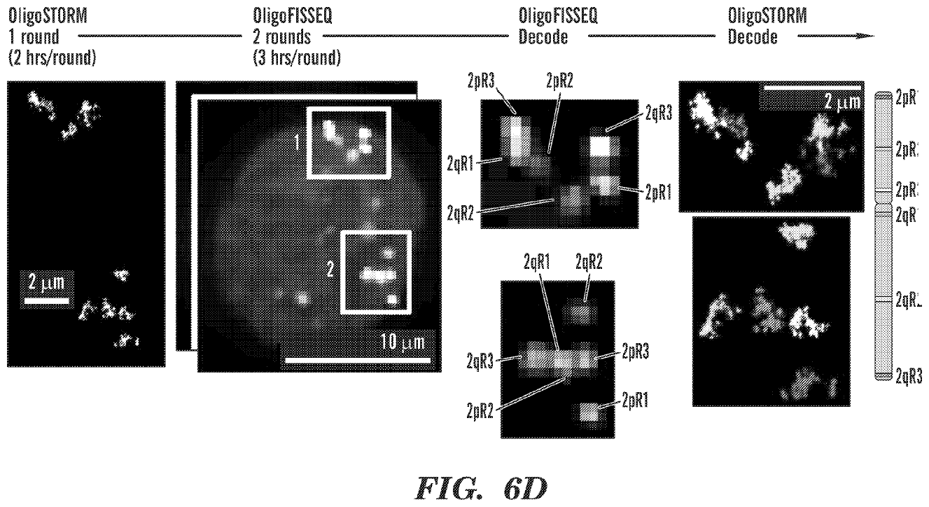

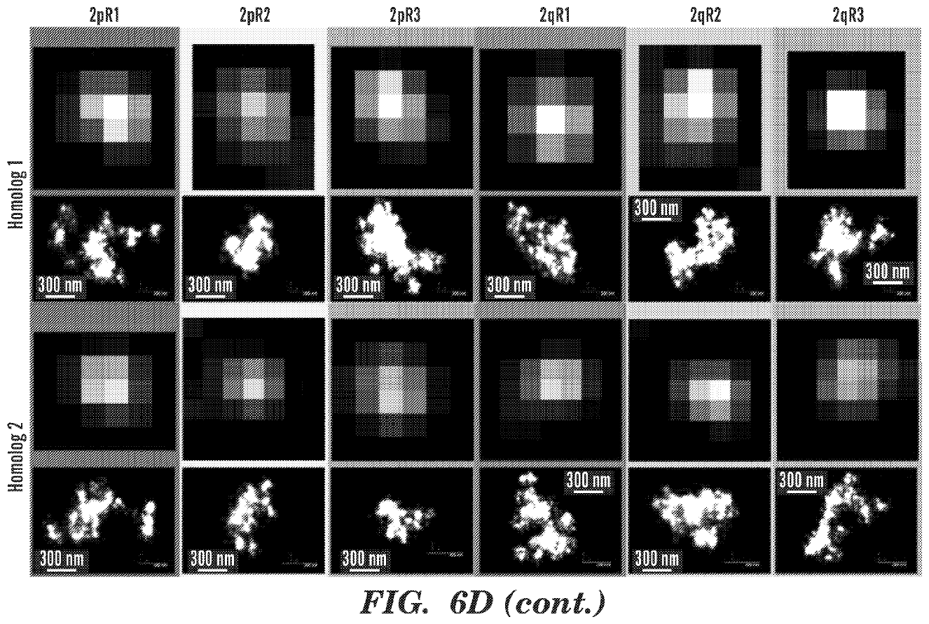

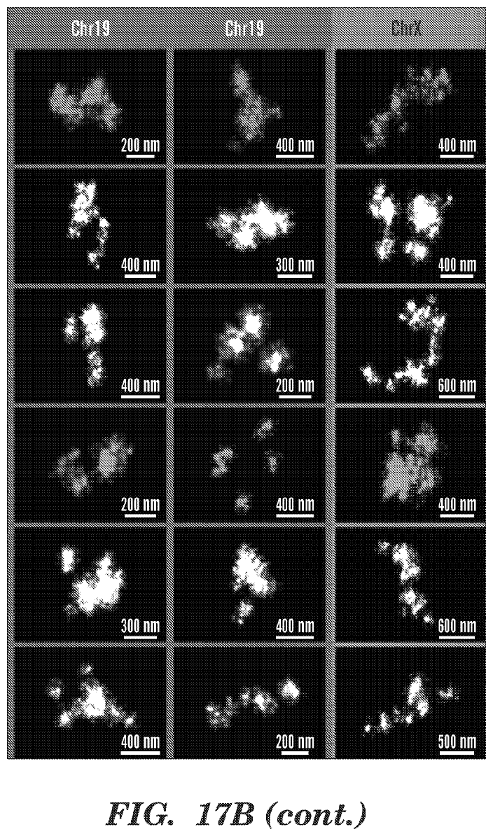

In some embodiments of any of the aspects, methods described herein can be performed sequentially or concurrently with additional methods, including but not limited to immunofluorescence (see e.g., FIG. 6C), or OligoSTORM (see e.g., FIG. 6D, FIG. 17A-17B).

In one aspect, described herein is a method of producing a high resolution image of at least one target molecule in a sample, the method comprising: (a) imaging the at least one target molecule using at least one round of a high resolution imaging method; and (b) determining the identity of the at least one imaged target molecule. In some embodiments of any of the aspects, the step of determining the identity of the at least one imaged target molecule comprises: (i) contacting the sample with at least one oligonucleotide tag, each oligonucleotide tag comprising: (A) a recognition domain that binds specifically to a target molecule to be detected, and (B) a street comprising a barcode region that comprises at least one barcode bit; (ii) contacting the sample with a set of readout molecules as described herein; and (iii) detecting the relative order of the optically detectable labels hybridized to the at least one oligonucleotide tag, wherein the at least one oligonucleotide tag is hybridized to the at least one target molecule, whereby the relative order of the optically detectable labels permits identification of which oligonucleotide tag is hybridized to the target molecule at that location.

In some embodiments of any of the aspects, the method comprises imaging at least 2 target molecules. In some embodiments of any of the aspects, the method comprises imaging at least 12 target molecules. In some embodiments of any of the aspects, the method comprises imaging at least 66 target molecules. In some embodiments of any of the aspects, the method comprises imaging at least 258 target molecules. In some embodiments of any of the aspects, the method comprises imaging at least 500 target molecules. In some embodiments of any of the aspects, the method comprises imaging at least 5000 target molecules.

In some embodiments of any of the aspects, the method comprises imaging at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, at least 400, at least 410, at least 420, at least 430, at least 440, at least 450, at least 460, at least 470, at least 480, at least 490, at least 500, at least 1000, at least 1500, at least 2000, at least 2500, at least 3000, at least 3500, at least 4000, at least 4500, or at least 5000 target molecules.

In some embodiments of any of the aspects, all of the target molecules are imaged at one time. In some embodiments of any of the aspects, at least half of the target molecules are imaged at one time. In some embodiments of any of the aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% of the target molecules are imaged at one time.

In some embodiments of any of the aspects, the method comprises at least two rounds of the high resolution imaging method. In some embodiments of any of the aspects, the method comprises at least three rounds of the high resolution imaging method. In some embodiments of any of the aspects, the method comprises at least five rounds of the high resolution imaging method. In some embodiments of any of the aspects, the method comprises at least 20 rounds of the high resolution imaging method. In some embodiments of any of the aspects, the method comprises at least 1 round, at least 2 rounds, at least 3 rounds, at least 4 rounds, at least 5 rounds, at least 10 rounds, at least 15 rounds, at least 20 rounds, at least 25 rounds, at least 30 rounds, at least 35 rounds, at least 40 rounds, at least 45 rounds, at least 50 rounds, at least 55 rounds, at least 60 rounds, at least 65 rounds, at least 70 rounds, at least 75 rounds, at least 80 rounds, at least 85 rounds, at least 90 rounds, at least 95 rounds, or at least 100 rounds of the high resolution imaging method.

In some embodiments of any of the aspects, the high resolution imaging method is selected from the group consisting of: Oligo Stochastic Optical Reconstruction Microscopy (OligoSTORM); structured illumination microscopy (SIM); Stimulated emission depletion (STED) microscopy; and Oligo DNA point accumulation in nanoscale topology (DNA-PAINT). In some embodiments of any of the aspects, the high resolution imaging method comprises Oligo Stochastic Optical Reconstruction Microscopy (OligoSTORM). In some embodiments of any of the aspects, the high resolution imaging method comprises structured illumination microscopy (SIM). In some embodiments of any of the aspects, the high resolution imaging method comprises Stimulated emission depletion (STED) microscopy. In some embodiments of any of the aspects, the high resolution imaging method comprises Oligo DNA point accumulation in nanoscale topology (DNA-PAINT). See e.g., Wu and Shroff, Faster, sharper, and deeper: structured illumination microscopy for biological imaging, Nature Methods 15, 1011-1019 (2018); Vicidomini et al. STED super-resolved microscopy. Nat Methods 15, 173-182 (2018); Beliveau et al. In situ super-resolution imaging of genomic DNA with OligoSTORM and OligoDNA-PAINT. Methods Mol Biol 2017 1663:231-252; the contents of each of which are incorporated herein by reference in their entireties.

In some embodiments of any of the aspects, detecting the relative order of the optically detectable labels hybridized to the at least one oligonucleotide tag comprises at least 2 rounds of contacting the sample with the set of readout molecules. In some embodiments of any of the aspects, detecting the relative order of the optically detectable labels hybridized to the at least one oligonucleotide tag comprises at least 3 rounds of contacting the sample with the set of readout molecules. In some embodiments of any of the aspects, detecting the relative order of the optically detectable labels hybridized to the at least one oligonucleotide tag comprises at least 5 rounds of contacting the sample with the set of readout molecules. In some embodiments of any of the aspects, detecting the relative order of the optically detectable labels hybridized to the at least one oligonucleotide tag comprises at least 10 rounds of contacting the sample with the set of readout molecules. In some embodiments of any of the aspects, detecting the relative order of the optically detectable labels hybridized to the at least one oligonucleotide tag comprises at least 20 rounds of contacting the sample with the set of readout molecules.

In some embodiments of any of the aspects, detecting the relative order of the optically detectable labels hybridized to the at least one oligonucleotide tag comprises at least 1 round, at least 2 rounds, at least 3 rounds, at least 4 rounds, at least 5 rounds, at least 10 rounds, at least 15 rounds, at least 20 rounds, at least 25 rounds, at least 30 rounds, at least 35 rounds, at least 40 rounds, at least 45 rounds, at least 50 rounds, at least 55 rounds, at least 60 rounds, at least 65 rounds, at least 70 rounds, at least 75 rounds, at least 80 rounds, at least 85 rounds, at least 90 rounds, at least 95 rounds, or at least 100 rounds of contacting the sample with the set of readout molecules.

In some embodiments of any of the aspects, compositions and methods described herein comprise improvements of compositions and methods related to Oligopaint technology. As used herein, the term “Oligopaint” refers to polynucleotides that have sequences complementary to a target molecule, e.g., an oligonucleotide sequence, a portion of a DNA sequence, or a particular chromosome or sub-chromosomal region of a particular chromosome.

Traditionally, fluorescence in situ hybridization (FISH) probes are derived from cloned genomic regions or flow-sorted chromosomes, which are labeled directly via nick translation or PCR in the presence of fluorophore-conjugated nucleotides or labeled indirectly with nucleotide-conjugated haptens, such as biotin and digoxigenin, and then visualized with secondary detection reagents. Traditional FISH probes are limited by repetitive sequences and variable efficacy. Furthermore, target regions are restricted by the availability of clones and the size of their genomic inserts. Whereas it is possible to target larger regions with traditional FISH probes, this approach is often challenging and expensive, as each clone needs to be prepared and optimized for hybridization separately.

Oligopaints are an improved FISH technology wherein oligo libraries can be produced by massively parallel synthesis can be used as a renewable source of probes. Oligo libraries can be PCR-amplified (optionally with fluorophore-conjugated primers). The amplification products can be enzymatically processed to produce highly efficient single-stranded, strand-specific probes that can visualize regions ranging from tens of kilobases to megabases. Oligopaints can comprise synthetic probes and arrays that are, optionally, computationally patterned and/or computationally designed.

For publications directed at Oligopaint and related technologies, see e.g., Beliveau et al. OligoMiner provides a rapid, flexible environment for the design of genome-scale oligonucleotide in situ hybridization probes. Proc. Nat. Acad. Sci. USA 2018 115:E2183-E2192; Beliveau et al. In situ super-resolution imaging of genomic DNA with OligoSTORM and OligoDNA-PAINT. Methods Mol Biol 2017 1663:231-252; Wang et al. Spatial organization of chromatin domains and compartments in single chromosomes. Science 2016 353:598-602; Boettiger et al. Super-resolution imaging reveals distinct chromatin folding for different epigenetic states. Nature. 2016 529:418-22; Schmidt et al. Scalable amplification of strand subsets from chip-synthesized oligonucleotide libraries. Nat Commun 2015 Nov. 16; 6:8634; Murgha et al. Combined in vitro transcription and reverse transcription method to amplify and label complex synthetic oligonucleotide probe libraries. BioTechniques 2015 58:301-7; Beliveau et al. Single-molecule super-resolution imaging of chromosomes and in situ haplotype visualization using Oligopaint FISH probes. Nat Commun 2015 6:7147; Beliveau et al. Visualizing genomes with Oligopaint FISH probes. Curr Protocols Mol Biol 2014 14.23; Beliveau et al. A versatile design and synthesis platform for visualizing genomes with Oligopaint FISH probes. Proc. Nat. Acad. Sci. USA 2012 109:21301-6; US 2010/0304994 A1; US 2018/0223347 A1; WO 2018/045186 A1; US 2014/0364333 A1; US 2019/0032121 A1; US 2013/0143208 A1; U.S. Pat. No. 10,119,160 B2; US 2018/0057867 A1; US 2019/0127786 A1; US 2018/0292318 A1; WO 2017/189525 A1; WO 2018/183851 A1; WO 2018/183860 A1; WO 2018/045181 A1; US 2016/0040235 A1; the content of each of which is incorporated herein by reference in its entirety.

As used herein, the terms “Oligopainted” and “Oligopainted region” refer to a target nucleotide sequence (e.g., a chromosome) or region of a target nucleotide sequence (e.g., a sub-chromosomal region), respectively, that has hybridized with one or more Oligopaints. Oligopaints can be used to label a target nucleotide sequence, e.g., chromosomes and sub-chromosomal regions of chromosomes during various phases of the cell cycle including, but not limited to, interphase, preprophase, prophase, prometaphase, metaphase, anaphase, telophase and cytokinesis.

Described herein are methods comprising OligoFISSEQ, i.e., Oligopaints comprising barcodes decoded with fluorescent in situ sequencing. OligoFISSEQ methods and compositions are described further in patent publications WO 2017/161251 and US 2019/0032121, the content of each of which are incorporated by reference herein in its entirety.

In some embodiments of any of the aspects, FISH methods can comprise Oligopaint, multiplexed error-robust fluorescence in situ hybridization (MERFISH), seqFISH, RNA sequential probing of targets (SPOTs), high-coverage microscopy-based technology (Hi-M), or optical reconstruction of chromatin architecture (ORCA) or any method comprising contacting a sample with a oligonucleotide that has a sequence complementary (e.g. recognition domain) to a target molecule, e.g., an oligonucleotide sequence, a portion of a DNA sequence, or a particular chromosome or sub-chromosomal region of a particular chromosome. For further details, see e.g., Cardozo et al., Mol Cell. 2019 Apr. 4; 74(1):212-222; Mateo et al., Nature. 2019 April; 568(7750):49-54; Wang et al., Scientific Reports volume 8, Article number: 4847 (2018); Shah et al., Neuron, Volume 92, Issue 2, 19 Oct. 2016, Pages 342-357; Eng et al., Nat Methods. 2017 Dec.; 14(12):1153-1155; each of which is incorporated herein by reference in its entirety.

In some embodiments of any of the aspects, the sets and methods described herein comprise detecting at least one target molecule in a sample. In some embodiments of any of the aspects, the target molecule comprises a nucleic acid, a polypeptide, a cell surface molecule, and/or an inorganic material. In some embodiments of any of the aspects, the target molecule comprises DNA, including but not limited to genomic DNA, genomic DNA organized as chromosomes, or complementary DNA (cDNA). In some embodiments of any of the aspects, the target molecule comprises RNA, including but not limited to messenger RNA (mRNA) or ribosomal RNA (rRNA). In some embodiments of any of the aspects, the target molecule is DNA and/or RNA. In some embodiments of any of the aspects, the target molecule comprises DNA and/or RNA. In some embodiments of any of the aspects, the target molecule consists of or consists essentially of DNA and/or RNA. In some embodiments of any of the aspects, the target molecule comprises a polypeptide.

In some embodiments of any of the aspects, the at least one target molecule comprises a 1 kb nucleic acid. In some embodiments of any of the aspects, the at least one target molecule comprises a 15 kb nucleic acid. In some embodiments of any of the aspects, the at least one target molecule comprises a 50 kb nucleic acid. In some embodiments of any of the aspects, the at least one target molecule comprises a 100 kb nucleic acid. In some embodiments of any of the aspects, the at least one target molecule comprises a 1 Mb nucleic acid. In some embodiments of any of the aspects, the at least one target molecule comprises a chromosome. In some embodiments of any of the aspects, the at least one target molecule comprises a genome.

In some embodiments of any of the aspects, the target molecule(s), oligonucleotide tag(s), readout molecule(s), and sequencing primer(s) can be any combination of DNA and RNA. As a non-limiting example, the target molecule(s), oligonucleotide tag(s), readout molecule(s), and sequencing primer(s) can all be DNA. As a non-limiting example, the target molecule(s), oligonucleotide tag(s), readout molecule(s), and sequencing primer(s) can all be RNA. As a non-limiting example, the target molecule(s) can be DNA; and the oligonucleotide tag(s), readout molecule(s), and sequencing primer(s) can be RNA. As a non-limiting example, the target molecule(s) can be RNA; and the oligonucleotide tag(s), readout molecule(s), and sequencing primer(s) can be DNA. Any other combinations of DNA and RNA can likewise be used. In some embodiments of any of the aspects, the target molecule(s), oligonucleotide tag(s), readout molecule(s), and/or sequencing primer(s) consist of or consist essentially of DNA and/or RNA.

In some embodiments of any of the aspects, the target molecule comprises a polypeptide, including but not limited to intracellular proteins, transmembrane proteins, or extracellular proteins. In some embodiments of any of the aspects, the target molecule comprises a cell surface molecule, including but not limited to transmembrane proteins, membrane lipids, membrane receptors, or transmembrane receptors. In some embodiments of any of the aspects, the target molecule comprises an inorganic material comprising any material derived from a non-living source, including but not limited to glass, ceramics, metals (e.g., circuit boards, surfaces comprising metal), or any other solid substrate. In some embodiments of any of the aspects, the target molecule is covalently or non-covalently linked to a nucleic acid, a polypeptide, a cell surface molecule, or an inorganic material. Non-limiting examples of such linkers and linking moieties are described further herein.

In some embodiments of any of the aspects, one target molecule is detected. In some embodiments of any of the aspects, at least two target molecules are detected concurrently. As a non-limiting example, at least 2 target molecules, at least 3 target molecules, at least 4 target molecules, at least 5 target molecules, at least 6 target molecules, at least 7 target molecules, at least 8 target molecules, at least 9 target molecules, at least 10 target molecules, at least 20 target molecules, at least 20 target molecules, at least 30 target molecules, at least 40 target molecules, at least 50 target molecules, at least 60 target molecules, at least 70 target molecules, at least 80 target molecules, at least 90 target molecules, or at least 100 target molecules are detected concurrently.

In some embodiments of any of the aspects, more than one region of a target molecule is detected concurrently. As a non-limiting example, at least 2 regions, at least 3 regions, at least 4 regions, at least 5 regions, at least 6 regions, at least 7 regions, at least 8 regions, at least 9 regions, at least 10 regions, at least 20 regions, at least 30 regions, at least 40 regions, at least 50 regions, at least 60 regions, at least 70 regions, at least 80 regions, at least 90 regions, or at least 100 regions, of a target molecule or target molecules are detected.

In some embodiments of any of the aspects, the sample is a cell, cell culture, or tissue sample. In some embodiments of any of the aspects, the sample comprises organoids (i.e., self-organized three-dimensional tissue cultures that are derived from stem cells). As a non-limiting example the cell, cell culture, or tissue sample is taken at a time or under conditions in which individual chromosomes are distinguishable, e.g., mitosis. In some embodiments of any of the aspects, the sample comprises a human cell nucleus. In some embodiments of any of the aspects, the sample comprises a nucleus from the cell of any organism. In some embodiments of any of the aspects, the sample comprises metaphase chromosomes. In some embodiments of any of the aspects, the sample comprises metaphase chromosome spreads. In some embodiments of any of the aspects, the metaphase chromosomes are obtained from a cultured cell nucleus. In some embodiments of any of the aspects, the metaphase chromosomes are obtained from a nucleus extracted from a tissue section, an organoid, or a biopsy specimen.

The term “sample” or “test sample” as used herein denotes a sample taken or isolated from a biological organism, e.g., a blood or tissue sample from a subject. In some embodiments of any of the aspects, the present invention encompasses several examples of a biological sample. In some embodiments of any of the aspects, the biological sample is cells, or tissue, or peripheral blood, or bodily fluid. Exemplary biological samples include, but are not limited to, a biopsy, a tumor sample, biofluid sample; blood; serum; plasma; urine; sperm; mucus; tissue biopsy; organ biopsy; synovial fluid; bile fluid; cerebrospinal fluid; mucosal secretion; effusion; sweat; saliva; and/or tissue sample etc. The term also includes a mixture of the above-mentioned samples. The term “test sample” also includes untreated or pretreated (or pre-processed) biological samples. In some embodiments of any of the aspects, a test sample comprises cells from a subject.

In some embodiments of any of the aspects, at least one target molecule is detected in at least one cell in a sample. In some embodiments of any of the aspects, at least one target molecule is detected concurrently in at least 2 cells in a sample. As a non-limiting sample, at least one target molecule is detected concurrently in at least 2 cells, at least 5 cells, at least 10 cells, at least 20 cells, at least 30 cells, at least 40 cells, at least 50 cells, at least 60 cells, at least 70 cells, at least 80 cells, at least 90 cells, at least 100 cells, at least 200 cells, at least 300 cells, at least 400 cells, at least 500 cells, at least 600 cells, at least 700 cells, at least 800 cells, at least 900 cells, at least 1,000 cells, at least 1,000 cells, at least 1,000 cells, at least 2,000 cells, at least 3,000 cells, at least 4,000 cells, at least 5,000 cells, at least 6,000 cells, at least 7,000 cells, at least 8,000 cells, at least 9,000 cells, or at least 10,000 cells in a sample.

The test sample can be obtained by removing a sample from a subject, but can also be accomplished by using a previously isolated sample (e.g. isolated at a prior time point and isolated by the same or another person).

In some embodiments of any of the aspects, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments of any of the aspects, the test sample can be a frozen test sample, e.g., a frozen tissue. The frozen sample can be thawed before employing methods, assays and systems described herein. After thawing, a frozen sample can be centrifuged before being subjected to methods, assays and systems described herein. In some embodiments of any of the aspects, the test sample is a clarified test sample, for example, by centrifugation and collection of a supernatant comprising the clarified test sample. In some embodiments of any of the aspects, a test sample can be a pre-processed test sample, for example, supernatant or filtrate resulting from a treatment selected from the group consisting of centrifugation, filtration, thawing, purification, and any combinations thereof. In some embodiments of any of the aspects, the test sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. The skilled artisan is well aware of methods and processes appropriate for pre-processing of biological samples required for determination of the level of an expression product as described herein.

In some embodiments of any of the aspects, the methods, assays, and systems described herein can further comprise a step of obtaining or having obtained a test sample from a subject. In some embodiments of any of the aspects, the subject can be a human subject.

Described herein are compositions (e.g., oligonucleotide tags, readout molecules, secondary oligonucleotides, primers, etc.) comprising nucleotides or analogs thereof. A nucleotide comprises a phosphate backbone, a pentose sugar (e.g., ribose, deoxyribose), and a nucleobase (e.g., adenine, cytosine, guanine, thymine, uracil). As used herein, the term “analog” (with reference to nucleotides, i.e., nucleotide analogs, nucleoside analogs, nucleic acid analogs, etc.) refers to a nucleotide-like composition comprising at least one modification in the phosphate backbone, pentose sugar, and/or nucleobase. Non-limiting examples of nucleotide analogs are described further herein, but nucleic acids as described herein (e.g., oligonucleotide tags, readout molecules, secondary oligonucleotides, and/or primers) can comprise any nucleotide analog known in the art.

In some embodiments of any of the aspects, a nucleic acid as described herein (e.g., oligonucleotide tags, readout molecules, secondary oligonucleotides, and/or primers) is chemically modified to enhance stability or other beneficial characteristics. The nucleic acids described herein may be synthesized and/or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of nucleic acid compounds useful in the embodiments described herein include, but are not limited to nucleic acids containing modified backbones or no natural internucleoside linkages. nucleic acids having modified backbones include, among others, those that do not have a phosphorus atom in the backbone. For the purposes of this specification, and as sometimes referenced in the art, modified nucleic acids that do not have a phosphorus atom in their internucleoside backbone can also be considered to be oligonucleosides. In some embodiments of any of the aspects, the modified nucleic acid will have a phosphorus atom in its internucleoside backbone.

Modified nucleic acid backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Modified nucleic acid backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; others having mixed N, O, S and CH2 component parts, and oligonucleosides with heteroatom backbones, and in particular —CH2-NH—CH2-, —CH2-N(CH3)-O—CH2-[known as a methylene (methylimino) or MMI backbone], —CH2-O—N(CH3)-CH2-, —CH2-N(CH3)-N(CH3)-CH2- and —N(CH3)-CH2-CH2-[wherein the native phosphodiester backbone is represented as —O—P—O—CH2-].

Modified nucleic acids can also contain one or more substituted sugar moieties. The nucleic acids described herein can include one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Exemplary suitable modifications include O[(CH2)nO] mCH3, O(CH2).nOCH3, O(CH2)nNH2, O(CH2) nCH3, O(CH2)nONH2, and O(CH2)nON[(CH2)nCH3)]2, where n and m are from 1 to about 10. In some embodiments of any of the aspects, dsRNAs include one of the following at the 2′ position: C1 to C10 lower alkyl, substituted lower alkyl, alkaryl, aralkyl, O-alkaryl or O-aralkyl, SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties a nucleic acid, or a group for improving the pharmacodynamic properties of a nucleic acid, and other substituents having similar properties. In some embodiments of any of the aspects, the modification includes a 2′ methoxyethoxy (2′-O—CH2CH20CH3, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH2)2ON(CH3)2 group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH2-O—CH2-N(CH2)2, also described in examples herein below.

Other modifications include 2′-methoxy (2′-OCH3), 2′-aminopropoxy (2′-OCH2CH2CH2NH2) and 2′-fluoro (2′-F). Similar modifications can also be made at other positions on the nucleic acid, particularly the 3′ position of the sugar on the 3′ terminal nucleotide or in 2′-5′ linked dsRNAs and the 5′ position of 5′ terminal nucleotide. Nucleic acids may also have sugar mimetics such as cyclobutyl moieties in place of the pentofuranosyl sugar.

A nucleic acid can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases can include the synthetic and natural nucleobases including but not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Certain of these nucleobases are particularly useful for increasing the binding affinity of the inhibitory nucleic acids featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications. In some embodiments of any of the aspects, modified nucleobases can include d5SICS and dNAM, which are a non-limiting example of unnatural nucleobases that can be used separately or together as base pairs (see e.g., Leconte et. al. J. Am. Chem. Soc.2008, 130, 7, 2336-2343; Malyshev et. al. PNAS. 2012. 109 (30) 12005-12010). In some embodiments of any of the aspects, nucleic acids as described herein (e.g., oligonucleotide tags, readout molecules, secondary oligonucleotides, and/or primers) comprise any modified nucleobases known in the art, i.e., any nucleobase that is modified from an unmodified and/or natural nucleobase.

The preparation of the modified nucleic acids, backbones, and nucleobases described above are well known in the art.

Another modification of a nucleic acid featured in the invention involves chemically linking to the nucleic acid to one or more ligands, moieties or conjugates that enhance the activity, cellular distribution, pharmacokinetic properties, or cellular uptake of the nucleic acid. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

In some embodiments of any of the aspects, each readout molecule comprises a cleavable modification. According to certain aspects of the present disclosure, cleavable nucleotide moieties also referred to as cleavable linkages or cleavable modifications are used to separate a barcode-hybridizing region from a non-barcode hybridizing region in a readout molecule. Cleavable moieties are known to those of skill in the art and include chemically scissile internucleosidic linkages which may be cleaved by treating them with chemicals or subjecting them to oxidizing or reducing environments. Such cleavable moieties include phosphorothioate, phosphorothiolate which can be cleaved by various metal ions such as solutions of silver nitrate. Such cleavable moieties include phosphoroamidate which can be cleaved in acidic conditions such as solutions including acetic acid. A suitable chemical that can cleave a linkage includes a chemical that can cleave a bridged-phosphorothioate linkage and can remove a phosphoramidite linker from a nucleotide and/or oligonucleotide, leaving a free phosphate group on the nucleotide and/or oligonucleotide at the cleavage site. Suitable chemicals include, but are not limited to AgNO3, AgCH3COO, AgBrO3, Ag2SO4, or any compound that delivers Ag2+, HgCl2, I2, Br2, I—, Br— and the like.

Cleavable moieties also include those that can be cleaved by nucleases known to those of skill in the art. Such nucleases include restriction endonucleases such as Type I, Type II, Type III and Type IV, endonucleases such as endonucleases I-VIII, ribonucleases and other nucleases such as enzymes with AP endonuclease activity, enzymes with AP lyase activity and enzymes with glycosylase activity such as uracil DNA glycosylase.

Cleavable moieties also include those capable of being cleaved by light of a certain wavelength. Such cleavable moieties are referred to as photolabile linkages and are disclosed in Olejnik et al., Photocleavable biotin derivatives: a versatile approach for the isolation of biomolecules, Proc. Natl. Acad. Sci. U.S.A., vol. 92, p. 7590-7594 (1995). Such photocleavable linkers can be cleaved by UV illumination between wavelengths of about 275 to about 375 nm for a period of a few seconds to 30 minutes, such as about one minute. Exemplary wavelengths include between about 300 nm to about 350 nm.

Certain nucleotides, such as dGTP, dCTP and dTTP could also be reacted before being incorporated for use as a cleavable linkage, making them specifically sensitive to further cleavage by nucleases or chemicals. According to one aspect, one or multiple deoxyguanosines in a given template non-hybridizing nucleic acid can be oxidized to 8-oxo-deoxyguanosine by 2-nitropropane, before being added to the detection (e.g., sequencing) reaction, and subsequently cleaved using an 8-oxoguanine DNA glycosylase (e.g. Fpg, hOGG1). Similarly, deoxycytosines can be pre-reacted to form 5-hydroxycytosine, using bisulfite or nitrous acid, which can then be processed by certain DNA-glycosylase, such as hNEIL1. Other nucleotides which can be cleaved include uracil, deoxyuridine, inosine and deoxyinosine.

Additional embodiments include nucleotides that may be cleaved in a two-step method such as by a first step that modifies the nucleotide making it more susceptible to cleavage and then a second step where the nucleotide is cleaved. Such systems include the USER system (commercially available from Enzymatics (#Y918L) or New England Biolabs (#M5505L) which is typically a combination of UDG and Endonuclease VIII, although other endonucleases could be used. Enzymes UDG and endonuclease are commercially available. In addition, modified nucleotides may be cleavable nucleotides where a feature of the nucleotide has been modified, such as a bond, so as to facilitate cleavage. Examples include an abasic base, an apyrimidic base, an apurinic base, phosphorothioate, phosphorothiolate and oxidized bases such as deoxyguanosines which can be oxidized to 8-oxo-deoxyguanosine.

Accordingly, internucleotide bonds may be cleaved by chemical, thermal, or light based cleavage. Exemplary chemically cleavable internucleotide linkages for use in the methods described herein include, for example, β-cyano ether, 5′-deoxy-5′-aminocarbamate, 3′deoxy-3′-aminocarbamate, urea, 2′cyano-3′,5′-phosphodiester, 3′-(S)-phosphorothioate, 5′-(S)-phosphorothioate, 3′-(N)-phosphoramidate, 5′-(N)-phosphoramidate, α-amino amide, vicinal diol, ribonucleoside insertion, 2′-amino-3′,5′-phosphodiester, allylic sulfoxide, ester, silyl ether, dithioacetal, 5′-thio-furmal, α-hydroxy-methyl-phosphonic bisamide, acetal, 3′-thio-furmal, methylphosphonate and phosphotriester. Internucleoside silyl groups such as trialkylsilyl ether and dialkoxysilane are cleaved by treatment with fluoride ion. Base-cleavable sites include 3-cyano ether, 5′-deoxy-5′-aminocarbamate, 3′-deoxy-3′-aminocarbamate, urea, 2′-cyano-3′,5′-phosphodiester, 2′-amino-3′,5′-phosphodiester, ester and ribose. Thio-containing internucleotide bonds such as 3′-(S)-phosphorothioate and 5′-(S)-phosphorothioate are cleaved by treatment with silver nitrate or mercuric chloride. Acid cleavable sites include 3′-(N)-phosphoramidate, 5′-(N)-phosphoramidate, dithioacetal, acetal and phosphonic bisamide. An α-aminoamide internucleoside bond is cleavable by treatment with isothiocyanate, and titanium may be used to cleave a 2′-amino-3′,5′-phosphodiester-O-ortho-benzyl internucleoside bond. Vicinal diol linkages are cleavable by treatment with periodate. Thermally cleavable groups include allylic sulfoxide and cyclohexene while photo-labile linkages include nitrobenzylether and thymidine dimer. Methods synthesizing and cleaving nucleic acids containing chemically cleavable, thermally cleavable, and photo-labile groups are described for example, in U.S. Pat. No. 5,700,642.

Accordingly, internucleotide bonds may be cleaved using enzymatic cleavage. Nucleic acid sequences described herein may be designed to include a restriction endonuclease cleavage site. A nucleic acid may be contacted with a restriction endonuclease to result in cleavage. A wide variety of restriction endonucleases having specific binding and/or cleavage sites are commercially available, for example, from New England Biolabs (Ipswich, Mass.). In various embodiments, restriction endonucleases that produce 3′ overhangs, 5′ overhangs or blunt ends may be used. When using a restriction endonuclease that produces an overhang, an exonuclease (e.g., RecJf, Exonuclease I, Exonuclease T, Si nuclease, P1 nuclease, mung bean nuclease, CEL I nuclease, etc.) may be used to produce blunt ends. In an exemplary embodiment, an orthogonal primer/primer binding site that contains a binding and/or cleavage site for a type IIS restriction endonuclease may be used to remove the temporary orthogonal primer binding site.

As used herein, the term “restriction endonuclease recognition site” is intended to include, but is not limited to, a particular nucleic acid sequence to which one or more restriction enzymes bind, resulting in cleavage of a DNA molecule either at the restriction endonuclease recognition sequence itself, or at a sequence distal to the restriction endonuclease recognition sequence. Restriction enzymes include, but are not limited to, type I enzymes, type II enzymes, type IIS enzymes, type III enzymes and type IV enzymes. The REBASE database provides a comprehensive database of information about restriction enzymes, DNA methyltransferases and related proteins involved in restriction-modification. It contains both published and unpublished work with information about restriction endonuclease recognition sites and restriction endonuclease cleavage sites, isoschizomers, commercial availability, crystal and sequence data (see Roberts et al. (2005) Nucl. Acids Res. 33:D230, incorporated herein by reference in its entirety for all purposes).

In certain aspects, primers of the present invention include one or more restriction endonuclease recognition sites that enable type IIS enzymes to cleave the nucleic acid several base pairs 3′ to the restriction endonuclease recognition sequence. As used herein, the term “type IIS” refers to a restriction enzyme that cuts at a site remote from its recognition sequence. Type IIS enzymes are known to cut at a distances from their recognition sites ranging from 0 to 20 base pairs. Examples of Type IIs endonucleases include, for example, enzymes that produce a 3′ overhang, such as, for example, Bsr I, Bsm I, BstF5 I, BsrD I, Bts I, Mnl I, BciV I, Hph I, Mbo II, Eci I, Acu I, Bpm I, Mme I, BsaX I, Bcg I, Bae I, Bfi I, TspDT I, TspGW I, Taq II, Eco57 I, Eco57M I, Gsu I, Ppi I, and Psr I; enzymes that produce a 5′ overhang such as, for example, BsmA I, Ple I, Fau I, Sap I, BspM I, SfaN I, Hga I, Bvb I, Fok I, BceA I, BsmF I, Ksp632 I, Eco31 I, Esp3 I, Aar I; and enzymes that produce a blunt end, such as, for example, Mly I and Btr I. Type-IIs endonucleases are commercially available and are well known in the art (New England Biolabs, Beverly, Mass.). Information about the recognition sites, cut sites and conditions for digestion using type IIs endonucleases may be found, for example, on the Worldwide web at neb.com/nebecomm/enzymefindersearch bytypeIIs.asp). Restriction endonuclease sequences and restriction enzymes are well known in the art and restriction enzymes are commercially available (New England Biolabs, Ipswich, Mass.).

According to certain aspects, the cleavable moiety may be within an oligonucleotide (e.g., a readout molecule) and may be introduced during in situ synthesis. A broad variety of cleavable moieties are available in the art of solid phase and microarray oligonucleotide synthesis (see e.g., Pon, R., Methods Mol. Biol. 20:465-496 (1993); Verma et al., Ann. Rev. Biochem. 67:99-134 (1998); U.S. Pat. Nos. 5,739,386, 5,700,642 and 5,830,655; and U.S. Patent Publication Nos. 2003/0186226 and 2004/0106728).

The cleavable site may be located along the oligonucleotide backbone, for example, a modified 3′-5′ internucleotide linkage in place of one of the phosphodiester groups, such as ribose, dialkoxysilane, phosphorothioate, and phosphoramidate internucleotide linkage. The cleavable oligonucleotide analogs may also include a substituent on, or replacement of, one of the bases or sugars, such as 7-deazaguanosine, 5-methylcytosine, inosine, uridine, and the like.

In one embodiment, cleavable sites contained within the modified oligonucleotide (e.g., readout molecule) may include chemically cleavable groups, such as dialkoxysilane, 3′-(S)-phosphorothioate, 5′-(S)-phosphorothioate, 3′-(N)-phosphoramidate, 5′-(N)phosphoramidate, and ribose. Synthesis and cleavage conditions of chemically cleavable oligonucleotides are described in U.S. Pat. Nos. 5,700,642 and 5,830,655. For example, depending upon the choice of cleavable site to be introduced, either a functionalized nucleoside or a modified nucleoside dimer may be first prepared, and then selectively introduced into a growing oligonucleotide fragment during the course of oligonucleotide synthesis. Selective cleavage of the dialkoxysilane may be effected by treatment with fluoride ion. Phosphorothioate internucleotide linkage may be selectively cleaved under mild oxidative conditions. Selective cleavage of the phosphoramidate bond may be carried out under mild acid conditions, such as 80% acetic acid. Selective cleavage of ribose may be carried out by treatment with dilute ammonium hydroxide.

In another embodiment, a non-cleavable hydroxyl linker may be converted into a cleavable linker by coupling a special phosphoramidite to the hydroxyl group prior to the phosphoramidite or H-phosphonate oligonucleotide synthesis as described in U.S. Patent Application Publication No. 2003/0186226. The cleavage of the chemical phosphorylation agent at the completion of the oligonucleotide synthesis yields an oligonucleotide (e.g., a readout molecule) bearing a phosphate group at the 3′ end. The 3′-phosphate end may be converted to a 3′ hydroxyl end by a treatment with a chemical or an enzyme, such as alkaline phosphatase, which is routinely carried out by those skilled in the art.

In another embodiment, the cleavable linking moiety may be a TOPS (two oligonucleotides per synthesis) linker (see e.g., PCT publication WO 93/20092). For example, the TOPS phosphoramidite may be used to convert a non-cleavable hydroxyl group on the solid support to a cleavable linker. A preferred embodiment of TOPS reagents is the Universal TOPS™ phosphoramidite. Conditions for Universal TOPS™ phosphoramidite preparation, coupling and cleavage are detailed, for example, in Hardy et al. Nucleic Acids Research 22(15):2998-3004 (1994). The Universal TOPS™ phosphoramidite yields a cyclic 3′ phosphate that may be removed under basic conditions, such as the extended ammonia and/or ammonia/methylamine treatment, resulting in the natural 3′ hydroxy oligonucleotide.

In another embodiment, a cleavable linking moiety may be an amino linker. The resulting oligonucleotides bound to the linker via a phosphoramidite linkage may be cleaved with 80% acetic acid yielding a 3′-phosphorylated oligonucleotide.

In another embodiment, the cleavable linking moiety may be a photocleavable linker, such as an ortho-nitrobenzyl photocleavable linker. Synthesis and cleavage conditions of photolabile oligonucleotides on solid supports are described, for example, in Venkatesan et al., J. Org. Chem. 61:525-529 (1996), Kahl et al., J. Org. Chem. 64:507-510 (1999), Kahl et al., J. Org. Chem. 63:4870-4871 (1998), Greenberg et al., J. Org. Chem. 59:746-753 (1994), Holmes et al., J. Org. Chem. 62:2370-2380 (1997), and U.S. Pat. No. 5,739,386. Ortho-nitrobenzyl-based linkers, such as hydroxymethyl, hydroxyethyl, and Fmoc-aminoethyl carboxylic acid linkers, may also be obtained commercially.

In some embodiments of any of the aspects, each readout molecule comprises an optically detectable label. In some embodiments of any of the aspects, measurement, and/or detection of a target molecule, e.g. a DNA target molecule, an RNA target molecule, or a polypeptide target molecule comprises contacting a sample obtained from a subject with a reagent or reagents as described herein. In some embodiments of any of the aspects, the reagent is detectably labeled. In some embodiments of any of the aspects, the reagent is capable of generating a detectable signal. In some embodiments of any of the aspects, the reagent generates a detectable signal when the target molecule is present.

In some embodiments of any of the aspects, one or more of the reagents described herein can comprise a detectable label and/or comprise the ability to generate a detectable signal (e.g. by catalyzing reaction converting a compound to a detectable product). Detectable labels can comprise, for example, a light-absorbing dye, a fluorescent dye, or a radioactive label. Detectable labels, methods of detecting them, and methods of incorporating them into reagents described herein are well known in the art.

In some embodiments of any of the aspects, detectable labels, molecules, and/or moieties can include those that can be detected by spectroscopic, photochemical, biochemical, immunochemical, electromagnetic, radiochemical, or chemical means, such as fluorescence, chemifluorescence, or chemiluminescence, or any other appropriate means. The detectable labels used in the methods described herein can be primary labels (where the label comprises a moiety that is directly detectable or that produces a directly detectable moiety) or secondary labels (where the detectable label binds to another moiety to produce a detectable signal, e.g., as is common in immunological labeling using secondary and tertiary antibodies). The detectable label can be linked by covalent or non-covalent means to the reagent. Alternatively, a detectable label can be linked such as by directly labeling a molecule that achieves binding to the reagent via a ligand-receptor binding pair arrangement or other such specific recognition molecules. Detectable labels can include, but are not limited to radioisotopes, bioluminescent compounds, chromophores, antibodies, chemiluminescent compounds, fluorescent compounds, metal chelates, and enzymes.

In other embodiments, the detection reagent is a label with a fluorescent compound. When the fluorescently labeled reagent is exposed to light of the proper wavelength, its presence can then be detected due to fluorescence. In some embodiments of any of the aspects, a detectable label can be a fluorescent dye molecule, or fluorophore including, but not limited to fluorescein, phycoerythrin, phycocyanin, o-phthalaldehyde, fluorescamine, Cy3™, Cy5™, allophycocyanin, Texas Red, peridinin chlorophyll, cyanine, tandem conjugates such as phycoerythrin-Cy5™, green fluorescent protein, rhodamine, fluorescein isothiocyanate (FITC) and Oregon Green™, rhodamine and derivatives (e.g., Texas red and tetrarhodimine isothiocyanate (TRITC)), biotin, phycoerythrin, AMCA, CyDyes™, 6-carboxyfhiorescein (commonly known by the abbreviations FAM and F), 6-carboxy-2′,4′,7′,4,7-hexachlorofiuorescein (HEX), 6-carboxy-4′,5′-dichloro-2′,7′-dimethoxyfiuorescein (JOE or J), N,N,N′,N′-tetramethyl-6carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g., umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc.; BODIPY dyes and quinoline dyes. In some embodiments of any of the aspects, a detectable label can be a radiolabel including, but not limited to ³H, ¹²⁵I, ³⁵S, ⁴C, ³²P, and ³³P. In some embodiments of any of the aspects, a detectable label can be an enzyme including, but not limited to horseradish peroxidase and alkaline phosphatase. An enzymatic label can produce, for example, a chemiluminescent signal, a color signal, or a fluorescent signal. Enzymes contemplated for use to detectably label an antibody reagent include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase. In some embodiments of any of the aspects, a detectable label is a chemiluminescent label, including, but not limited to lucigenin, luminol, luciferin, isoluminol, theromatic acridinium ester, imidazole, acridinium salt and oxalate ester. In some embodiments of any of the aspects, a detectable label can be a spectral colorimetric label including, but not limited to colloidal gold or colored glass or plastic (e.g., polystyrene, polypropylene, and latex) beads.

In some embodiments of any of the aspects, detection reagents can also be labeled with a detectable tag, such as c-Myc, HA, VSV-G, HSV, FLAG, V5, HIS, or biotin. Other detection systems can also be used, for example, a biotin-streptavidin system. In this system, the antibodies immunoreactive (i. e. specific for) with the biomarker of interest is biotinylated. Quantity of biotinylated antibody bound to the biomarker is determined using a streptavidin-peroxidase conjugate and a chromogenic substrate. Such streptavidin peroxidase detection kits are commercially available, e. g. from DAKO; Carpinteria, CA. A reagent can also be detectably labeled using fluorescence emitting metals such as ¹⁵²Eu, or others of the lanthanide series. These metals can be attached to the reagent using such metal chelating groups as diethylenetriaminepentaacetic acid (DTPA) or ethylenediaminetetraacetic acid (EDTA).

Detection method(s) used will depend on the particular detectable labels used in the readout molecules. In certain exemplary embodiments, chromosomes and/or chromosomal regions having one or more oligonucleotide tags (e.g., Oligopaint) and/or readout molecules bound thereto may be selected for and/or screened for using a microscope, a spectrophotometer, a tube luminometer or plate luminometer, x-ray film, a scintillator, a fluorescence activated cell sorting (FACS) apparatus, a microfluidics apparatus or the like.

In some embodiments of any of the aspects, the detectable labels comprise fluorophores or fluorescent compounds. Systems and devices for the measurement of fluorescence are well known in the art. Fluorescence measurement requires a light source that emits light comprising the appropriate absorption or excitation wavelength. The absorption or excitation wavelength of the compounds described herein is approximately 300-800 nm. In some embodiments of any of the aspects, the light source emits light comprising, consisting essentially of, or consisting of a wavelength of 300-870 nm. The light contacts the sample, which excites electrons in certain materials within the sample, also known as fluorophores, and causes the materials to emit light (light emission) in the form of fluorescence.

The system or device for measurement of fluorescence then detects the emitted light. In some embodiments, the system or device can comprise a filter or monochromator so that only light of desired wavelengths reaches the detector of the system or device. In some embodiments of any of the aspects, the system or device is configured to detect light comprising, consisting essentially of, or consisting of a wavelength of 300-800 nm. In some embodiments of any of the aspects, the system or device is configured to detect light comprising, consisting essentially of, or consisting of a wavelength of 300-800 nm. Suitable systems and devices are commercially available and can include, e.g., the 20/30 PV™ Microspectrometer or 508 PV™ Microscope Spectrometer from CRAIC (San Dimas, CA), the Duetta™, FluoroMax™, Fluorolog™, QuantaMaster 8000™, DeltaFlex™, DeltaPro, or Nanolog™ from Horiba (Irvine, CA), or the SP8 Lightning™, SP8 Falcon™, SP8 Dive™, TCS SPE™, HCS A™, or TCS SP8 X™ from Leica (Buffalo Grove, IL).

In some embodiments of any of the aspects, fluorescence photomicroscopy can be used to detect and record the results of in situ hybridization using routine methods known in the art. Alternatively, digital (computer implemented) fluorescence microscopy with image-processing capability may be used. Two well-known systems for imaging FISH of chromosomes having multiple colored labels bound thereto include multiplex-FISH (M-FISH) and spectral karyotyping (SKY). See Schrock et al. (1996) Science 273:494; Roberts et al. (1999) Genes Chrom. Cancer 25:241; Fransz et al. (2002) Proc. Natl. Acad. Sci. USA 99:14584; Bayani et al. (2004) Curr. Protocol. Cell Biol. 22.5.1-22.5.25; Danilova et al. (2008) Chromosoma 117:345; U.S. Pat. No. 6,066,459; and FISH TAG™ DNA Multicolor Kit instructions (Molecular probes) for a review of methods for painting chromosomes and detecting painted chromosomes.

In certain exemplary embodiments, images of fluorescently labeled chromosomes are detected and recorded using a computerized imaging system such as the Applied Imaging Corporation CytoVision™ System (Applied Imaging Corporation, Santa Clara, Calif) with modifications (e.g., software, Chroma 84000 filter set, and an enhanced filter wheel). Other suitable systems include a computerized imaging system using a cooled CCD camera (Photometrics, NU200 series equipped with Kodak™ KAF 1400 CCD) coupled to a Zeiss Axiophot™ microscope, with images processed as described by Ried et al. (1992) Proc. Natl. Acad. Sci. USA 89:1388). Other suitable imaging and analysis systems are described by Schrock et al., supra; and Speicher et al. (1996) Nature Genet. 12:368. In some embodiments of any of the aspects, the oligonucleotide tags (e.g., Oligopaint) are visualized with super resolution microscopy (e.g. Stochastic Optical Reconstruction Microscopy (STORM) Imaging).

The in situ hybridization methods described herein can be performed on a variety of biological or clinical samples, in cells that are in any (or all) stage(s) of the cell cycle (e.g., mitosis, meiosis, interphase, G0, G1, S and/or G2). Examples include all types of cell culture, animal or plant tissue, peripheral blood lymphocytes, buccal smears, touch preparations prepared from uncultured primary tumors, cancer cells, bone marrow, cells obtained from biopsy or cells in bodily fluids (e.g., blood, urine, sputum and the like), cells from amniotic fluid, cells from maternal blood (e.g., fetal cells), cells from testis and ovary, and the like. Samples are prepared for assays of the invention using conventional techniques, which typically depend on the source from which a sample or specimen is taken. These examples are not to be construed as limiting the sample types applicable to the methods and/or compositions described herein.

Hybridization of the oligonucleotide tags (e.g., Oligopaint) of the invention to target chromosomes sequences can be accomplished by standard in situ hybridization (ISH) techniques (see, e.g., Gall and Pardue (1981) Meth. Enzymol. 21:470; Henderson (1982) Int. Review of Cytology 76:1). Generally, ISH comprises the following major steps: (1) fixation of the biological structure to be detected (e.g., a chromosome spread), (2) pre-hybridization treatment of the biological structure to increase accessibility of target DNA (e.g., denaturation with heat or alkali), (3) optional pre-hybridization treatment to reduce nonspecific binding (e.g., by blocking the hybridization capacity of repetitive sequences), (4) hybridization of the mixture of nucleic acids to the nucleic acid in the biological structure or tissue; (5) post-hybridization washes to remove nucleic acid fragments not bound in the hybridization and (6) detection of the hybridized labelled oligonucleotides (e.g., hybridized oligonucleotide tags, e.g., Oligopaints). The reagents used in each of these steps and their conditions of use vary depending on the particular situation. For instance, step 3 will not always be necessary as the recognition domains described herein can be designed to avoid repetitive sequences). Hybridization conditions are also described in U.S. Pat. No. 5,447,841. It will be appreciated that numerous variations of in situ hybridization protocols and conditions are known and may be used in conjunction with the present invention by practitioners following the guidance provided herein.

As used herein, the term “hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid” or “duplex.” “Hybridization conditions” will typically include salt concentrations of less than about 1 M, more usually less than about 500 mM and even more usually less than about 200 mM. Hybridization temperatures can be as low as 5° C., but are typically greater than 22° C., more typically greater than about 30° C., and often in excess of about 37° C. Hybridizations are usually performed under stringent conditions, i.e., conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the Tm for the specific sequence at s defined ionic strength and pH. Exemplary stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM Na phosphate, 5 mM EDTA, pH 7.4) and a temperature of 25-30° C. are suitable for allele-specific probe hybridizations. For stringent conditions, see for example, Sambrook, Fritsche and Maniatis, Molecular Cloning A Laboratory Manual, 2nd Ed. Cold Spring Harbor Press (1989) and Anderson Nucleic Acid Hybridization, 1st Ed., BIOS Scientific Publishers Limited (1999). “Hybridizing specifically to” or “specifically hybridizing to” or like expressions refer to the binding, duplexing, or hybridizing of a molecule substantially to or only to a particular nucleotide sequence or sequences under stringent conditions when that sequence is present in a complex mixture (e.g., total cellular) DNA or RNA.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third non-target entity. A reagent specific for a given target is one that exhibits specific binding for that target under the conditions of the assay being utilized.

As used herein, the term “oligonucleotide” is intended to include, but is not limited to, a single-stranded DNA or RNA molecule, typically prepared by synthetic means. Nucleotides of the present invention will typically be the naturally-occurring nucleotides such as nucleotides derived from adenosine, guanosine, uridine, cytidine and thymidine. When oligonucleotides are referred to as “double-stranded,” it is understood by those of skill in the art that a pair of oligonucleotides exists in a hydrogen-bonded, helical array typically associated with, for example, DNA. In addition to the 100% complementary form of double-stranded oligonucleotides, the term “double-stranded” as used herein is also meant to include those form which include such structural features as bulges and loops (see Stryer, Biochemistry, Third Ed. (1988), incorporated herein by reference in its entirety for all purposes). As used herein, the term “polynucleotide” is intended to include, but is not limited to, two or more oligonucleotides joined together (e.g., by hybridization, ligation, polymerization and the like).

Nucleic acid and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Roiff, A et al. PCR: Clinical Diagnostics and Research, Springer (1994)).

In certain exemplary embodiments, universal primers can be used to amplify nucleic acid sequences such as, for example, oligonucleotide tags (e.g., Oligopaint). The term “universal primers” refers to a set of primers (e.g., a forward and reverse primer) that may be used for chain extension/amplification of a plurality of polynucleotides, e.g., the primers hybridize to sites that are common to a plurality of polynucleotides. For example, universal primers may be used for amplification of all, or essentially all, polynucleotides in a single pool. In some embodiments of any of the aspects, forward primers and reverse primers have the same sequence. In some embodiments of any of the aspects, the sequence of forward primers differs from the sequence of reverse primers. In still other aspects, a plurality of universal primers are provided, e.g., tens, hundreds, thousands or more.

In some embodiments of any of the aspects, the universal primers may be temporary primers that may be removed after amplification via enzymatic or chemical cleavage. In some embodiments of any of the aspects, the universal primers may be temporary primers that may be removed after amplification via enzymatic or chemical cleavage. In other embodiments, the universal primers may comprise a modification that becomes incorporated into the polynucleotide molecules upon chain extension. Exemplary modifications include, for example, a 3′ or 5′ end cap, a label (e.g., fluorescein), or a tag (e.g., a tag that facilitates immobilization or isolation of the polynucleotide, such as, biotin, etc.).

In some embodiments of any of the aspects, the methods disclosed herein comprise amplification of oligonucleotide sequences including, for example, oligonucleotide tags (e.g., Oligopaint). Amplification methods may comprise contacting a nucleic acid with one or more primers (e.g., universal primers) that specifically hybridize to the nucleic acid under conditions that facilitate hybridization and chain extension. Exemplary methods for amplifying nucleic acids include the polymerase chain reaction (PCR) (see, e.g., Mullis et al. (1986) Cold Spring Harb. Symp. Quant. Biol. 51 Pt 1:263 and Cleary et al. (2004) Nature Methods 1:241; and U.S. Pat. Nos. 4,683,195 and 4,683,202), anchor PCR, RACE PCR, ligation chain reaction (LCR) (see, e.g., Landegran et al. (1988) Science 241:1077-1080; and Nakazawa et al. (1994) Proc. Natl. Acad. Sci. U.S.A. 91:360-364), self-sustained sequence replication (Guatelli et al. (1990) Proc. Natl. Acad. Sci. U.S.A. 87:1874), transcriptional amplification system (Kwoh et al. (1989) Proc. Natl. Acad. Sci. U.S.A. 86:1173), Q-Beta Replicase (Lizardi et al. (1988) BioTechnology 6:1197), recursive PCR (Jaffe et al. (2000) J. Biol. Chem. 275:2619; and Williams et al. (2002) J. Biol. Chem. 277:7790), the amplification methods described in U.S. Pat. Nos. 6,391,544, 6,365,375, 6,294,323, 6,261,797, 6,124,090 and 5,612,199, or any other nucleic acid amplification method using techniques well known to those of skill in the art. In exemplary embodiments, the methods disclosed herein utilize PCR amplification.

In general, the PCR procedure describes a method of gene amplification which is comprised of (i) sequence-specific hybridization of primers to specific genes or sequences within a nucleic acid sample or library, (ii) subsequent amplification involving multiple rounds of annealing, elongation, and denaturation using a thermostable DNA polymerase, and (iii) screening the PCR products for a band of the correct size. The primers used are oligonucleotides of sufficient length and appropriate sequence to provide initiation of polymerization, i.e. each primer is specifically designed to be complementary to a strand of the genomic locus to be amplified. In an alternative embodiment, mRNA level of gene expression products described herein can be determined by reverse-transcription (RT) PCR and by quantitative RT-PCR (QRT-PCR) or real-time PCR methods. Methods of RT-PCR and QRT-PCR are well known in the art.

In some embodiments of any of the aspects, the oligonucleotide tags (e.g., an Oligopaint) are not necessarily amplified (e.g., through PCR and/or universal priming regions). In some embodiments of any of the aspects, the oligonucleotide tags (e.g., an Oligopaint) described can be synthesized, de novo, and used “straight from the tube”. Methods of synthesizing oligonucleotides de novo are well known to those of skill in the art. As used herein, “oligonucleotide synthesis” refers to the chemical synthesis of relatively short fragments of nucleic acids with defined chemical structure. As a non-limiting example, methods of oligonucleotide synthesis include phosphoramidite solid-phase synthesis, phosphoramidite synthesis, phosphodiester synthesis, phosphotriester synthesis, or phosphite triester synthesis. See e.g., Beaucage et al. Tetrahedron Volume 48, Issue 12, 20 Mar. 1992, Pages 2223-2311; Caruthers, J Biol Chem. 2013 Jan. 11, 288(2):1420-7. In some embodiments, each oligonucleotide is synthesized separately. In some embodiments, the entire oligonucleotide set is synthesized in one reaction. In some embodiments, a subset of the entire oligonucleotide set is synthesized in one reaction. In some embodiments, the entire oligonucleotide set is synthesized in multiple, separate reactions. In some embodiments, reaction products are isolated, e.g., by high-performance liquid chromatography (HPLC), to obtain the desired oligonucleotides in high purity.

In certain exemplary embodiments, kits are provided. As used herein, the term “kit” refers to any delivery system for delivering oligonucleotide tags (e.g., an Oligopaint), readout molecules, primers, and/or reagents (e.g., ligase, a cleaving agent) for carrying out a method described herein. In the context of assays, such kits include systems that allow for the storage, transport, or delivery of reaction reagents (e.g., an enclosure providing one or more of, e.g., oligonucleotide tags, readout molecules, primers (e.g., primers specific for all oligonucleotide tags present and/or one or more subsets of primers specific to one or more subsets of oligonucleotide tag sequences), oligonucleotides having one or more detectable and/or retrievable labels bound thereto), supports having oligonucleotides bound thereto (e.g., microarrays, palettes, etc.), or the like) and/or supporting materials (e.g., an enclosure providing, e.g., buffers, written instructions for performing an assay described herein, or the like) from one location to another. For example, kits include one or more enclosures (e.g., boxes) containing the relevant reaction reagents and/or supporting materials for assays described herein. In one aspect, kits of the invention comprise oligonucleotide tags (e.g., an Oligopaint) specific for one or more target nucleotide sequences (e.g., chromosomes) or one or more regions of one or more target nucleotide sequences (e.g., sub-chromosomal regions). In one aspect, kits of the invention comprise readout molecules specific for one or more oligonucleotide tags (e.g., an Oligopaint). In another aspect, kits comprise one or more primer sequences, one or more supports having a plurality of synthetic, oligonucleotide sequences attached thereto, and one or more detectable and/or retrievable labels. Such contents may be delivered to the intended recipient together or separately. For example, a first container may contain primer sequences for use in an assay, while a second container may contain a support having a plurality of synthetic, oligonucleotide sequences attached thereto.

In some embodiments of any of the aspects, a kit provides one or more arrays and/or palettes having a plurality of specific oligonucleotide sequences (e.g., oligonucleotide tags (e.g., an Oligopaint) and/or readout molecules) bound thereto. In some embodiments of any of the aspects, an array and/or palette provides a plurality of oligonucleotide tag sequences (e.g., Oligopaints) that is specific for a set of binding patterns in a genome (e.g., a human genome). In some embodiments of any of the aspects, an array or palette is specific for a set of chromosomal aberrations (e.g., one or more of a translocation, an insertion, an inversion, a deletion, a duplication, a transposition, aneuploidy, polyploidy, complex rearrangement and telomere loss) associated with one or more disorders described herein. In some embodiments of any of the aspects, the kits described herein are particularly suited for diagnostic and/or prognostic use for detecting one or more disorders described herein in clinical settings (e.g., hospitals, medical clinics, medical offices, diagnostic laboratories, research laboratories and the like (e.g., for patient diagnosis and/or prognosis, prenatal diagnosis and/or prognosis and the like).

In some embodiments of any of the aspects, a kit provides instructions for amplifying the plurality of specific oligonucleotide tag sequences (e.g., Oligopaints) provided in the kit. In some embodiments of any of the aspects, the kit provides instructions for detectably and/or retrievably labeling one or more target nucleic acid sequences (e.g., one or more chromosomes or sub-chromosomal regions) using the amplified oligonucleotide tags (e.g., an Oligopaint). In some embodiments of any of the aspects, the kit provides instructions for detectably and/or retrievably labeling one or more target nucleic acid sequences (e.g., one or more chromosomes or sub-chromosomal regions) using the oligonucleotide tags (e.g., an Oligopaint) and readout molecules. In some embodiments of any of the aspects, a kit provides instructions for effectively removing one or more of the plurality of specific oligonucleotide tag sequences (e.g., Oligopaints) during the amplification step by including one or more unlabeled amplification primers that hybridizes to the one or more oligonucleotide sequences that one wishes to remove, such that the one or more target nucleic acid sequences is rendered not detectably and/or retrievably labeled.

In some embodiments of any of the aspects, systems and methods described herein may be implemented with any type of hardware and/or software, and may be a pre-programmed general purpose computing device. For example, the system may be implemented using a server, a personal computer, a portable computer, a thin client, or any suitable device or devices. The disclosure and/or components thereof may be a single device at a single location, or multiple devices at a single, or multiple, locations that are connected together using any appropriate communication protocols over any communication medium such as electric cable, fiber optic cable, or in a wireless manner.

It should also be noted that the disclosure is illustrated and discussed herein as having a plurality of modules which perform particular functions. It should be understood that these modules are merely schematically illustrated based on their function for clarity purposes only, and do not necessary represent specific hardware or software. In this regard, these modules may be hardware and/or software implemented to substantially perform the particular functions discussed. Moreover, the modules may be combined together within the disclosure, or divided into additional modules based on the particular function desired. Thus, the disclosure should not be construed to limit the present invention, but merely be understood to illustrate one example implementation thereof.

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

The operations described in this specification can be implemented as operations performed by a “data processing apparatus” on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a standalone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of nonvolatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

As used herein, the term “chromosome” refers to the support for the genes carrying heredity in a living cell, including DNA, protein, RNA and other associated factors. The conventional international system for identifying and numbering the chromosomes of the human genome is used herein. The size of an individual chromosome may vary within a multi-chromosomal genome and from one genome to another. A chromosome can be obtained from any species. A chromosome can be obtained from an adult subject, a juvenile subject, an infant subject, from an unborn subject (e.g., from a fetus, e.g., via prenatal test such as amniocentesis, chorionic villus sampling, and the like or directly from the fetus, e.g., during a fetal surgery) from a biological sample (e.g., a biological tissue, fluid or cells (e.g., sputum, blood, blood cells, tissue or fine needle biopsy samples, urine, cerebrospinal fluid, peritoneal fluid, and pleural fluid, or cells therefrom) or from a cell culture sample (e.g., primary cells, immortalized cells, partially immortalized cells or the like). In certain exemplary embodiments, one or more chromosomes can be obtained from one or more genera including, but not limited to, Homo, Drosophila, Caenorhabiditis, Danio, Cyprinus, Equus, Canis, Ovis, Ocorynchus, Salmo, Bos, Sus, Gallus, Solanum, Triticum, Oryza, Zea, Hordeum, Musa, Avena, Populus, Brassica, Saccharum and the like.

As used herein, the term “chromosome banding” refers to differential staining of chromosomes resulting in a pattern of transverse bands of distinguishable (e.g., differently or alternately colored) regions, that is characteristic for the individual chromosome or chromosome region (i.e., the “banding pattern”). Conventional banding techniques include G-banding (Giemsa stain), Q-banding (Quinacrine mustard stain), R-banding (reverse-Giemsa), and C-banding (centromere banding).

As used herein, the term “karyotype” refers to the chromosome characteristics of an individual cell, cell line or genome of a given species, as defined by both the number and morphology of the chromosomes. Karyotype can refer to a variety of chromosomal rearrangements including, but not limited to, translocations, insertional translocations, inversions, deletions, duplications, transpositions, aneuploidies, complex rearrangements, telomere loss and the like. Typically, the karyotype is presented as a systematized array of prophase or metaphase (or otherwise condensed) chromosomes from a photomicrograph or computer-generated image. Interphase chromosomes may also be examined.

As used herein, the terms “chromosomal aberration” or “chromosome abnormality” refer to a deviation between the structure of the subject chromosome or karyotype and a normal (i.e., non-aberrant) homologous chromosome or karyotype. The deviation may be of a single base pair or of many base pairs. The terms “normal” or “non-aberrant,” when referring to chromosomes or karyotypes, refer to the karyotype or banding pattern found in healthy individuals of a particular species and gender. Chromosome abnormalities can be numerical or structural in nature, and include, but are not limited to, aneuploidy, polyploidy, inversion, translocation, deletion, duplication and the like. Chromosome abnormalities may be correlated with the presence of a pathological condition or with a predisposition to developing a pathological condition. Chromosome aberrations and/or abnormalities can also refer to changes that are not associated with a disease, disorder and/or a phenotypic change. Such aberrations and/or abnormalities can be rare or present at a low frequency (e.g., a few percent of the population (e.g., polymorphic)).

Disorders associated with one or more chromosome abnormalities include, but are not limited to: autosomal abnormalities (e.g., trisomies (Down syndrome (chromosome 21), Edwards syndrome (chromosome 18), Patau syndrome (chromosome 13), trisomy 9, Warkany syndrome (chromosome 8), trisomy 22/cat eye syndrome, trisomy 16); monosomies and/or deletions (Wolf-Hirschhorn syndrome (chromosome 4), Cri du chat/Chromosome 5q deletion syndrome (chromosome 5), Williams syndrome (chromosome 7), Jacobsen syndrome (chromosome 11), Miller-Dieker syndrome/Smith-Magenis syndrome (chromosome 17), Di George's syndrome (chromosome 22), genomic imprinting (Angelman syndrome/Prader-Willi syndrome (chromosome 15))); X/Y-linked abnormalities (e.g., monosomies (Turner syndrome (XO), trisomy or tetrasomy and/or other karyotypes or mosaics (Klinefelter's syndrome (47 (XXY)), 48 (XXYY), 48 (XXXY), 49 (XXXYY), 49 (XXXXY), Triple X syndrome (47 (XXX)), 48 (XXXX), 49 (XXXXX), 47 (XYY), 48 (XYYY), 49 (XYYYY), 46 (XX/XY)); translocations (e.g., leukemia or lymphoma (e.g., lymphoid (e.g., Burkitt's lymphoma t(8 MYC; 14 IGH), follicular lymphoma t(14 IGH; 18 BCL2), mantle cell lymphoma/multiple myeloma t(11 CCND1; 14 IGH), anaplastic large cell lymphoma t(2 ALK; 5 NPM1), acute lymphoblastic leukemia) or myeloid (e.g., Philadelphia chromosome t(9 ABL; 22 BCR), acute myeloblastic leukemia with maturation t(8 RUNX1T1;21 RUNX1), acute promyelocytic leukemia t(15 PML,17 RARA), acute megakaryoblastic leukemia t(1 RBM15;22 MKL1))) or other (e.g., Ewing's sarcoma t(11 Fill; 22 EWS), synovial sarcomat(x SYT;18 SSX), dermatofibrosarcoma protuberans t(17 COL1A1; 22 PDGFB), myxoid liposarcoma t(12 DDIT3; 16 FUS), desmoplastic small round cell tumor t(11 WT1; 22 EWS), alveolar rhabdomyosarcoma t(2 PAX3; 13 FOXO1) t (1 PAX7; 13 FOXO1))); gonadal dysgenesis (e.g., mixed gonadal dysgenesis, XX gonadal dysgenesis); and other abnormalities (e.g., fragile X syndrome, uniparental disomy). Disorders associated with one or more chromosome abnormalities also include, but are not limited to, Beckwith-Wiedmann syndrome, branchio-oto-renal syndrome, Cri-du-Chat syndrome, De Lange syndrome, holoprosencephaly, Rubinstein-Taybi syndrome and WAGR syndrome.

Disorders associated with one or more chromosome abnormalities also include cellular proliferative disorders (e.g., cancer). As used herein, the term “cellular proliferative disorder” includes disorders characterized by undesirable or inappropriate proliferation of one or more subset(s) of cells in a multicellular organism. The term “cancer” refers to various types of malignant neoplasms, most of which can invade surrounding tissues, and may metastasize to different sites (see, for example, PDR Medical Dictionary 1st edition, 1995). The terms “neoplasm” and “tumor” refer to an abnormal tissue that grows by cellular proliferation more rapidly than normal and continues to grow after the stimuli that initiated proliferation is removed (see, for example, PDR Medical Dictionary 1st edition, 1995). Such abnormal tissue shows partial or complete lack of structural organization and functional coordination with the normal tissue which may be either benign (i.e., benign tumor) or malignant (i.e., malignant tumor).

Disorders associated with one or more chromosome abnormalities also include brain disorders including, but not limited to, acoustic neuroma, acquired brain injury, Alzheimer's disease, amyotrophic lateral diseases, aneurism, aphasia, arteriovenous malformation, attention deficit hyperactivity disorder, autism Batten disease, Bechet's disease, blepharospasm, brain tumor, cerebral palsy Charcot-Marie-Tooth disease, chiari malformation, CIDP, non-Alzheimer-type dementia, dysautonomia, dyslexia, dysprazia, dystonia, epilepsy, essential tremor, Friedrich's ataxia, gaucher disease, Gullian-Barre syndrome, headache, migraine, Huntington's disease, hydrocephalus, Meniere's disease, motor neuron disease, multiple sclerosis, muscular dystrophy, myasthenia gravis, narcolepsy, Parkinson's disease, peripheral neuropathy, progressive supranuclear palsy, restless legs syndrome, Rett syndrome, schizophrenia, Shy Drager syndrome, stroke, subarachnoid hemorrhage, Sydenham's syndrome, Tay-Sachs disease, Tourette syndrome, transient ischemic attack, transverse myelitis, trigeminal neuralgia, tuberous sclerosis and von Hippel-Lindau syndrome.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

In some embodiments of any of the aspects, the reference sample or level is the sample or level of the sample itself prior to being contacted with a composition described herein. In some embodiments of any of the aspects, the reference sample or level is the sample or level of a composition described herein prior to being contacted with the sample. In some embodiments of any of the aspects, the reference can be a sample contacted with compositions not comprising detectable labels. In some embodiments of any of the aspects, the reference can be a sample contacted with compositions comprising recognition domains that are not specific to the sample. In some embodiments of any of the aspects, the reference can also be a level obtained from a control sample, a pooled sample of control individuals, or a numeric value or range of values based on the same.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wild-type reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In some embodiments of any of the aspects, a single-stranded nucleic acid is produced by in-vitro transcription followed by reverse transcription. In some embodiments of any of the aspects, a single-stranded nucleic acid is produced by exposure to nicking endonuclease. In some embodiments of any of the aspects, a single-stranded nucleic acid is synthesized de novo. In one aspect, the nucleic acid can be DNA. In another aspect, the nucleic acid can be RNA. Suitable DNA can include, e.g., genomic DNA or cDNA. Suitable RNA can include, e.g., mRNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

In some embodiments, the expression of a biomarker(s), target(s), or gene/polypeptide described herein is/are tissue-specific. In some embodiments, the expression of a biomarker(s), target(s), or gene/polypeptide described herein is/are global. In some embodiments, the expression of a biomarker(s), target(s), or gene/polypeptide described herein is systemic.

“Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

“Marker” in the context of the present invention refers to an expression product, e.g., nucleic acid or polypeptide which is differentially present in a sample taken from a test subject, as compared to a comparable sample taken from control subjects (e.g., a healthy subject). The term “biomarker” is used interchangeably with the term “marker.”

In some embodiments, the methods described herein relate to measuring, detecting, or determining the level of at least one target molecule. As used herein, the term “detecting” or “measuring” refers to observing a signal from, e.g. a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some embodiments of any of the aspects, measuring can be a quantitative observation.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

In some embodiments of any of the aspects, the nucleic acid (e.g., oligonucleotide tag, readout molecules) described herein is exogenous. In some embodiments of any of the aspects, the nucleic acid (e.g., oligonucleotide tag, readout molecules) described herein is ectopic. In some embodiments of any of the aspects, the nucleic acid (e.g., oligonucleotide tag, readout molecules) described herein is not endogenous.

The term “exogenous” refers to a substance present in a cell other than its native source. The term “exogenous” when used herein can refer to a nucleic acid (e.g. a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes substance, such as a polypeptide or nucleic acid that is not naturally found or expressed in a given cell in its natural environment.

In some embodiments, a nucleic acid encoding a nucleic acid (e.g. an oligonucleotide tag, a readout molecule) or polypeptide as described herein is comprised by a vector. In some of the aspects described herein, a nucleic acid sequence encoding a given nucleic acid or polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments of any of the aspects, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments of any of the aspects, the vector or nucleic acid described herein is codon-optimized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments of any of the aspects, the expression system is an organism other than the source of the native/wild-type sequence (or a cell obtained from such organism). In some embodiments of any of the aspects, the vector and/or nucleic acid sequence described herein is codon-optimized for expression in a mammal or mammalian cell, e.g., a mouse, a murine cell, or a human cell. In some embodiments of any of the aspects, the vector and/or nucleic acid sequence described herein is codon-optimized for expression in a human cell. In some embodiments of any of the aspects, the vector and/or nucleic acid sequence described herein is codon-optimized for expression in a yeast or yeast cell. In some embodiments of any of the aspects, the vector and/or nucleic acid sequence described herein is codon-optimized for expression in a bacterial cell. In some embodiments of any of the aspects, the vector and/or nucleic acid sequence described herein is codon-optimized for expression in an E. coli cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding a nucleic acid or polypeptide as described herein in place of non-essential viral genes. The vector and/or particle may be utilized for the purpose of transferring any nucleic acids into cells either in vitro or in vivo. Numerous forms of viral vectors are known in the art.

It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean+1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third non-target entity. A reagent specific for a given target is one that exhibits specific binding for that target under the conditions of the assay being utilized.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in nucleic acid or protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A set of at least two readout molecules, each readout molecule comprising:
   a. a 3′ barcode-hybridizing region of nucleotides or analogs thereof, the region comprising a unique sequence distinct from the 3′ region sequence of all other readout molecules in the set;
   b. a 5′ non-barcode-hybridizing region of nucleotides or analogs thereof, the region comprising a sequence identical to the 5′ region sequence of all other readout molecules in the set;
   c. a sulfur modification in place of the bridged oxygen of the phosphate backbone between the 5′ and 3′ regions; and
   d. an optically detectable label.
2. The set of paragraph 1, wherein the label is a fluorescent label.
3. The set of any one of paragraphs 1-2, wherein the optically-detectable label comprises or further comprises biotin, amines, metals, metal nanoclusters, anchoring molecules, quantum dotes, or acrydite.
4. The set of any one of paragraphs 1-3, wherein the label is located at the 5′ end of the readout molecule.
5. The set of any one of paragraphs 1-4, wherein the set comprises four distinguishable labels.
6. The set of any one of paragraphs 1-5, wherein the set comprises at least two distinguishable labels.
7. The set of any one of paragraphs 1-6, wherein the set comprises at least three distinguishable labels.
8. The set of any one of paragraphs 1-7, wherein the set comprises at least four distinguishable labels.
9. The set of any one of paragraphs 1-8, wherein the readout molecules of each set which comprise a first 3′ region only comprise a first distinguishable label.
10. The set of any one of paragraphs 1-9, wherein the readout molecules of each set which comprise any selected 3′ region only comprise a corresponding given distinguishable label.
11. The set of any one of paragraphs 1-10, wherein the 3′ region is at least 1 nucleotide or analog thereof in length.
12. The set of any one of paragraphs 1-11, wherein the 3′ region is 5 nucleotides or analogs thereof in length.
13. The set of any one of paragraphs 1-12, wherein the 5′ region comprises only universal nucleotide bases.
14. The set of any one of paragraphs 1-13, wherein the 5′ region comprises only deoxyinosine nucleotides.
15. The set of any one of paragraphs 1-14, wherein the 5′ region is at least 1 nucleotide or analog thereof in length.
16. The set of any one of paragraphs 1-15, wherein the 5′ region is 3 nucleotides or analogs thereof in length.
17. The set of any one of paragraphs 1-16, wherein the at least two readout molecule are DNA or RNA.
18. A method of detecting at least one target molecule in a sample, the method comprising:
   a. contacting the sample with at least one oligonucleotide tag, each oligonucleotide tag comprising:
      i. a recognition domain that binds specifically to a target molecule to be detected, and
      ii. a street comprising a barcode region that comprises at least one barcode bit;
   b. contacting the sample with a set of readout molecules according to any one of paragraphs 1-17; and
   c. detecting the relative order of the optically detectable labels hybridized to the at least one oligonucleotide tag, wherein the at least one oligonucleotide tag is hybridized to the at least one target molecule, whereby the relative order of the optically detectable labels permits identification of which oligonucleotide tag is hybridized to the target molecule at that location.
19. The method of paragraph 18, wherein the barcode region is unique to each oligonucleotide tag.
20. The method of any one of paragraphs 18-19, wherein the total number of unique barcode bits is less than the total number of unique barcode bits possible.
21. The method of any one of paragraphs 18-20, wherein the total number of unique barcode bits is less than 10% of the total number of unique barcode bits possible.
22. The method of any one of paragraphs 18-21, wherein the total number of unique barcode bits is less than 1% of the total number of unique barcode bits possible.
23. The method of any one of paragraphs 18-229, wherein the total number of unique barcode bits is at least 2 unique barcode bits.
24. The method of any one of paragraphs 18-23, wherein the total number of unique barcode bits is no more than 10 unique barcode bits.
25. The method of any one of paragraphs 18-24, wherein the barcode-hybridizing region is unique to each readout molecule.
26. The method of any one of paragraphs 18-25, wherein the total number of unique barcode-hybridizing regions used in the set of readout molecules is less than the total number of unique barcode-hybridizing regions possible.
27. The method of any one of paragraphs 18-26, wherein the total number of unique barcode-hybridizing regions in the set of readout molecules is less than 10% of the total number of unique barcode-hybridizing regions possible.
28. The method of any one of paragraphs 18-27, wherein the total number of unique barcode-hybridizing regions in the set of readout molecules is less than 1% of the total number of unique barcode-hybridizing regions possible.
29. The method of any one of paragraphs 18-28, wherein the total number of unique barcode-hybridizing regions in the set of readout molecules comprises at least 2 unique barcode-hybridizing regions.
30. The method of any one of paragraphs 18-29, wherein the total number of unique barcode-hybridizing regions in the set of readout molecules comprises no more than 10 unique barcode-hybridizing regions.
31. The method of any one of paragraphs 18-30, wherein the street further comprises a primer binding region for annealing a sequencing primer.
32. The method of any one of paragraphs 18-31, wherein the detecting step is performed with a sequencing method.
33. The method of any one of paragraphs 18-32, wherein the sequencing method comprises sequencing by ligation, sequencing by synthesis, sequencing by hybridization, and/or sequencing by cyclic reversible polymerization hybridization chain reaction.
34. The method of any one of paragraphs 18-33, wherein sequencing by ligation comprises enzyme-based ligation.
35. The method of any one of paragraphs 18-34, wherein sequencing by ligation comprises chemical ligation, copper assisted ligation, copper free click reaction, Amine-EDC based coupling, or thiol-maleimide Michael addition.
36. The method of any one of paragraphs 18-35, wherein the specific hybridization of a readout molecule to a street is determined by the identity of the barcode region and barcode-hybridizing region.
37. The method of any one of paragraphs 18-36, wherein the optically-detectable label is a fluorophore.
38. The method of any one of paragraphs 18-37, wherein the detecting is performed with fluorescence microscopy.
39. The method of any one of paragraphs 18-38, wherein the optically-detectable label further comprises biotin, amines, metals, metal nanoclusters, anchoring molecules, quantum dotes, or acrydite.
40. The method of any one of paragraphs 18-39, wherein the detecting is performed with at least single cell resolution.
41. The method of any one of paragraphs 18-40, wherein at least 2 target molecules are detected concurrently.
42. The method of any one of paragraphs 18-41, wherein at least 3 target molecules are detected concurrently.
43. The method of any one of paragraphs 18-42, wherein at least 10 target molecules are detected concurrently.
44. The method of any one of paragraphs 18-43, wherein at least 20 target molecules are detected concurrently.
45. The method of any one of paragraphs 18-44, wherein the target molecule is a nucleic acid, a polypeptide, a cell surface molecule, or an inorganic material.
46. The method of any one of paragraphs 18-45, wherein the target molecule is DNA or RNA.
47. The method of any one of paragraphs 18-46, wherein the target molecule is linked to a nucleic acid, a polypeptide, a cell surface molecule, or an inorganic material.
48. The method of any one of paragraphs 18-47, wherein the sample is a cell, cell culture, or tissue sample.

Some embodiments of the technology described herein can be defined according to any of the following numbered paragraphs:
1. A set of at least two readout molecules, each readout molecule comprising:
   a. a 3′ barcode-hybridizing region of nucleotides or analogs thereof, the region comprising a unique sequence distinct from the 3′ region sequence of all other readout molecules in the set;
   b. a 5′ non-barcode-hybridizing region of nucleotides or analogs thereof, the region comprising a sequence identical to the 5′ region sequence of all other readout molecules in the set;
   c. a sulfur modification in place of the bridged oxygen of the phosphate backbone between the 5′ and 3′ regions; and
   d. an optically detectable label.
2. The set of paragraph 1, wherein the label is a fluorescent label.
3. The set of any one of paragraphs 1-2, wherein the optically-detectable label comprises or further comprises biotin, amines, metals, metal nanoclusters, noble metal nanoparticles, anchoring molecules, quantum dots, acrydite, or DNA origami structures.
4. The set of any one of paragraphs 1-3, wherein the label is located at the 5′ end of the readout molecule.
5. The set of any one of paragraphs 1-4, wherein the set comprises four distinguishable labels.
6. The set of any one of paragraphs 1-5, wherein the set comprises at least two distinguishable labels.
7. The set of any one of paragraphs 1-6, wherein the set comprises at least three distinguishable labels.
8. The set of any one of paragraphs 1-7, wherein the set comprises at least four distinguishable labels.
9. The set of any one of paragraphs 1-8, wherein the readout molecules of each set which comprise a first 3′ region only comprise a first distinguishable label.
10. The set of any one of paragraphs 1-9, wherein the readout molecules of each set which comprise any selected 3′ region only comprise a corresponding given distinguishable label.
11. The set of any one of paragraphs 1-10, wherein the 3′ region is at least 1 nucleotide or analog thereof in length.
12. The set of any one of paragraphs 1-11, wherein the 3′ region is 5 nucleotides or analogs thereof in length.
13. The set of any one of paragraphs 1-12, wherein the 5′ region comprises only universal nucleotide bases.
14. The set of any one of paragraphs 1-13, wherein the 5′ region comprises only deoxyinosine nucleotides.
15. The set of any one of paragraphs 1-14, wherein the 5′ region is at least 1 nucleotide or analog thereof in length.
16. The set of any one of paragraphs 1-15, wherein the 5′ region is 3 nucleotides or analogs thereof in length.
17. The set of any one of paragraphs 1-16, wherein the at least two readout molecules comprise DNA and/or RNA.
18. The set of any one of paragraphs 1-17, wherein the at least two readout molecules consist of or consist essentially of DNA and/or RNA.
19. The set of any one of paragraphs 1-18, wherein the at least two readout molecules comprise a polypeptide.
20. A set of at least two readout molecules, each readout molecule comprising:
   a. a 3′ barcode-hybridizing region of nucleotides or analogs thereof, the region comprising a unique sequence distinct from the 3′ region sequence of all other readout molecules in the set;
   b. a 5′ non-barcode-hybridizing region of nucleotides or analogs thereof, and
   c. a sulfur modification in place of the bridged oxygen of the phosphate backbone between the 5′ and 3′ regions.
21. The set of paragraph 20, wherein the 5′ non-barcode-hybridizing region of at least one readout molecule specifically hybridizes to an oligonucleotide.
22. The set of any one of paragraphs 20-21, wherein the oligonucleotide comprises at least one detectable label.
23. The set of any one of paragraphs 20-22, wherein the oligonucleotide specifically hybridizes to at least one other oligonucleotide.
24. The set of any one of paragraphs 20-23, wherein the oligonucleotide is an amplification primer.
25. The set of any one of paragraphs 20-24, wherein the oligonucleotide is a sequencing primer.
26. The set of any one of paragraphs 20-25, wherein the oligonucleotide is an imager strand for super resolution microscopy.
27. The set of any one of paragraphs 20-26, wherein the 5′ non-barcode-hybridizing region of at least one readout molecule is at least 5 nucleotides long.
28. The set of any one of paragraphs 20-27, wherein the 5′ non-barcode-hybridizing region of at least one readout molecule is at least 10 nucleotides long.
29. The set of any one of paragraphs 20-28, wherein the 5′ non-barcode-hybridizing region comprises a sequence identical to the 5′ region sequence of all other readout molecules in the set.
30. The set of any one of paragraphs 20-29, wherein at least one readout molecule comprises an optically detectable label.
31. The set of any one of paragraphs 20-30, wherein the label of at least one readout molecule is a fluorescent label.
32. The set of any one of paragraphs 20-31, wherein the optically-detectable label comprises or further comprises a fluorophore, biotin, amines, metals, metal nanoclusters, noble metal nanoparticles, anchoring molecules, quantum dots, acrydite, or DNA origami structures.
33. A readout molecule comprising:
   a. a 3′ barcode-hybridizing region of nucleotides or analogs thereof, the region comprising a unique sequence distinct from the 3′ region sequence of all other readout molecules in a set of readout molecules;
   b. a 5′ non-barcode-hybridizing region of nucleotides or analogs thereof, the region comprising a sequence identical to the 5′ region sequence of all other readout molecules in the set;
   c. a sulfur modification in place of the bridged oxygen of the phosphate backbone between the 5′ and 3′ regions;
   d. an optically detectable label; and
   e. a nanoparticle.
34. A readout molecule comprising:
   a. a 3′ barcode-hybridizing region of nucleotides or analogs thereof, the region comprising a unique sequence distinct from the 3′ region sequence of all other readout molecules in a set of readout molecules;
   b. a 5′ non-barcode-hybridizing region of nucleotides or analogs thereof, the region comprising a sequence identical to the 5′ region sequence of all other readout molecules in the set;
   c. a sulfur modification in place of the bridged oxygen of the phosphate backbone between the 5′ and 3′ regions; and
   d. a metal nanoparticle.
35. A readout molecule comprising:
   a. a 3′ barcode-hybridizing region of nucleotides or analogs thereof,
   b. a 5′ non-barcode-hybridizing region of nucleotides or analogs thereof,
   c. a sulfur modification in place of the bridged oxygen of the phosphate backbone between the 5′ and 3′ regions; and
   d. a metal nanoparticle.
36. The readout molecule of any one of paragraphs 34 or 35, further comprising an optically detectable label.
37. A set of at least two readout molecules, each readout molecule comprising:
   a. a 3′ barcode-hybridizing region of nucleotides or analogs thereof, the region comprising a unique sequence distinct from the 3′ region sequence of all other readout molecules in the set;
   b. a 5′ non-barcode-hybridizing region of nucleotides or analogs thereof, the region comprising a sequence identical to the 5′ region sequence of all other readout molecules in the set;
   c. a sulfur modification in place of the bridged oxygen of the phosphate backbone between the 5′ and 3′ regions; and
   d. an optically detectable label;
wherein at least one readout molecule further comprises a nanoparticle.
38. A set of at least two readout molecules, each readout molecule comprising:
   a. a 3′ barcode-hybridizing region of nucleotides or analogs thereof, the region comprising a unique sequence distinct from the 3′ region sequence of all other readout molecules in the set;
   b. a 5′ non-barcode-hybridizing region of nucleotides or analogs thereof, the region comprising a sequence identical to the 5′ region sequence of all other readout molecules in the set; and
   c. a sulfur modification in place of the bridged oxygen of the phosphate backbone between the 5′ and 3′ regions;
   wherein at least one readout molecule further comprises a nanoparticle.
39. The set of paragraph 38, wherein at least one readout molecule further comprises an optically detectable label.
40. The readout molecule or set thereof of any one of paragraphs 33-39, wherein the optically detectable label comprises a fluorophore.
41. The readout molecule or set thereof of any one of paragraphs 33-40, wherein the nanoparticle is linked to at least two readout molecules of the set.
42. The readout molecule or set thereof of any one of paragraphs 33-41, wherein the nanoparticle comprises a metal nanoparticle.
43. The readout molecule or set thereof of any one of paragraphs 33-42, wherein the metal nanoparticle is selected from the group consisting of Au, Ag, Ni, Co, Pt, Pd, Cu, Ti, and Al nanoparticles.
44. The readout molecule or set thereof of any one of paragraphs 33-43, wherein the nanoparticle comprises a gold nanoparticle.
45. The readout molecule or set thereof of any one of paragraphs 33-44, wherein the nanoparticle comprises a gold nanorod.
46. The readout molecule or set thereof of any one of paragraphs 33-45, wherein the nanoparticle has a diameter of about 1.2 nm.
47. The readout molecule or set thereof of any one of paragraphs 33-46, wherein the nanoparticle has a diameter of about 3 nm.
48. The readout molecule or set thereof of any one of paragraphs 33-47, wherein the nanoparticle has a diameter of about 5 nm.
49. The readout molecule or set thereof of any one of paragraphs 33-48, wherein the nanoparticle has a diameter of about 10 nm.
50. The readout molecule or set thereof of any one of paragraphs 33-49, wherein the nanoparticle has a diameter of about 30 nm.
51. The readout molecule or set thereof of any one of paragraphs 33-50, wherein the nanoparticle has a diameter of about 50 nm.
52. The readout molecule or set thereof of any one of paragraphs 33-51, wherein the nanoparticle is at the 3′ end of the readout molecule.
53. The readout molecule or set thereof of any one of paragraphs 33-52, wherein the nanoparticle is at least 20 nucleotides from the detectable label.
54. The readout molecule or set thereof of any one of paragraphs 33-53, wherein the nanoparticle is at least 30 nucleotides from the detectable label.
55. Use of the readout molecule or set thereof of any one of paragraphs 1-54, for:
   a. detection of at least one target molecule;
   b. signal amplification;
   c. branch reactions;
   d. hybridization chain reaction (HCR);
   e. signal amplification by exchange reaction (SABER);
   f. rolling circle amplification (RCA);
   g. in situ sequencing;
   h. matrix attachment; or
   i. super resolution microscopy.
56. A method of detecting at least one target molecule in a sample, the method comprising:
   a. contacting the sample with at least one oligonucleotide tag, each oligonucleotide tag comprising:
      i. a recognition domain that binds specifically to a target molecule to be detected, and
      ii. a street comprising a barcode region that comprises at least one barcode bit;
   b. contacting the sample with a set of readout molecules according to any one of paragraphs 1-54; and
   c. detecting the relative order of the optically detectable labels hybridized to the at least one oligonucleotide tag, wherein the at least one oligonucleotide tag is hybridized to the at least one target molecule, whereby the relative order of the optically detectable labels permits identification of which oligonucleotide tag is hybridized to the target molecule at that location.
57. The method of paragraph 56, wherein the barcode region is unique to each oligonucleotide tag.
58. The method of any one of paragraphs 56-57, wherein the total number of unique barcode bits is less than the total number of unique barcode bits possible.
59. The method of any one of paragraphs 56-58, wherein the total number of unique barcode bits is less than 10% of the total number of unique barcode bits possible.
60. The method of any one of paragraphs 56-59, wherein the total number of unique barcode bits is less than 1% of the total number of unique barcode bits possible.
61. The method of any one of paragraphs 56-60, wherein the total number of unique barcode bits is at least 2 unique barcode bits.
62. The method of any one of paragraphs 56-61, wherein the total number of unique barcode bits is no more than 10 unique barcode bits.
63. The method of any one of paragraphs 56-62, wherein the barcode-hybridizing region is unique to each readout molecule.
64. The method of any one of paragraphs 56-63, wherein the total number of unique barcode-hybridizing regions used in the set of readout molecules is less than the total number of unique barcode-hybridizing regions possible.
65. The method of any one of paragraphs 56-64, wherein the total number of unique barcode-hybridizing regions in the set of readout molecules is less than 10% of the total number of unique barcode-hybridizing regions possible.
66. The method of any one of paragraphs 56-65, wherein the total number of unique barcode-hybridizing regions in the set of readout molecules is less than 1% of the total number of unique barcode-hybridizing regions possible.
67. The method of any one of paragraphs 56-66, wherein the total number of unique barcode-hybridizing regions in the set of readout molecules comprises at least 2 unique barcode-hybridizing regions.
68. The method of any one of paragraphs 56-67, wherein the total number of unique barcode-hybridizing regions in the set of readout molecules comprises no more than 10 unique barcode-hybridizing regions.
69. The method of any one of paragraphs 56-68, wherein the street further comprises a primer binding region for annealing a sequencing primer.
70. The method of any one of paragraphs 56-69, wherein the detecting step is performed with a sequencing method.
71. The method of any one of paragraphs 56-70, wherein the sequencing method comprises sequencing by ligation, sequencing by synthesis, sequencing by hybridization, and/or sequencing by cyclic reversible polymerization hybridization chain reaction.
72. The method of any one of paragraphs 56-71, wherein sequencing by ligation comprises enzyme-based ligation.
73. The method of any one of paragraphs 56-72, wherein sequencing by ligation comprises chemical ligation, copper assisted ligation, copper free click reaction, Amine-EDC based coupling, or thiol-maleimide Michael addition.
74. The method of any one of paragraphs 56-73, wherein the specific hybridization of a readout molecule to a street is determined by the identity of the barcode region and barcode-hybridizing region.
75. The method of any one of paragraphs 56-74, wherein the optically-detectable label is a fluorophore.
76. The method of any one of paragraphs 56-75, wherein the detecting is performed with fluorescence microscopy.
77. The method of any one of paragraphs 56-76, wherein the optically-detectable label further comprises biotin, amines, metals, metal nanoclusters, noble metal nanoparticles, anchoring molecules, quantum dots, acrydite, or DNA origami structures.
78. The method of any one of paragraphs 56-77, wherein the detecting is performed with at least single cell resolution.
79. The method of any one of paragraphs 56-78, wherein the detecting is performed with at least single nucleus resolution.
80. The method of any one of paragraphs 56-79, wherein at least 2 target molecules are detected concurrently.
81. The method of any one of paragraphs 56-80, wherein at least 3 target molecules are detected concurrently.
82. The method of any one of paragraphs 56-81, wherein at least 10 target molecules are detected concurrently.
83. The method of any one of paragraphs 56-82, wherein at least 20 target molecules are detected concurrently.
84. The method of any one of paragraphs 56-83, wherein the target molecule comprises a nucleic acid, a polypeptide, a cell surface molecule, or an inorganic material.
85. The method of any one of paragraphs 56-84, wherein the target molecule comprises DNA and/or RNA.
86. The method of any one of paragraphs 56-85, wherein the target molecule comprises a polypeptide.
87. The method of any one of paragraphs 56-86, wherein the target molecule is covalently or non-covalently linked to a nucleic acid, a polypeptide, a cell surface molecule, or an inorganic material.
88. The method of any one of paragraphs 56-87, wherein the sample is a cell, cell culture, or tissue sample.
89. The method of any one of paragraphs 56-88, wherein the sample comprises organoids.
90. An enhanced method of detecting at least one target molecule in a sample, the method comprising:
   a. contacting the sample with at least one oligonucleotide tag, each oligonucleotide tag comprising:
      i. a recognition domain that binds specifically to a target molecule to be detected, and
      ii. a street comprising a barcode region that comprises at least one barcode bit;
   b. contacting the sample with a readout molecule or set thereof according to any one of paragraphs 1-54; and
   c. detecting the relative order of the optically detectable labels hybridized to the at least one oligonucleotide tag, wherein the at least one oligonucleotide tag is hybridized to the at least one target molecule, whereby the relative order of the optically detectable labels permits identification of which oligonucleotide tag is hybridized to the target molecule at that location.
91. The method of paragraph 90, wherein the signal of the optically detectable label of the at least one readout molecule comprising a nanoparticle is increased at least 1.5-fold compared to the signal of the optically detectable label of the same readout molecule not comprising the nanoparticle.
92. The method of any one of paragraphs 90-91, wherein the signal of the optically detectable label of the at least one readout molecule comprising a nanoparticle is increased at least 3-fold compared to the signal of the optically detectable label of the same readout molecule not comprising the nanoparticle.
93. The method of any one of paragraphs 90-92, wherein the signal of the optically detectable label of the at least one readout molecule comprising a nanoparticle is increased at least 10-fold compared to the signal of the optically detectable label of the same readout molecule not comprising the nanoparticle.
94. The method of any one of paragraphs 90-93, wherein the signal of the optically detectable label of the at least one readout molecule comprising a nanoparticle is increased at least 50-fold compared to the signal of the optically detectable label of the same readout molecule not comprising the nanoparticle.
95. The method of any one of paragraphs 90-94, wherein the sample comprises a human cell nucleus.
96. The method of any one of paragraphs 90-95, wherein the sample comprises a nucleus from the cell of any organism.
97. The method of any one of paragraphs 90-96, wherein the sample comprises metaphase chromosome spreads.
98. The method of any one of paragraphs 90-97, wherein the metaphase chromosomes are obtained from a cultured cell nucleus.
99. The method of any one of paragraphs 90-98, wherein the metaphase chromosomes are obtained from a nucleus extracted from a tissue section, an organoid, or a biopsy specimen.
100. The method of any one of paragraphs 90-99, wherein the detectable labels are detected using electron microscopy, fluorescence microscopy, dark field microscopy, or any combination thereof.
101. A method of karyotyping a biological sample, the method comprising:
   a. contacting the sample with at least one oligonucleotide tag specific to at least one chromosome, each oligonucleotide tag comprising:
      i. a recognition domain that binds specifically to a target molecule to be detected, and
      ii. a street comprising a barcode region that comprises at least one barcode bit;
   b. contacting the sample with a set of readout molecules according to any one of paragraphs 1-54;
   c. detecting the relative order of the optically detectable labels hybridized to the at least one oligonucleotide tag, wherein the at least one oligonucleotide tag is hybridized to the at least one target molecule, whereby the relative order of the optically detectable labels permits identification of which oligonucleotide tag is hybridized to the target molecule at that location; and
   d. determining the identity of at least one chromosome according to the identity of the least one oligonucleotide tag specific to the at least one chromosome.
102. The method of paragraph 101, wherein the sample is contacted with at least one oligonucleotide tag specific to the p arm of the at least one chromosome.
103. The method of any one of paragraphs 101-102, wherein the sample is contacted with at least one oligonucleotide tag specific to the q arm of the at least one chromosome.
104. The method of any one of paragraphs 101-103, wherein the sample is contacted with at least one oligonucleotide tag specific to the p arm of the at least one chromosome, and at least one oligonucleotide tag specific to the q arm of the at least one chromosome.
105. The method of any one of paragraphs 101-104, wherein the sample is contacted with at least two oligonucleotide tags specific to the p arm or the q arm of the at least one chromosome.
106. The method of any one of paragraphs 101-105, wherein the sample is contacted with at least three oligonucleotide tags specific to the p arm or the q arm of the at least one chromosome.
107. The method of any one of paragraphs 101-106, wherein the sample is contacted with at most 6 oligonucleotide tags specific to each chromosome arm.
108. The method of any one of paragraphs 101-107, wherein the sample is contacted with at most 10 oligonucleotide tags specific to each chromosome arm.
109. The method of any one of paragraphs 101-108, wherein the sample is contacted with at most 20 oligonucleotide tags specific to each chromosome arm.
110. The method of any one of paragraphs 101-109, wherein the sample comprises a human cell nucleus.
111. The method of any one of paragraphs 101-110, wherein the sample comprises a nucleus from the cell of any organism.
112. The method of any one of paragraphs 101-111, wherein the sample comprises metaphase chromosome spreads.
113. The method of any one of paragraphs 101-112, wherein the metaphase chromosomes are obtained from a cultured cell nucleus.
114. The method of any one of paragraphs 101-113, wherein the metaphase chromosomes are obtained from a nucleus extracted from a tissue section, an organoid, or a biopsy specimen.
115. A method of producing a high resolution image of at least one target molecule in a sample, the method comprising:
   a. imaging the at least one target molecule using at least one round of a high resolution imaging method; and
   b. determining the identity of the at least one imaged target molecule, comprising:
      i. contacting the sample with at least one oligonucleotide tag, each oligonucleotide tag comprising:
         A. a recognition domain that binds specifically to a target molecule to be detected, and
         B. a street comprising a barcode region that comprises at least one barcode bit;
      ii. contacting the sample with a set of readout molecules according to any one of paragraphs 1-54; and
      iii. detecting the relative order of the optically detectable labels hybridized to the at least one oligonucleotide tag, wherein the at least one oligonucleotide tag is hybridized to the at least one target molecule, whereby the relative order of the optically detectable labels permits identification of which oligonucleotide tag is hybridized to the target molecule at that location.
116. The method of paragraph 115, wherein the method comprises imaging at least 2 target molecules.
117. The method of any one of paragraphs 115-116, wherein the method comprises imaging at least 12 target molecules.
118. The method of any one of paragraphs 115-117, wherein the method comprises imaging at least 66 target molecules.
119. The method of any one of paragraphs 115-118, wherein the method comprises imaging at least 258 target molecules.
120. The method of any one of paragraphs 115-119, wherein the method comprises imaging at least 500 target molecules.
121. The method of any one of paragraphs 115-120, wherein the method comprises imaging at least 5000 target molecules.
122. The method of any one of paragraphs 115-121, wherein all of the target molecules are imaged at one time.
123. The method of any one of paragraphs 115-122, wherein at least half of the target molecules are imaged at one time.
124. The method of any one of paragraphs 115-123, wherein the method comprises at least two rounds of the high resolution imaging method.
125. The method of any one of paragraphs 115-124, wherein the method comprises at least three rounds of the high resolution imaging method.
126. The method of any one of paragraphs 115-125, wherein the method comprises at least five rounds of the high resolution imaging method.
127. The method of any one of paragraphs 115-126, wherein the method comprises at least 20 rounds of the high resolution imaging method.
128. The method of any one of paragraphs 115-127, wherein the high resolution imaging method is selected from the group consisting of: Oligo Stochastic Optical Reconstruction Microscopy (OligoSTORM); structured illumination microscopy (SIM); Stimulated emission depletion (STED) microscopy; and Oligo DNA point accumulation in nanoscale topology (DNA-PAINT).
129. The method of any one of paragraphs 115-128, wherein the high resolution imaging method comprises Oligo Stochastic Optical Reconstruction Microscopy (OligoSTORM).
130. The method of any one of paragraphs 115-129, wherein detecting the relative order of the optically detectable labels hybridized to the at least one oligonucleotide tag comprises at least 2 rounds of contacting the sample with the set of readout molecules.
131. The method of any one of paragraphs 115-130, wherein detecting the relative order of the optically detectable labels hybridized to the at least one oligonucleotide tag comprises at least 3 rounds of contacting the sample with the set of readout molecules.
132. The method of any one of paragraphs 115-131, wherein detecting the relative order of the optically detectable labels hybridized to the at least one oligonucleotide tag comprises at least 5 rounds of contacting the sample with the set of readout molecules.
133. The method of any one of paragraphs 115-132, wherein detecting the relative order of the optically detectable labels hybridized to the at least one oligonucleotide tag comprises at least 10 rounds of contacting the sample with the set of readout molecules.
134. The method of any one of paragraphs 115-133, wherein detecting the relative order of the optically detectable labels hybridized to the at least one oligonucleotide tag comprises at least 20 rounds of contacting the sample with the set of readout molecules.
135. The method of any one of paragraphs 115-134, wherein the at least one target molecule comprises a 1 kb nucleic acid.
136. The method of any one of paragraphs 115-135, wherein the at least one target molecule comprises a 15 kb nucleic acid.
137. The method of any one of paragraphs 115-136, wherein the at least one target molecule comprises a 50 kb nucleic acid.
138. The method of any one of paragraphs 115-137, wherein the at least one target molecule comprises a 100 kb nucleic acid.
139. The method of any one of paragraphs 115-138, wherein the at least one target molecule comprises a 1 Mb nucleic acid.
140. The method of any one of paragraphs 115-139, wherein the at least one target molecule comprises a chromosome.
141. The method of any one of paragraphs 115-140, wherein the at least one target molecule comprises a genome.

### EXAMPLES

### Example 1

Towards 3D Human Genome Mapping in 8 Rounds of In Situ Sequencing

Replication, inheritance, and developmentally regulated gene expression are all genome-wide processes, occurring across all chromosomes simultaneously. Indeed, disruption of their genome-wide coordination can lead to genome damage, chromosome breakage and loss, aneuploidy, gross misexpression of genes, and disease. As such, there has been increasing demand for technologies that have the potential to query genomes in their entirety. Of these diverse methods, those providing information regarding the spatial organization of the genome have been especially exciting, as there is burgeoning evidence that the three-dimensional (3D) arrangement of chromosomes is strongly correlated with genome function and stability. Assays using proximity-based capture, such as Hi-C and other chromosome conformation capture technologies, as well as Genome Architecture Mapping (GAM) have been particularly powerful, as they report frequencies with which genomic regions interact and/or are found in the same subsection of the nucleus. These genome-wide methods have revealed the hierarchical manner by which whole genomes are organized, from cis interactions between enhancers and promoters to the intra- and inter-chromosomal compartmentalization of active and inactive chromatin.

Methods such as fluorescent in-situ hybridization (FISH) also champion the genome as a highly, internally organized entity. As did earlier ISH methods using radioactive and enzymatic markers, FISH permits sequence-specific visualization of the genome, one of the most impactful milestones being the discovery that individual chromosomes can form distinct territories. Such studies have led to a myriad of observations, including correlations between the radial positioning of genes and their state of activity, associations between the occurrence of translocations and the proximity of the participating chromosomes, and the dynamic genome reorganizations associated with DNA repair.

A full understanding of genome function requires tools that provide spatial information as well as accommodate whole genomes and, to that end, described herein is a suite of technologies that can accomplish this whole-genome imaging goal. In particular, described herein are studies combining Oligopaints with fluorescent in-situ sequencing (FISSEQ) to produce three FISSEQ methods, collectively named OligoFISSEQ (Oligopaint+FISSEQ), that achieve highly multiplexed genome visualization at single cell resolution using wide-field microscopy. These strategies entail the sequencing of barcodes embedded in the Oligopaint probes, wherein one of the three strategies uses sequencing by ligation (SBL), another uses sequencing by synthesis (SBS), and a third uses sequencing by hybridization (SBH). As shown herein, all three forms of OligoFISSEQ can be used to sequence barcoded Oligopaints. Focusing on OligoFISSEQ with SBL, 66 genomic loci in human diploid PGP1 skin fibroblasts (male, XY) were mapped using only 4 rounds of sequencing. Also described herein is a method to significantly improve barcode detection and, using it in conjunction with OligoFISSEQ, the human X chromosome can be traced by mapping 46 regions along its length. Finally, presented herein is a variety of exemplary applications of OligoFISSEQ, including in the context of immunofluorescence for protein and DNA mapping as well as combining OligoFISSEQ with OligoSTORM to demonstrate a method by which multiple genomic targets ranging in size from kilobases to megabases can be visualized simultaneously at super resolution.

### Principle and Validation

The strategy underlying all three of the OligoFISSEQ technologies described herein is the identification of imaged regions through the in situ reading of barcodes contained on Oligopaint oligos. With the long-term goal being visualization of entire genomes, these technologies called for scalable methods capable of revealing chromosome organization. As such, used herein are Oligopaints, which are bioinformatically designed and renewable single stranded oligonucleotides (oligos) that can be targeted to the genome in a sequence specific fashion (see e.g., Beliveau et al. 2012, supra; see e.g., FIG. 1A). Importantly, Oligopaint oligos contain nongenomic sequences, called “streets”, which permit a multitude of functionalities, including amenability to amplification, multiplexing, barcoding, and indirect visualization through hybridization of fluorophore-labeled (secondary) oligos (see e.g., Beliveau et al. 2015, supra). Encoding barcodes onto Oligopaints in conjunction with sequential rounds of hybridization and visualization, have permitted Oligopainting to circumvent the limitations imposed in some experimental contexts by requirements for fluorophores that are spectrally distinct; in general, conventional microscopes are often restricted to using only 3 to 4 fluorophores at one time, due to overlapping fluorophore excitation and emission spectra.

FISSEQ was originally developed for transcript analysis, with more recent iterations of it including RNA-FISSEQ, BaristaSeq, and Spatially-resolved Transcript Amplicon Readout Mapping (STARmap) (see e.g., Lee et al. 2014, Science 343 (6177): 1360-1363; Chen et al. 2018, Nucleic Acids Research 46 (4): e22-e22; Wang et al. 2018, Science 361 (6400): eaat5691; the content of each of each is incorporated by reference herein in its entirety). All these methods leverage the nucleotide level genomic resolution afforded by Next Generation Sequencing (NGS), including sequencing by ligation (SBL), and sequencing by synthesis (SBS), to provide 3D spatial maps of transcripts within the cell (see e.g., Shendure et al., Science 309 (5741): 1728-32; 2005; Guo et al., 2008, Proc Natl Acad Sci USA. 2008 Jul. 8, 105(27):9145-50; Lee et al. 2014, supra; Chen et al. 2018, supra; Wang et al. 2018, supra). Without wishing to be bound by theory, it was reasoned that FISSEQ can be used to read barcoded Oligopaints and thus achieve genome scale target visualization. Targeting hundreds to thousands of identically barcoded Oligopaints to a genomic target can obviate the need for amplification typically required by FISSEQ and, thus, enhance the capacity of FISSEQ to reveal the underlying chromosomal structure of the targeted region. Furthermore, as FISSEQ can be carried out with widefield microscopy, strategies based on FISSEQ can permit the analysis of hundreds to thousands of cells at a time.

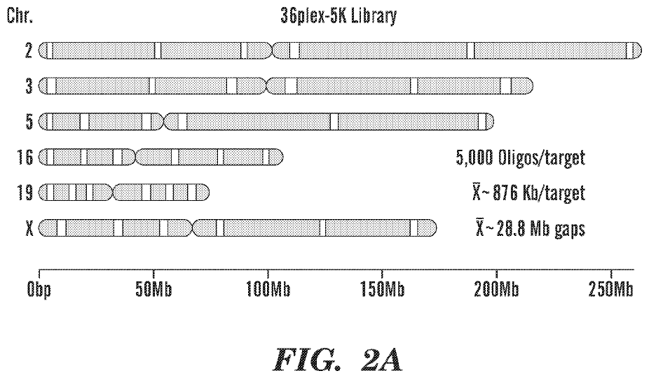

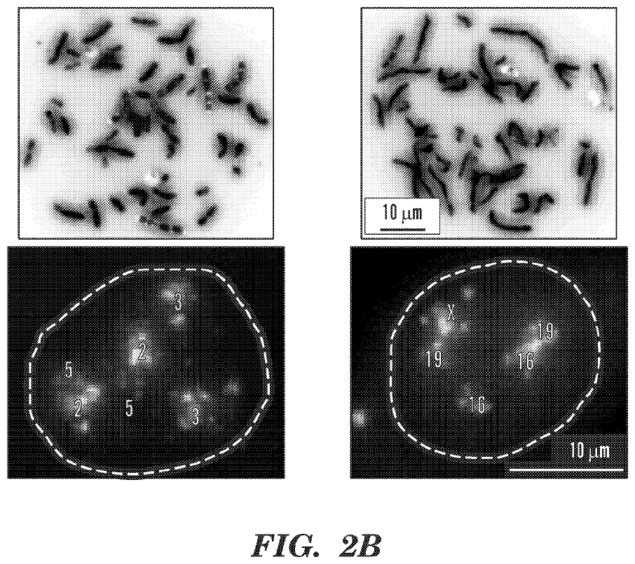

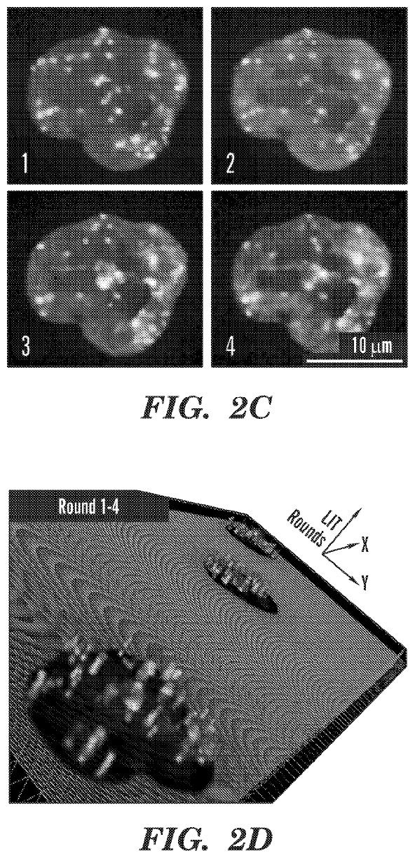

The ability to sequence the barcodes of Oligopaints that had been hybridized to the genome was assessed, focusing first on using SBL to effect ligation-based interrogation of genomic targets (LIT) and SBS to effect synthesis-based interrogation of targets (SIT) (see e.g., FIG. 2B). An Oligopaint library targeting 20,000 oligos to a 4.8 Mb single copy region on human chromosome 19 (Chr19-20K library) was used, due to its robust signal in conventional FISH (data not shown). Importantly, as Oligopaint streets can accommodate multiple barcodes, a single library was designed to accommodate both LIT and SIT, with the primer binding site and barcode for LIT embedded on Mainstreet (5′ end of Oligopaint) and the primer-binding site and barcode for SIT embedded on Backstreet (3′ end of Oligopaint) (see e.g., FIG. 1A). FIGS. 1B-1D outline OligoFISSEQ-LIT (0-LIT) and OligoFISSEQ-SIT (0-SIT) in more detail; LIT and SIT refer to the steps of sequencing, per se, while O-LIT and O-SIT refer to the use of LIT and SIT, respectively, in the context of OligoFISSEQ.

With O-LIT (see e.g., FIG. 1C), the barcode is read with SOLiD (Sequencing by Oligonucleotide Ligation and Detection) chemistry (see e.g., Valouev et al. 2008, Genome Res. 2008 Jul., 18(7): 1051-1063.; McKernan et al. 2009, Genome Res. 2009 Sep.;19(9):1527-41), wherein each bit of the LIT barcode (e.g., 5-nt per bit) is read by cleavable 8-mers containing one of four fluorophores. In brief, a sequencing primer is hybridized to the street and subsequent barcode readout begins by binding of the first barcode bit by a labeled 8-mer, which is then ligated and imaged. The 8-mer is then cleaved between nucleotides (nts) 5 and 6, leaving the first 5-nts and removing the label, allowing for the next LIT barcode bit to be read. In these initial studies, barcodes were used that, excluding the primer binding site, were 23 bases long and sufficient to accommodate 4 rounds of sequencing ([4 rounds of sequencing * 5-nts per bit]+the 3 nts uncleaved after the 4th round of sequencing); when fully utilized, four-bit or eight-bit barcodes can distinguish 256 or 65,536 targets, respectively. To minimize bias in barcode detection, automated thresholding algorithms were used that identified puncta in the first round, then measured signal intensities from these locations in the subsequent rounds, detecting the color with the highest signal over background ratio (SBR) (see e.g., METHODS); LIT, SIT, and HIT barcodes were all detected using slight variations of this method. Using O-LIT on the Chr19-20K library, 4-bit barcodes were successfully recovered from 92.1% 5.7% of cells (n=85 nuclei from 4 technical replicates; see e.g., FIG. 1F).

In the case of O-SIT, (see e.g., FIG. 1D), the barcode is sequenced using Illumina NextSeq™ chemistry, where each bit of the barcode (1-nt per bit) is read by reversible terminator nucleotides containing two fluorophores. In particular, deoxycytidine and deoxythymidine are labeled with fluorophores emitting in the red and green range of the spectrum, respectively, deoxyadenosine is labeled by a combination of these two fluorophores, and deoxyguanosine is unlabeled. Here, a sequencing primer is hybridized to the street, and subsequent reading of each bit (1-nt) of the barcode is accomplished by using the SIT barcode as a template to extend the primer one base at a time, with each round of extension followed by imaging and then removal of the label prior to the next round of extension. SIT barcodes are compact; excluding the primer binding sequence, the barcodes used herein were only 4 bases long and yet sufficient to accommodate 4 rounds of sequencing (4 rounds of sequencing * 1-nt per bit) and, if utilized fully, 256 targets; indeed, a SIT barcode only 8 bases would can target 65,536 targets. SIT was comparable to LIT as, using the Chr19-20K library, 4-bit barcodes were successfully recovered from 90.6%±5.8% of cells (n=66 cells from 4 replicates; see e.g., FIG. 1F).

In the process of encoding barcodes for SBL and SBS into Oligopaint streets, it was envisioned that the corresponding LIT and SIT barcodes could be repurposed as barcodes for a hybridization-based interrogation of targets (HIT) through sequencing by hybridization (SBH) (see e.g., FIG. 1A). In the case of SBH, targets are identified through the combinatoric use of multiple barcodes that are read via sequential hybridization of labeled secondary oligos. Such a strategy has enabled Oligopaints to be used for transcriptome profiling (see e.g., Chen et al. 2015, Science 348 (6233); Eng et al. 2017, Nature. 2019 April, 568(7751):235-239; Eng et al., Nature. 2019 April, 568(7751):235-239; the content of each of which is incorporated by reference herein in its entirety). Herein is described, for the first time, the use of SBH for in situ 3D spatial mapping of chromosomal DNA. In particular, OligoFISSEQ-HIT (0-HIT) was implemented by incorporating barcodes (20-nts per barcode) for SBH via two bridge oligos, with one bridge bound to the Mainstreet and spanning the junction between the primer binding site and the barcode used for O-LIT and the other bridge bound to Backstreet and spanning the junction between the same O-SIT components (see e.g., FIG. 1E). In particular, each of the two bridges included positions for two barcodes for a total of four positions, wherein each position encoded one of six possible barcodes that were then queried with six labeled secondary oligos; because three fluorophore species could be used, the secondary oligos were introduced in two rounds of hybridization, each round bringing in three differentially labeled secondary oligos for a total of eight rounds of hybridization to read all four HIT barcodes. This strategy can identify 1,296 targets (64), with the option to increase target capacity by adding additional choices for barcode sequences and/or extending the bridges to accommodate more barcode positions (data not shown). Note, while O-HIT can also be implemented using barcodes encoded directly into the streets, the barcodes were placed on bridges so as to permit the Oligopaint library to simultaneously accommodate SBL and SBS. Using O-HIT on the Chr19-20K library, 4-bit barcodes were successfully recovered from 91.6%±3.8% of cells (n=79 cells from 4 replicates (see e.g., FIG. 1F).

### OligoFISSEQ Via LIT

OligoFISSEQ was next used to address multiple loci on multiple chromosomes, and based on five arguments with respect to barcoding and scalability, efforts were focused on O-LIT; note that, given the continual emergence of innovations in sequencing and thus the impracticality of identifying any one strategy as superior, provided herein is proof-of-principle for the ability of OligoFISSEQ, by whatever sequencing strategy, to map whole genomes. Efforts were focused on LIT techniques because, first, LIT requires a positive signal for the reading of each bit. Without wishing to be bound by theory, it was reasoned that LIT would provide the most robust barcode. This was in contrast to SIT and HIT, for which at least some bits are encoded as absence of signal, thus rendering them vulnerable to the ambiguity of whether to interpret an absence of signal as informative (e.g., deoxyguanosine, in the case of SIT; absence of a specific barcode between rounds, in the case of HIT) or as the result of an inefficient round of chemistry or hybridization, respectively. Second, refraining from using absence of signal as informative can decrease the scalability of SIT and HIT significantly. For example, considering only four rounds of sequencing, reduction of the number of colors in the case of SIT from four to three would reduce the number of identifiable targets from 256 (4⁴) to 81 (3⁴). In the case of HIT, requiring all rounds of imaging to produce a signal would limit the number of possible barcode sequences for any barcode position to 3 such that the total number of targets that could be identified using four barcode positions would be 81 (34) rather than the 1,296 (64) described earlier. Third, in the case of SIT, wherein deoxyadenosine is detected by the presence of two fluorophores, one emitting in the red range of the spectrum and one emitting in the green, readout can become ambiguous should the ratios of signal over nuclear background (SBR) for the two fluorophores be too dissimilar. Fourth, all these aspects of HIT and SIT would become even more problematic in the later rounds of sequencing, when SBR decreases (see e.g., Methods). Finally, compared to LIT and SIT, HIT is less scalable. For example, whereas the current strategy for HIT has the capacity to localize 1,296 targets in eight rounds of hybridization, eight rounds of LIT and SIT could accommodate 65,536 targets; scaling HIT to accommodate this larger number of targets would require increasing barcode positions and a concomitant increase in the number of hybridizations from 8 to ˜14.

### Mapping 66 Genomic Targets

Next, an Oligopaint library (36plex-5K; see e.g., FIG. 2a) was designed targeting six regions along each of six chromosomes (2, 3, 5, 16, 19, and X) to assess the capacity of LIT to scale; 36plex-5K encompasses a total of 66 targets (6 targets for each of the 2 homologs of 5 autosomes in addition to 6 targets on the single X), each tiled by 5,000 (5K) Oligopaint oligos and, together, encompassing 31.6 Mb of the genome, with the genomic sizes of the individual targets ranging between 642 kb and 1.22 Mb (876 kb average). So that the path of the imaged chromosomes could be roughly trace, three targets were strategically positioned along each chromosome arm with one as close as possible to the telomere, the center of the arm, and to the centromere. Sites were chosen that were both gene poor (e.g., 5.4-6.1 genes/Mb; Chr 2, 3, 5, X) and gene rich (e.g., 10.8 and 23 genes/Mb; Chr 16 and 19, respectively), as well as large chromosomes (e.g., Chr 2: ˜247 Mb) and small chromosomes (e.g., Chr 19: ˜60 Mb). Depending on the chromosome arm, the distance between targets ranged from 7 Mb to 74.9 Mb, averaging 32.5 Mb. The number of Oligopaint oligos per target (5,000) was kept constant in order to assess the robustness of LIT to sequence targets of various length scales and densities (4-7.7 oligo targets/kb with an average 5.8). To assess the recovery of the same barcode at different genomic loci, two targets (Chr3qR3 and Chr5pR3) were encoded with identical barcodes. To assess overall specificity of 36plex-5K, the library was hybridized to metaphase chromosomes and interphase nuclei, visualizing targets with secondary oligos targeting chromosome-specific barcodes shared on the Backstreets of all oligos targeting the same chromosome. In metaphase chromosomes, the expected six-banded chromosomes were observed, and in interphase nuclei, the expected number of chromosome territories were observed (see e.g., FIG. 2B).

### An Every-Pixel Automated Analysis Pipeline

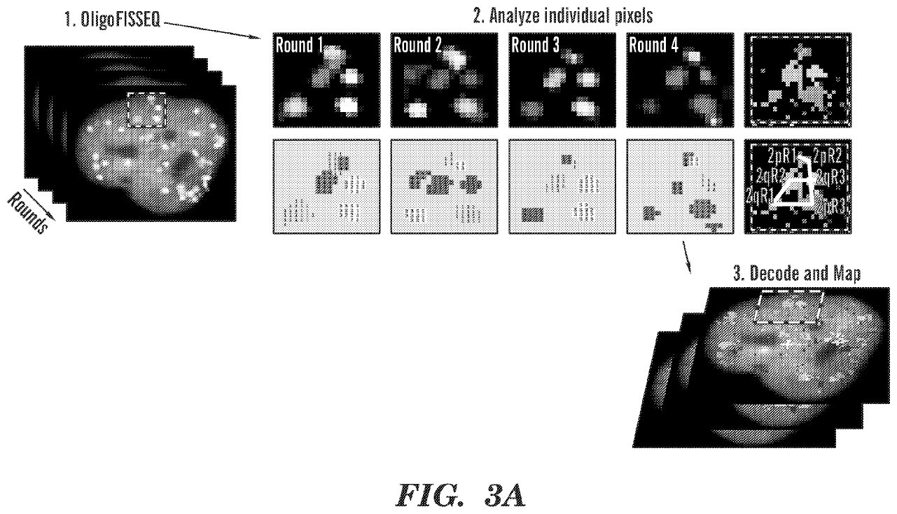

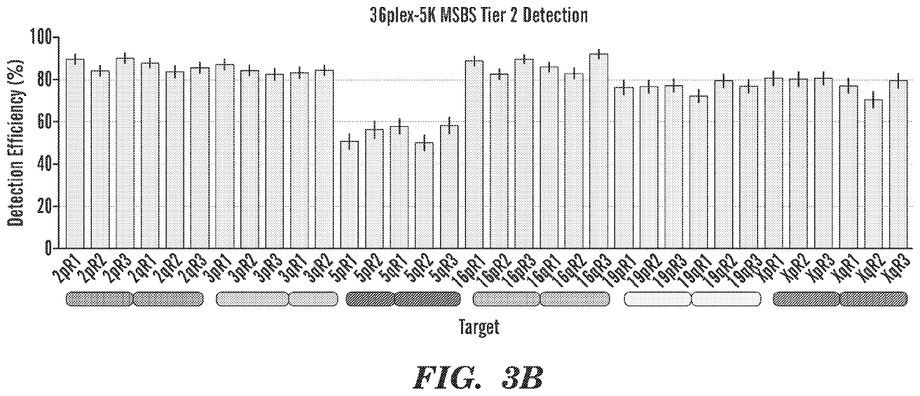

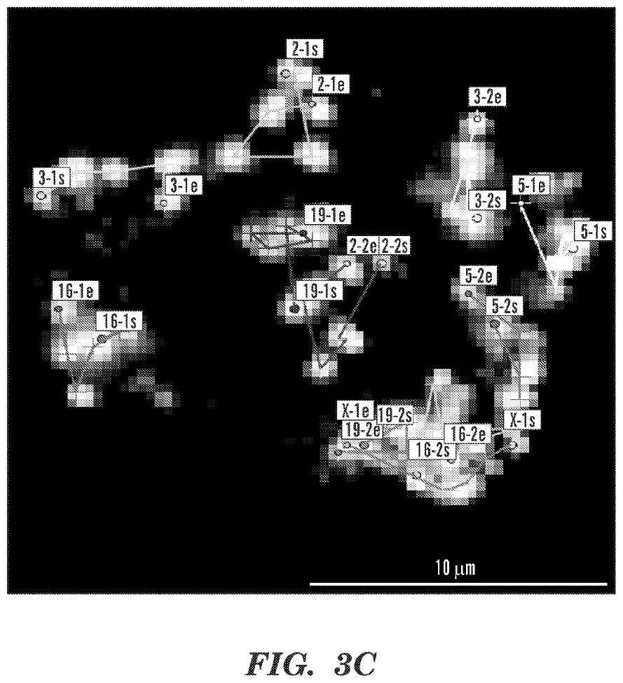

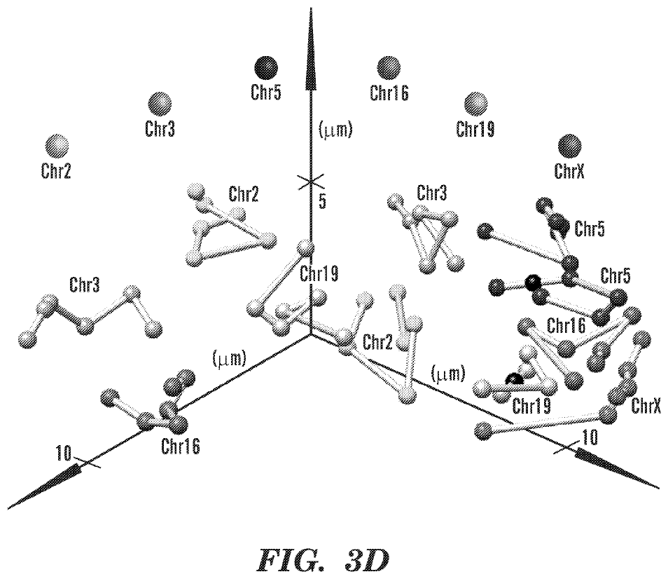

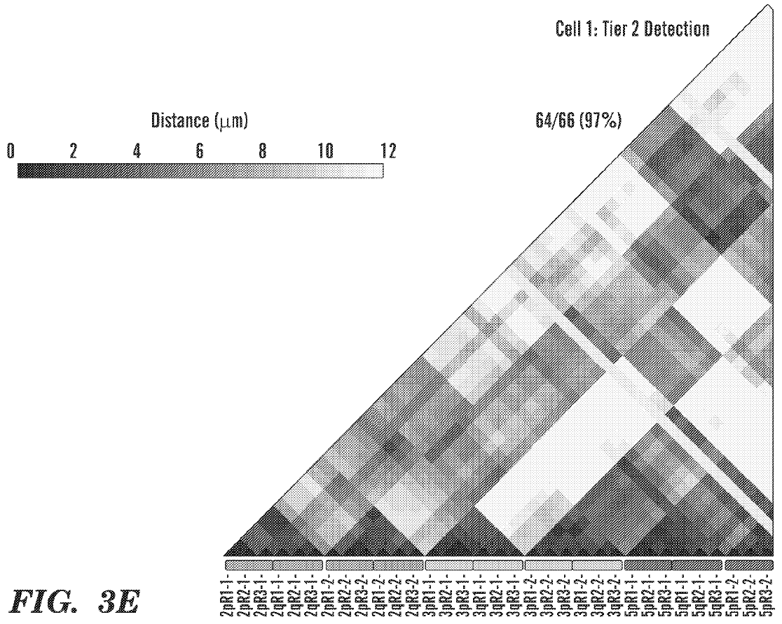

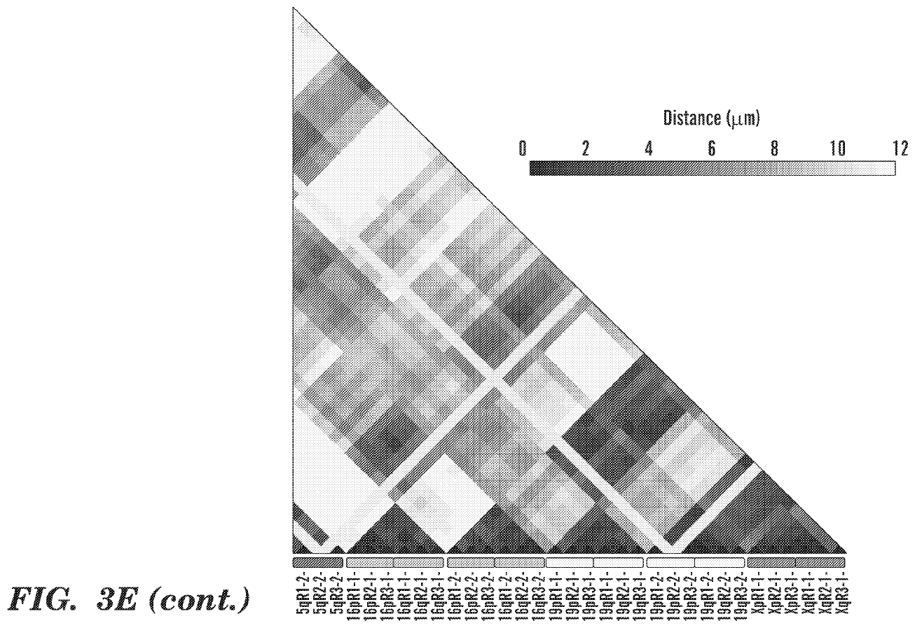

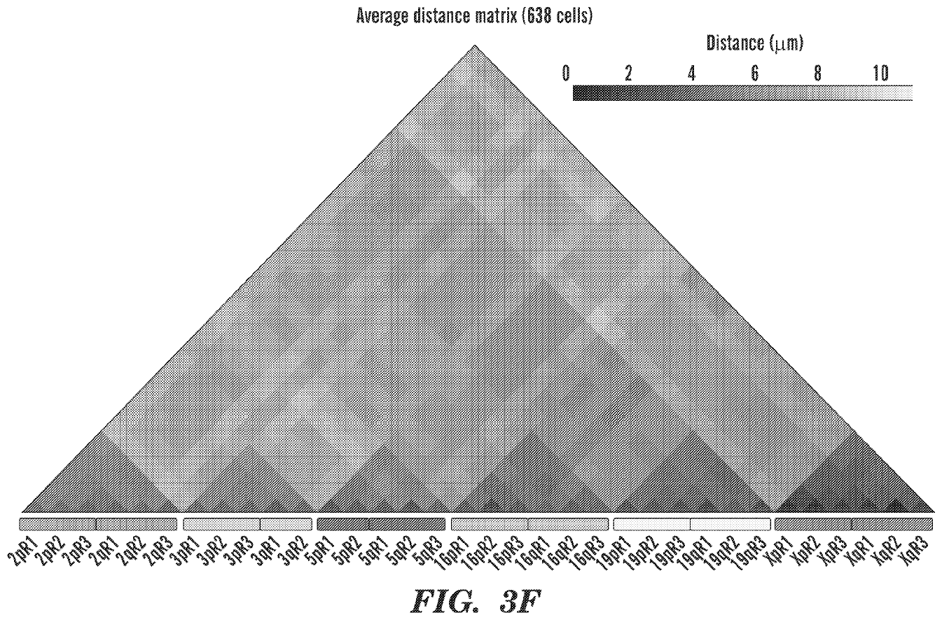

Four rounds of O-LIT successfully identified 100% of the 66 36plex-5K targets in interphase cells (n=2 cells from two replicates; data not shown). To amplify target signal and increase identification, 36plex-5K contained the same barcode on both Mainstreet and Backstreet, allowing simultaneous sequencing off of both streets (MSBS) (see e.g., FIG. 2C). Nuclei were decoded manually, as the algorithms used previously to decode single loci (Chr19-20K, see e.g., FIG. 1A-1F) were unable to accommodate the range of target signal intensity size that were encountered in these studies. Manual decoding, however, does not scale well, with each nucleus requiring up to 3 hours to decode. Thus, an automated pipeline was developed for image analysis that can address a wide range of signal intensities and sizes by interrogating every pixel individually (see e.g., FIG. 3A). Briefly, this approach begins by aligning images from all sequencing rounds, generating histograms of signal intensities for each species of fluorophore, and then normalizing the channel's histograms, such that the pixel intensities can be compared across the different fluorophores. Next, each pixel of every image of each round of sequencing is analyzed, tracked, and decoded as an individual unit (see e.g., Lee et al. 2014, supra; Lee et al. 2015, Nature Protocols 10 (3): 442-458; the content of each of each is incorporated herein by reference in its entirety), after which the resulting barcode for every pixel is matched to the list of all possible barcodes, and positive matches are mapped. Pixels having the same barcode that are also contiguous are then grouped to form 3D pixel patches.

The “every-pixel” approach has advantages over centroid-based approaches, where signals that span multiple pixels are tracked via the central positions of the signals. First, it is more tolerant of the imperfect alignment of multi-pixel signals because the likelihood that at least one pixel from one round of sequencing will overlap with a pixel from another round is greater than that of centroids overlapping. Second, because the spatial resolution afforded by the every-pixel approach allows for the identity of non-overlapping pixels to be preserved, it is better be able to resolve the positions of genomic targets that, due to the densely packed nature of DNA within the nucleus, are closely positioned in 3D space (see e.g., FIG. 3A; targets 2pR1 and 2pR2).

The every-pixel pipeline detected a high proportion (95±5.15%) of 36plex-5K targets in 2N cells, but with a high number of false positives (FPs) (582 FPs per nucleus; n=638 cells from 13 replicates; data not shown). To filter out FPs, a two-tier system was developed, wherein, first, Tier 1 filtered out pixels below a minimum signal intensity and/or patch size. Tier 1, alone, reduced FPs over 166-fold (3.49 FPs; 5.29%) while detecting 62.1±6.68% of targets (˜41/66) in each nucleus (638 cells from 13 replicates). In Tier 2, requirements for pixel intensity and patch size were lowered and then barcode subsampling was applied, with all newly detected signals filtered to be within 4.5 μm of Tier 1 targets from the same chromosome. This proximity-based filtering reflected the propensity of chromosomes to occupy separate territories, as well as prior measurements of the distance between consecutive Tier 1 loci detected along a single chromosome (data not shown; Methods). Tier 2 eliminated all FPs while detecting 80.2±7.3% (˜52/66) of targets in each nucleus with at least 60% of targets recovered in 75% of cells (data not shown). From the Tier 1 data, it was determined that the three least detected targets all had below average Oligopaint oligo density (<5.88 oligos/kb), indicating that target density can be more important than target size for detection. Target density along with other insights gained from the initial proof-of-principle experiments are critical in increasing the automated analysis pipeline to levels comparable to that of manual measurements. Importantly, O-LIT is reproducible and robust, as thirteen replicates using PGP1f cells produced similar ranges of barcode recovery (data not shown).

### Fine Scale Genome Organization at Single Cell Resolution

O-LIT mapping of 36plex-5K yielded highly detailed, single cell resolved spatial genomics data (see e.g., FIG. 3C-3E). Consistent with previous studies, the smaller chromosomes (e.g., Chr16 and Chr19) and larger chromosomes (e.g., Chr2, Chr3, Chr5) preferentially localized towards the center and periphery of the nucleus, respectively (data not shown). O-LIT permitted the tracing of individual chromosome 3-D paths (see e.g., FIG. 3D). Homolog resolved distance matrices were generated by measuring the pairwise 3-D spatial distance between each of the 66 targets. O-LIT permits the single cell comparisons of distance matrices and traces (see e.g., FIG. 3E). The single cell data can also be aggregated, producing average distance matrices between targets reminiscent of Hi-C heatmaps (see e.g., FIG. 3F). Here, homologous targets were grouped, due to the parental origin of each homolog being unknown. Hi-C matrices were derived of the same 36plex-5K regions from PGP1f cells (see e.g., Nir et al. 2018, PLOS Genetics 14 (12): e1007872) and compared them to a population averaged distance matrix from 638 cells, finding a high degree of correlation between the two separate methods (r²=0.705; Fig. SX). Two advantages of O-LIT (e.g., compared to Hi-C) are, first, its ability to map genomic loci without the requirement that loci of interest are proximal enough to be cross-linked together. Secondly, 0-LIT's ability to yield both single cell, homolog resolved as well as bulk population data. Specifically, multiple targets can be analyzed in the same nucleus. Thus, 0-LIT is a useful tool to interrogate genome structure-function relationships at multiple levels.

OligoFISSEQ Exact Barcode LIT (eLIT)

Described herein are LIT reagents that both render LIT more accessible as well as improves signal detection. These reagents are described below, beginning with a brief description of SOLiD chemistry.

SOLiD chemistry reads sequences as dinucleotides and, thus, it uses ˜1,024 species of fluorophore-labeled 8-nt oligos representing all 16 possible dinucleotide combinations of G, A, T, and C. While all ˜1,024 species of oligos are necessary in the context of de novo genome sequencing, a pool of oligos of this complexity is excessive for targeted approaches, such as those of OligoFISSEQ, where barcodes are defined by the user. As such, the complexity of the oligo pool for LIT was reduced by using just enough barcodes (JEB) to permit a more exact form of LIT referred to herein as eLIT. Without wishing to be bound by theory, it was reasoned that JEB could increase signal over background (see e.g., FIG. 4A). While eLIT is compatible with a variety of barcode configurations, the application demonstrated herein uses barcodes consisting of 5-bits each, wherein each bit is one of only four distinct 5-nt sequences. These bits are sequenced by ligation with pools of four species of 8-nt oligos, the 3′ end of each being perfectly complementary to one of the 5-nt bits and retained after cleavage and removal of the remaining 3 bases after ligation (see e.g., FIG. 4B). To further reduce the complexity of the pool of 8-nt oligos, the “universal base” deoxyinosine was used, rather than a mix of traditional nucleotide bases in positions 6, 7, and 8. In short, JEB reduced the pool of labeled oligos from −1,024 to 4. Application of OligoFISSEQ eLIT (0-eLIT) to Chr19-9K yielded a 3.3-fold brighter SBR as compared to the application of LIT to the same the same library using SOLiD oligos (n>55 nuclei; data not shown).

To test O-eLIT's ability to detect smaller targets, a library was designed similar to 36plex-5K (data not shown). The same 36 regions were targeted, but at finer genomic resolution (an average of 173 kb vs 876 kb per target) by using only the first 1,000 of 5,000 Oligopaint oligos from each target in the 36plex-5K library. For example, for target 2pR1, 36plex-5K targets the locus spanning 1,002,895-1,660,898 (˜658 kb), whereas 36plex-1K targets 1,002,895-1,147,495 (144 kb). To benchmark the library against 36plex-5K, the same barcodes were adopted for 35 of the 36 targets, the exception being the barcode of 5pR3, where it had previously shared the same barcode as 3qR3 as to test barcode detection between separate loci. O-eLIT sequencing of this library, called 36plex-1K, using only the Mainstreet yielded a barcode recovery that was higher than that obtained with O-LIT (SOLiD reagents) after 5 rounds (Tier 2 detection: 76.4±8.6%, n=439 cells from 8 replicates vs.
54.6%, n=41 cells, see e.g., FIG. 4C-4D). O-eLIT of 36plex-1K library also generates homolog resolved, single cell spatial data across all the targeted chromosomes (data not shown).

### Fine ChrX Path Tracing.

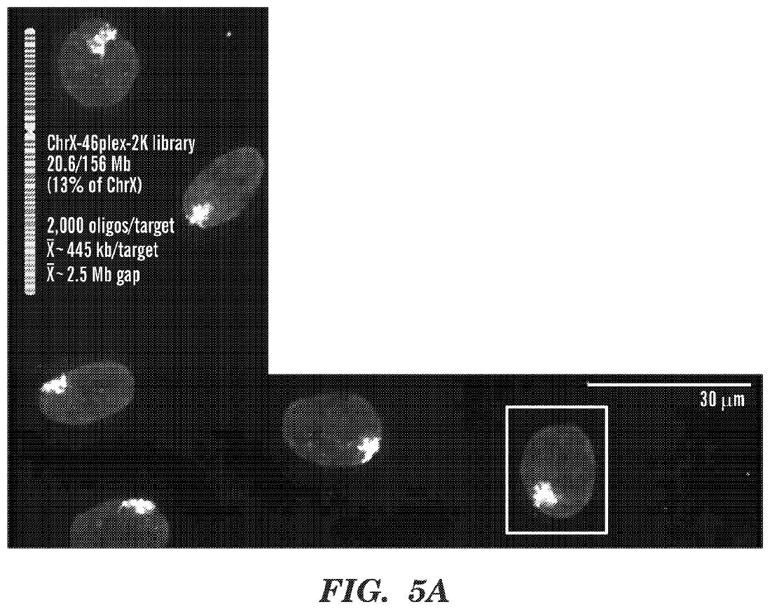

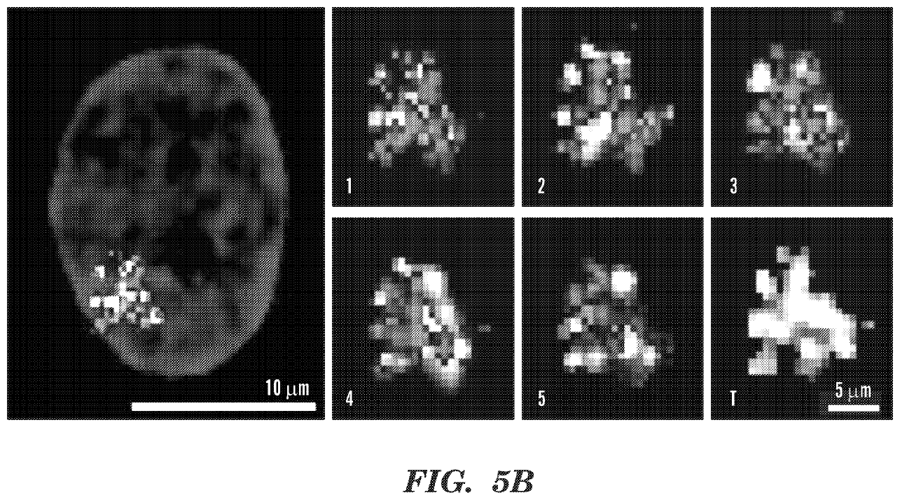

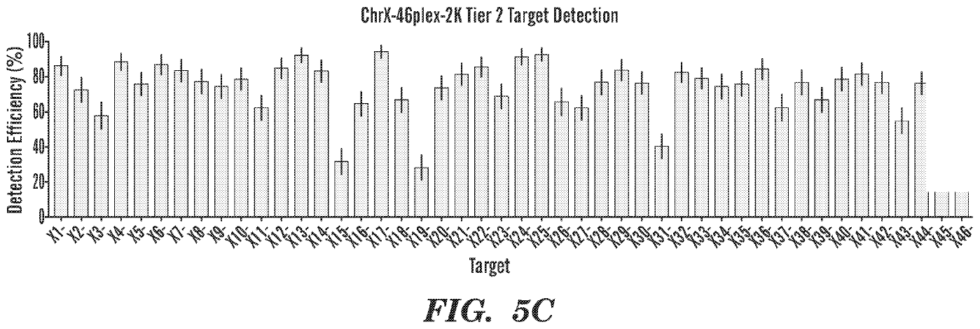

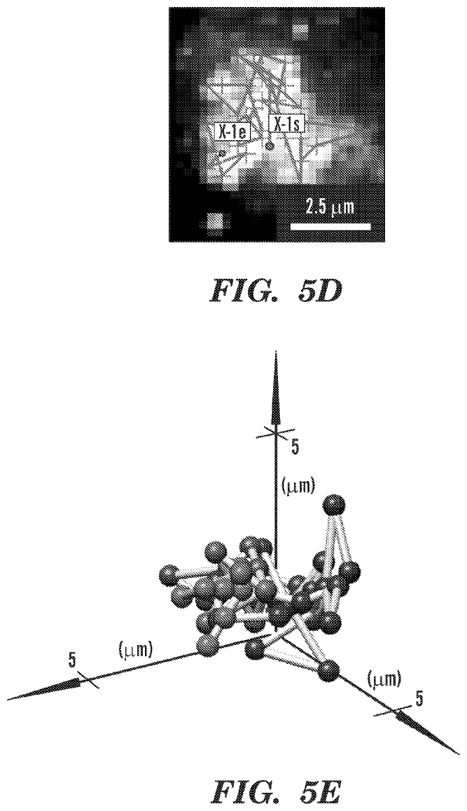

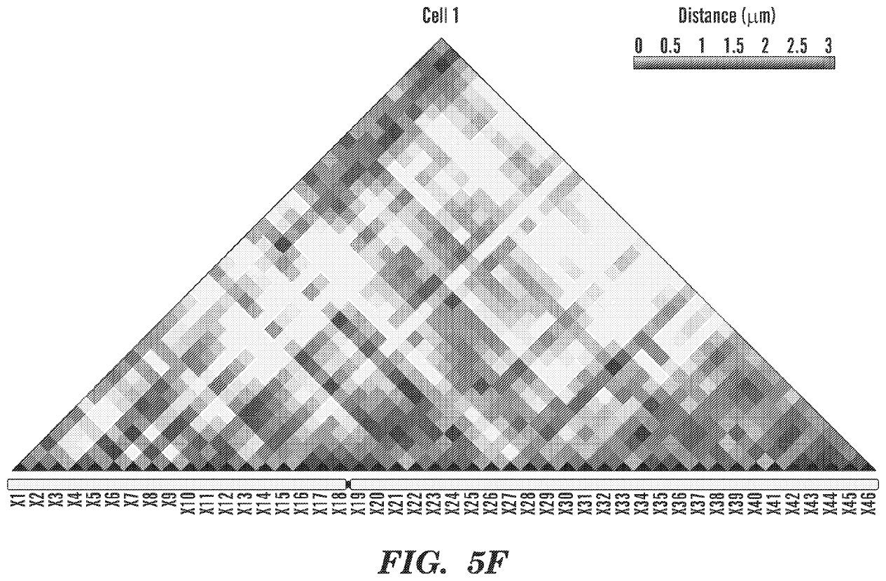

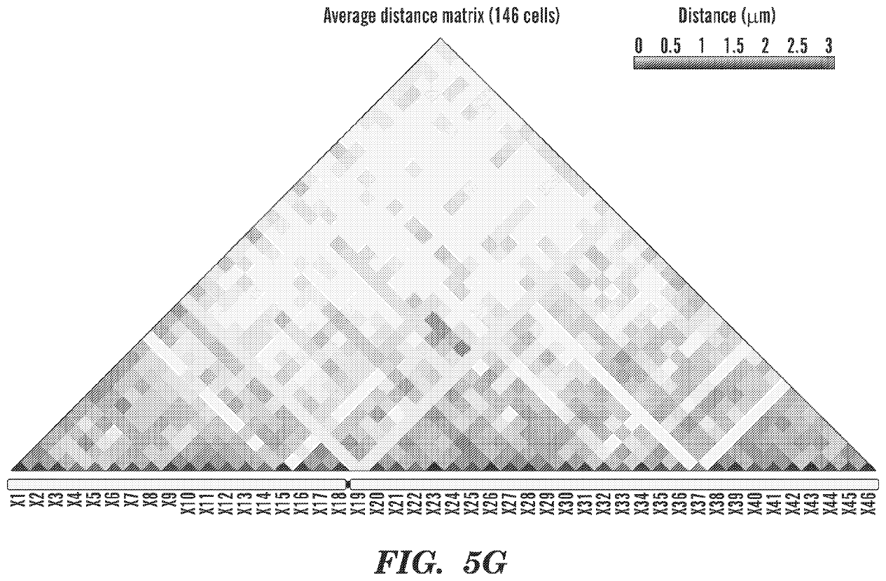

O-eLIT can trace chromosomes with finer genomic resolution, e.g., by applying an Oligopaint library (ChrX-46plex-2K) that targets 2,000 oligos to each of 46 regions along the human X chromosome, wherein the targeted regions range in size from 253 kb to 1.22 Mb (445 kb average), the average distance between targets is 2.75 Mb, and the total coverage of the X chromosome is 20.4 Mb or 13.3% (see e.g., FIG. 5A). As such, ChrX-46plex-2K served as a highly informative proxy for assessing the capacity of OligoFISSEQ to accommodate all other chromosomes. O-eLIT sequencing off of Mainstreet or both Mainstreet and Backstreet yielded comparable barcode recovery (data not shown), thus the data obtained from both sets were combined. Tier 2 detection recovered an average of 74.6±2.5% of targets (˜34/46; n=146 nuclei from 2 replicates) in male PGP1f cells (see e.g., FIG. 5B-5C). X19 could not be recovered after Tier 1 and could only be recovered 28% of the time after Tier 2. X19 targets a region adjacent to the centromere on the Q arm, suggesting an inhibitory feature of this area towards Oligopaint probes. To approximate the location of undetected targets, the 3D Euclidean distance was calculated between targets detected after Tier 2 and then assigned undetected targets at distances proportionately spaced along imaginary lines connecting targets detected on either side. This strategy for interpolation permitted the generation of high resolution traces of the X as well as distance matrices (see e.g., FIG. 5D-5F). O-eLIT also detected and traced PGP1f cells in which two X chromosomes were present; such cells could reflect aneuploidy for the X or G2 cells in which sister X chromosomes had separated (data not shown). Finally, as ChrX-46plex-2K was designed to allow Mainstreet and Backstreet to be sequenced independently, it can effect a ten-bit barcode that has the potential to distinguish over a million targets (104=1,048,576) (data not shown).

### OligoFISSEQ Applications

Finally, presented herein are additional applications of OligoFISSEQ and combinations with other technologies, including but not limited to single gene assays as well as combinations with immunofluorescence (IF), and super-resolution microscopy (see e.g., FIG. 6A-6D).

OligoFISSEQ-eLIT was used to sequence and map six genes, ranging in size from 11 kb to 100 kb (see e.g., FIG. 6A-6B). Mapping specific gene panels involved in certain pathways can be highly informative in fields such as development and cancer.

OligoFISSEQ-LIT was next combined with IF, permitting the study of both genome structure as well as protein localization in single cells (see e.g., FIG. 6C). Such techniques can be used for the construction of human cell atlases. Combining OligoFISSEQ and IF thus permits exploration of the spatial relationship between the genome and proteins.

Lastly, OligoFISSEQ improves the efficiency with which genomic regions can be imaged at super-resolution. More explicitly, because OligoFISSEQ can be carried out with widefield microscopy, it can image hundreds to thousands of cells in a single experiment and thus provide statistical power to analyses for which resolutions ≥˜250 nm are sufficient. This advantage of OligoFISSEQ is in contrast to that of super-resolution imaging methods using single-molecule localization microscopy, such as OligoSTORM (see e.g., Beliveau et al. 2015, supra), which, although capable of providing images of the genome with precisions reaching tens of nm in XY and hundreds in Z, are limited in terms of the number of cells that can be imaged. Thus, 0-LIT was combined with OligoSTORM to produce 40±5 and 60±5 nm in XY and Z resolution images of six genomic regions with only a single round of single-molecule localization microscopy. In particular, the Chr2-6plex-5K library was applied and bridge oligos were introduced containing binding sites for secondary oligos labeled with Alexa Fluor 647. The library targeted 6 regions on chromosome 2 to 20 PGP1f cells. One round of OligoSTORM was then performed to yield super-resolution images of the six Chr2 targets (see e.g., FIG. 6D), after which O-LIT primers were introduced. The nuclei were then imaged with two rounds of O-LIT to decode the six targets. That is, a single round of OligoSTORM followed by 2 rounds of O-LIT identified and super resolved 92.8% of the targets on Chr2 (n=7 cells). In some embodiments of any of the aspects, OligoFISSEQ can be performed first, such that proximal targets can be resolved by sequential rounds of OligoSTORM. Combining OligoSTORM and O-LIT is a powerful technique, as a single step of OligoSTORM can provide super resolved images of any discrete genomic region that has been identified by 0-LIT.

### Discussion

In conclusion, described herein is OligoFISSEQ, a suite of methods for in situ genome mapping. As described herein, OligoFISSEQ was used to map 66 genomic loci across six chromosomes, as well as finer chromosome tracing of 46 regions along the X chromosome. Together, these experiments demonstrate the ability to scale up OligoFISSEQ to map whole genomes and trace chromosomes at high resolution. As OligoFISSEQ uses widefield microscopy, a single experiment can yield thousands of cells, permitting further insights into the relationship between genome organization and function. Spatial transcriptomics can also be incorporated into OligoFISSEQ methods, such that panomic (e.g., genome, transcriptome, and proteome) spatial maps can be generated. Analysis of single cells at scale allows interrogation of genome organizational variation and functional consequences. Especially interesting is the degree in which the genome is organized. On one hand, heterogeneity in genome structure has been observed at multiple scales. Conversely, chromatin structural signatures may also exist, possibly similar to chromatin histone modifications for specific regions. The OligoFISSEQ methods described herein can visualize the genome at various scales, in numerous cells and tissues, under various conditions.

### Materials and Methods

Oligopaint library design: Oligopaint FISH probes were computationally designed for optimal hybridization as well as high specificity. Oligopaint genome binding sequences were obtained from the Oligopaints website (e.g., available on the world wide web at oligopaints.hms.harvard.edu; see e.g., Beliveau et al. 2018, supra), using hg19 genome and balanced settings. Genome homology sequences ranged from 35-41 nt. Universal forward and reverse priming sequences were appended to each Oligopaint oligo using OligoLEGO (e.g., available on the world wide web at github.com/gnir/OligoLego), allowing the libraries to be PCR amplified and renewable. The universal priming sequences also served as various OligoFISSEQ primer/bridge sites. Each library used herein was designed with specific features and is described in detail for each set.

Ligation based Interrogation of Targets Oligopaints: For Chr19-20K library, a portion of the universal forward priming sequence was used as the LIT primer binding site, followed by the LIT barcode. Barcode and color-code designation is as follows: 4=Cy5/Alexa-647, 3=TxRd, 2=Cy3, 1=FITC/Alexa-488.

36plex-5K library shared the same universal forward priming sequence and contained chromosome specific universal reverse priming sequences. Using the universal reverse priming sequence, individual chromosome targets could be amplified, hybridized, and detected. Universal forward priming sequences were used as LIT primer binding site. The LIT primers used were 18-nt. In cases where O-LIT was performed off of both Mainstreet and Backstreet, a LIT primer binding site was appended onto the Backstreet. Barcodes were specified using sequences from OligoLego. Candidate barcodes sequences were decoded to reveal color-code. To maintain color-code diversity between neighboring targets, barcodes were manually assigned to targets (e.g., barcodes were specified so that neighboring targets would have different colors in the first round). Each LIT barcode bit requires a 5-nt sequence, while the last barcode bit requires 8-nt to allow adequate space for 8mer binding. Thus, a 4-bit barcode requires 23-nt in total. For 36plex-5K, targets 3qR3 and 5pR2 contained the same barcode sequences to assess barcode recovery from separate genomic targets.

JEB/O-eLIT barcodes: For 36plex-1K, ChrX-23plex-Odd, ChrX-23plex-Even (ChrX-46plex-2K is a combination of ChX-23plex-Odd and ChrX-23plex-Even). 36plex-1K library targets targeted a subregion of 36plex-5K targets, with 1,000 Oligopaint oligos per target instead of 5,000. Additionally, 36plex-1K targets contained JEB compatible barcode bits. 36plex-1K targets contained the same barcode bit color code as 36plex-5K, with target 5pR3 as the exception.

ChrX-46plex library were designed to span the entire human X chromosome with 2,000 Oligopaint oligos per target. The library was divided into two sub-libraries (ChrX-23plex-odd and ChrX-23plex-even), with each sub-library targeting the odd (X1, X3, X5, etc.) or even targets (X2, X4, X6, etc.). Each sub-library contained the same universal forward priming sequences and different universal reverse priming sequences. ChrX-46plex barcodes contained JEB bits and were also manually assigned to maintain color-code diversity between neighboring targets.

The 6 gene library shared the same universal forward priming sequence and different universal reverse priming sequences. Barcodes were manually specified using JEB bits.

Synthesis based Interrogation of Targets barcode: For the Chr19-20K library, the universal reverse priming sequence was used as the SIT primer binding site, followed by the SIT barcode sequence. Barcode and color-code designation is as follows: 4=Cy5, 3=Cy5+Cy3, 2=Cy3, 1=blank.

For 36plex-1K, universal reverse priming sequence was used as SIT primer binding site, followed by SIT barcodes. Target color-code was designed to be the same as 36plex-5K but with SIT reagents.

Hybridization based Interrogation of Targets barcode: For Chr19-20K library, bridging oligos (HIT bridge) were designed to hybridize to Mainstreet and Backstreet. HIT bridges contained binding sites for HIT readout oligos. HIT readout oligo sequences were derived from OligoLego. Barcode and color-code designation is as follows: 0=blank, 1=Alexa 647/Cy5, 2=Cy3B/Cy3, 3=FAM/Alexa 488.

For 36plex-5K library, HIT bridges were designed to hybridize to Street specific sequences for each target. This was done by designing bridges flanking universal priming sites (forward and reverse) as well as 5′ or 3′ end of LIT barcodes, due to similar LIT barcodes being present on both Streets. HIT bridges contained binding sites for HIT readout oligos derived from OligoLego.

Oligopaint probe synthesis: Oligopaint oligos were purchased as single stranded Oligopools from CustomArray™ (e.g., available on the world wide web at customarrayinc.com/oligos_main.htm) or Twist Biosciences™ (e.g., available on the world wide web at twistbioscience.com) in 12K and 92K chip formats. Oligopools were amplified as previously described (see e.g., Nir et al. 2018, supra; Beliveau et al. 2017, supra) with minor modifications. Briefly, PCR conditions for each library and sub-library were optimized using real-time PCR (Bio-Rad™) to obtain optimal template concentration, primer concentration, and annealing temperature (see e.g., section on Real Time PCR). Next, libraries were linearly amplified with low-cycle PCR using Kapa Taq™ reagents. dsDNA PCR products were purified using Zymo™ columns and eluted with ultra-pure water (UPW). T7 RNA promoter sequence was then appended to Oligopaints using REV primers containing the T7RNAP on the 5′ end. Note that the T7RNAP can be added straight from the raw library. dsDNA PCR products were purified using Zymo™ columns and eluted with UPW. PCR products were then in-vitro transcribed using NEB™ HiScribe overnight at 37° C. to make RNA.

RNA products were reverse transcribed to make cDNA. RNA was then digested to leave ssDNA. This product was purified using Zymo™ columns. Final ssDNA Oligopaint oligos were resuspended at 100 uM in UPW and stored at −20° C. until use. Linear PCR, touched-up PCR, and ssDNA Oligopaint oligos were quality checked by running on 2% Agarose DNA gels to confirm single bands migrating at expected weights during synthesis.

Other oligonucleotides: Secondary fluorophore labeled oligos, LIT sequencing primer, SIT sequencing primer, JEB oligos, and MIPs were purchased from IDT™. HIT secondary oligos were purchased from Biosynthesis™. Alexa405 activator fluor was purchased from Thermo™.

Cell culture: Three human cell lines were used: PGP1f, IMR90, and MCF7. PGP1f are primary human fibroblast from male donor PGP1 (Corriell™; GM23248). They were previously found to be of normal karyotype (see e.g., Nir et al. 2018, supra). PGP1f were cultured in DMEM (Gibco™) supplemented with 10% Fetal Bovine Serum (Thermo™), 1× Penicillin-Streptomycin (PS, Thermo™), and 1×Non-essential amino acids. PGP1f cells were cultured for no more than 5 passages before thawing new cultures. IMR90 are primary human fibroblast from female donor (ATCC™, CCL-186). IMR90 were cultured in DMEM (Gibco™) supplemented with 10% Fetal Bovine Serum (FBS), 1× Penicillin-Streptomycin. MCF7 are cancer cell line from female breast cancer (ATCC™, HTB-22). MCF7 were cultured in DMEM (Gibco™) supplemented with 10% Fetal Bovine Serum (FBS), 1× Penicillin-Streptomycin (PS). Cells were cultured in 37° C. incubator at 5% C02.

Sample preparation for OligoFISSEQ: Ibidi Sticky Slide VI™ (e.g., available on the world wide web at ibidi.com; Cat. No: 80608) were used for all experiments except for metaphase spreads (FIG. 2B) and hydrogel (data not shown). Ibidi™ slides were assembled and allowed to cure overnight at 37° C. prior to use. Each well requires 100-200 uL of reagent and one hole was generally designated as the inlet and the other hole as the outlet. PGP1f cells from ˜70% confluent 10 cm dishes were detached from dishes using 1 mL trypsin, neutralized with 2-3 mL fresh media. 100 uL of cells in suspension were added to each Ibidi™ well and allowed to adhere and recover overnight at 37° C. incubator. The following day, media was aspirated, and wells were washed with 1×PBS and fixed for 10 min with 4% formaldehyde (EMS™) in final concentration of 1×PBS (Gibco™). Fixative was removed and cells were rinsed with 1×PBS. Cells were then permeabilized with 0.5% Triton™ (Sigma™) in 1×PBS final for 15 min on a rotator. Permeabilization reagent was aspirated and cells were rinsed in 0.1% Triton™/1×PBS and stored in this or PBS at 4° C. until use. Samples were used within 2-3 weeks after fixation.

Cell samples for MIP/hydrogel experiments were grown on rectangular glass microscope slides (VWR™). Cells were plated similarly to Ibidi™, except 150 uL of cells in suspension were plated onto discrete areas on rectangular slides (previously etched with glass etching pen to note the region) and incubated overnight at 37° C. incubator in petri dish. The following day, the same steps as with Ibidi™ above were performed but in 50 mL coplin jars. Cells were stored in 1×PBT in coplin jars until use.

Metaphase spreads were purchased from Applied Genetics (Product: HMM).

DNA FISH: Step by step protocols are adapted from (see e.g., Beliveau et. al. 2018, supra) and based on (see e.g., Pardue et al. 1969, PNAS 64 (2): 600-4; Bauman et al. 1980, Experimental Cell Research 128 (2): 485-490). All OligoFISSEQ methods begin with hybridization of primary Oligopaint libraries overnight and then deviate. Common to LIT, SIT, and HIT with Ibidi™ slides (all steps done on rotator unless specified): Ibidi™ wells washed with 0.1% PBT at RT for 5 minutes and incubated with 0.1 N HCl for 8 minutes. 2×SSCT washes were performed. Cellular RNA was digested with 50 uL of 2 ug/mL RNase A (Thermo™) in 2×SSCT for each well. Slide was incubated in 37° C. humid chamber (see e.g., notes below) for 1 hour. RNAse A was washed out by adding 2×SSCT. Pre-hybridization began by adding 50% formamide/2×SSCT for 10 min at RT. Pre-hybridization continued with prewarmed (60° C.) 50% formamide/2×SSCT being added and by placing the slide on top of heat block set in 60° C. water bath for 20 min. Next, the primary Oligopaint library was added. The samples were aspirated and 50 uL total of the primary Oligopaint oligo library (2 uM final) were added in hybridization mix (50% formamide, 2×SSCT, 10% Dextran Sulfate). Samples with primary Oligopaint oligo libraries were then denatured, wells were sealed with parafilm to prevent evaporation and slide was placed on pre-heated hot block in 80° C. water bath for 3 minutes under the weight of a rubber plug. Oligopaint oligo library hybridization to samples was performed by placing samples in humid chamber at 42° C. incubator for >16 hours. The next day, probes that did not hybridize were washed out by adding prewarmed (60° C.) 2×SSCT directly to each well containing primary hybe mix and aspirated. New prewarmed 2×SSCT was added and samples were incubated on hotblock for 15 min. This was repeated one time and then another time at room temperature (RT). After this wash is where the protocol deviates for the techniques, as described below. Note that cellular DNA was stained after every 2 rounds of sequencing to maintain adequate DAPI signal.

For detection of OligoPAINTS via secondary hybridization, samples were then prepared for secondary oligo hybridization to primary oligo streets for detection. Samples were washed with 30% formamide/2×SSCT for 8 min and 50 uL total of secondary oligos and/or bridge oligos were added at 1.2 uM in 30% formamide/2×SSCT to each well. Samples were incubated in humid chamber for 45 min at RT dark. Non hybridized secondary oligos were washed out with 30% formamide/2×SSCT being added directly in, aspirated, and incubated 2×15 min on rotator. Samples were washed with 2×SSCT two times 5 min. In some experiments, DNA was counterstained with DAPI in PBS for 10 min. Samples were then washed with 1×PBS ×2 5 min and imaged in 1×PBS or imaging buffer.

For cells on rectangular slides, the same overall protocol as above was performed but in coplin jars, scaling wash volumes accordingly (e.g., 25 μL volumes for primary and secondary hybridizations). The protocol is modified as follows: RNAse was added directly to cells on rectangular slide and covered with 22×22 mm coverslip. Post RNAse washes were performed by transferring slide and coverslip to coplin jar and “sliding” the cover slip off Same approach was performed for secondary hybridization. Primary Oligopaint hybridization was performed by adding primary Oligopaint mix directly to cells on rectangular slide, covering with 22×22 mm coverslip, and sealing edges with rubber cement (Elmer's™). Rubber cement was allowed to dry for 3 min and sample was denatured on heat block, similar to Ibidi™.

Ligation based Interrogation of Targets (LIT): LIT is built upon Oligopaint (see e.g., Beliveau et al. 2012, supra), SBL (see e.g., Shendure et al. 2005, supra), and FISSEQ technologies (see e.g., Lee et al. 2014, supra). After hybridization of the primary Oligopaint library, for O-LIT, samples required treatment with phosphatase to deplete endogenous phosphates that could prime ligation, contributing to background and poor signal. The samples were washed with 50 uL of 1× NEB CutSmart™ buffer for 8 min. Next, 50 uL of shrimp alkaline phosphatase (rSAP; NEB™) was added to each well and incubated at 37° C. humid for 1 hour. To inactivate the phosphatase, sample was then transferred to pre-heated heat block in 65° C. water batch for 5 min, and washed ×2 with preheated (65° C.) 2×SSCT on heat block for 5 min each. RT 2×SSCT was added for 5 min. Samples were then prepared for LIT primer binding by washing with 30% formamide/2×SSCT for 8 min and 50 uL total of LIT sequencing primer was added at 1.2 uM in 30% formamide/2×SSCT to each well. Samples were incubated in humid chambers for 45 min. Non-hybridized LIT primers were washed out with 30% formamide/2×SSCT being washed directly in, aspirated, and incubated 2×15 min on rotator. Samples were washed with 2×SSCT two times 5 min. Next, samples were prepared for first round of LIT by adding 100 uL of 1× Quick Ligation™ buffer (NEB™) for 8 min and aspirated. LIT reaction mix was prepared on ice. Before adding ligases (added last), vigorous vortexing was performed on the LIT reaction mix. After vortexing, ligases were added and mixed thoroughly by pipetting. O-eLIT reagent was performed similarly but instead of SOLiD™ purple reagent mix, 40 pmol of each JEB oligo was added and UPW was adjusted accordingly. 100 uL of this mix was added to each well and samples were incubated in humid chamber at 25° C. for 55 min. LIT reaction mix was then aspirated and samples were rinsed with 1M GHCL, and washed ×2 15 min on nutator at RT. 1×PBS wash 5 min was performed. Cellular background fluorescence was reduced by treating the samples with 100 uL True Black™ (Biotum™) in 70% EtOH for 2 min. 3×1×PBS quick rinses, and then 10 min wash was performed. Samples were then imaged in 1×PBS or imaging buffer. Before proceeding to the next LIT round, non-ligated phosphates are treated with phosphatase (Quick CIP; NEB™) for 30 min at 37° C. Quick CIP is then washed out with 3×GHCL washes 5 min. Previous LIT round is cleaved to release fluorophore and regenerate 5′ PO₄ by rinsing and then 15 min incubation at RT rotator with Cleave 1, and then the same for Cleave 2. Samples are then rinsed ×3 with GHCL and washed ×2 5 min. The next round of LIT can proceed with the pre-ligation step. After the last barcode bit is read, the fluorophore can be cleaved and all targets can be detected by hybridizing specific bridges and fluorophores as in DNA FISH method.

Synthesis based Interrogation of Targets (SIT): SIT is based upon Oligopaint (see e.g., Beliveau et al. 2012, supra) and SBS (see e.g., Guo et al. 2008, supra) technologies. After hybridization of primary Oligopaint library, samples were then prepared for SIT primer binding by washing with 30% formamide/2×SSCT for 8 min and 50 uL total of LIT sequencing primer was added at 1.2 uM in 30% formamide/2×SSCT to each well. Samples were incubated in humid chambers for 45 min. Non-hybridized SIT primers were washed out with 30% formamide/2×SSCT being washed directly in, aspirated, and incubated 2×15 min on rotator. Samples were washed with 2×SSCT two times 5 min. First round of SIT proceeds by rinse with 100 uL of pre-warmed (60° C.) NextSeq Buffer X™, and then incubation on 60° C. heatblock in water bath for 5 min. Sample is aspirated and washed with 2×SSCT ×3 10 min. 1×PBS wash was performed and samples were imaged in 1×PBS, imaging buffer, or NextSeq™ imaging buffer. Before proceeding onto the next SIT round, sample is treated with NextSeq Buffer X™ with a rinse, then 5 min incubation on 60° C. heatblock in water bath. Sample is then washed 3×10 min in 2×SSCT. The next round of SIT can now proceed. For all target identification, SIT primers containing an Alexa488 can be used or secondary oligos with bridges can be added.

Hybridization based Interrogation of Targets (HIT): HIT is based on Oligopaint (see e.g., Beliveau et al. 2012, supra) and SBH technologies (see e.g., Lubeck et al. 2014, Nature Methods 11 (4): 360-61; Chen et al. 2015, supra; Eng et al. 2017, supra). After hybridization of the primary Oligopaint library, samples were then prepared for HIT bridge oligo hybridization to primary oligo streets for detection. HIT bridges for 36plex-5K were designed to span the universal priming region and part of either the MS barcode or BS barcode. Samples were washed with 30% formamide/2×SSCT for 8 min and 50 uL total of bridge oligos were added at 1.2 uM in 30% formamide/2×SSCT to each well. Samples were incubated in humid chamber for 45 min at RT dark. Non hybridized bridge oligos were washed out with 30% formamide/2×SSCT being added directly in, aspirated, and incubated 2×15 min on rotator. The first round of HIT proceeds by addition of 50 uL to each well with round specific HIT secondary oligos at 1.2 uM of each in 30% formamide/2×SSCT for 45 min at RT dark humid chamber. Non hybridized HIT secondary oligos were washed out with 30% formamide/2×SSCT being added directly in, aspirated, and incubated 2×15 min on rotator. Samples were washed with 2×SSCT ×2 5 min and then 1×PBS for 5 min. Samples were imaged in 1×PBS or imaging buffer. Before proceeding to the next round, previous HIT round secondary oligo fluorophores are cleaved via rinse and incubation for 15 min with 1 mM Tris(2-carboxyethyl)phosphine (TCEP, Sigma™). 3×PBS rinse was performed and the next HIT round can proceed.

Immunofluorescence: To visualize proteins, samples were subjected to immunofluorescence. After OligoFISSEQ, Oligopaint oligos were removed by washing with 80% formamide/2×SSCT 2×7 min. Next, samples were washed with 2×SSCT for 3 min, rinsed with 1×PBS and fixed in 4% Formaldehyde/PBS for 10 min. After PBS rinses and permeabilization in 0.5% Triton/PBS for 10 min, samples were blocked in 3% BSA/PBT for 1 hr. Primary antibodies diluted in 1% BSA/PBT were then added to each well, sealed with parafilm, and incubated overnight (0/N) at 4° C. for >12 hrs. The next day, primary antibody was removed and 3×PBT washes were performed. Secondary antibodies diluted in 1% BSA/PBT were then added at 1:500 dilution for each for 1 hr at R/T shaker. Wheat Germ Agglutinin (WGA, 1:20) could also be added during the secondary incubation step. 3×PBT washes for 5 min each were performed, and samples were restained with DAPI (1:1000) for 10 min and imaged in imaging buffer.

Hydrogel: Hydrogel embedding was based on (see e.g., Moffitt et al., Proc Natl Acad Sci USA. 2016 Dec. 13, 113(50):14456-14461). Cells for hydrogel embedding were grown on rectangular glass slides. FISH was performed on these slides as described in “DNA FISH” section. After primary Oligopaint library hybridization, samples were washed in 60° C. 2×SSCT for 20 min, followed by a 10 min wash at RT, then 1×PBS for 5 min. In preparation for hydrogel embedding, slides were air dried for 5 min and area around cells was wiped dry with Kimwipe™. Hydrogel reagents were combined in eppendorf tubes on ice and degassed on ice in vacuum chamber during incubations. Cells were then washed for 10 min at 4° C. with hydrogel mix without APS/TEMED. Hydrogel mix was then removed from sample and ˜20 uL of hydrogel solution was spotted onto parafilm on gelation chamber slide (rectangular slide wrapped in parafilm, using 2 22×22 mm coverslips as spacers on each end of the slide; data not shown). Slide sample was then flipped onto hydrogel solution/gelation chamber, being careful to spread the hydrogel solution without forming bubbles. Sample was then incubated at 37° C. for 1 hr in vacuum chamber. After incubation, gelation chamber was carefully removed. Edges of hydrogel disc were trimmed and diamond etching pen was used to break rectangular slide, preserving the gel/glass slide portion. Gel/glass slide portion was then transferred to 35 mm petri dish and digested in 2 mL digestion buffer (recipe from Moffitt et al. 2016, supra) O/N at 37° C. After O/N digestion, cell/hydrogel dissociates from the glass slide so extra care should be taken to avoid hydrogel damage. Digestion buffer and glass slide is removed and hydrogel is washed in 2×SSC for 3×20 min. The hydrogel can be divided into smaller pieces for downstream applications. To note orientation, hydrogel pieces can be cut into distinct shapes, which permits easier downstream imaging and alignment. After cutting, the hydrogel sample can be transferred to 1.5 mL eppendorf tubes for easier handling.

Metaphase FISH: Steps were performed using coplin jars except for where noted. RNase A treatment was performed by adding RNase A and sandwiching under 22×22 mm coverslip and incubated in humid chamber. Primary Oligopaint hybridization was performed the same way.

Widefield Microscopy: OligoFISSEQ and diffraction limited FISH was imaging was performed using a widefield fluorescence setup. Used herein is a Nikon Eclipse Ti™ body equipped with a Nikon 60×1.4NA Plan Apo Lambda™ (Nikon MRD01605) objective lens, Andor iXon Ultra EMCCD™ camera (DU-897U: 512×512 pixel FOV, 16 μm pixel size), X-Cite 120 LED Boost™ light source, motorized stage, and off the shelf filter sets from Chroma™ (˜488 nm 49308 C191880, ˜532 nm 49309 C191881, ˜594 nm 49310 C191882, ˜647 nm 49009 C177216). Images were obtained with ND4 and ND8 filters in place. Microscope operation was handled by Nikon NIS Elements™ software. In general, z-stacks were obtained with 0.3 μm slices with 2-300 ms exposure time and 20-60% LED intensity, depending on library being imaged. XYZ stage position was maintained within nd2 metadata and was essential for returning to the same field of view (FOV). Orientation of sample into the stage and sample holder was carefully maintained as to permit returning to the same FOV. This was important, as the sample was removed after imaging and between sequencing rounds.

STORM imaging: In order to combine OligoFISSEQ with OligoSTORM, one round of STORM imaging was first performed on all the 6 targets on chromosome 2 inside a PGP1f male fibroblast cells by hybridizing ALEXA 647 labeled secondary oligos that bind to the bridges (present in the back streets of individual Oligopaint oligos targeting 6 spots on Chr 2) containing a binding site for secondary oligos. OligoSTORM samples were imaged on a Vutara 352™ biplane system with an Olympus™ 60×1.3NA Silicone objective (UPLSAPO60XS2). For single molecule blinking, a switching buffer was used containing 2-Mercaptoethanol and GLOXY (see e.g., Nir et al. 2018, supra). The excitation laser power was set at 60% on the software (6.3 kW/cm² at the objective) for the 640 nm laser and 0.5% on the software (0.08 kW/cm² at the objective) for the photoactivation laser of 405 nm. 30-40 Z slices of 0.1 μm thickness were used for each Z slice. 10-12 photoswitching cycles of 250 frames per cycle were used for each Z slice.

The STORM images were analyzed using Vutara SRX™ software (see e.g., Nir et al. 2018, supra). DBSCAN clustering algorithm was used to identify the clusters from the raw image. 50 particles within a 0.1 μm distance was used for clustering. The mean axial precision was 50+/−10 nm in Z and mean radial precision was 17+\−5 nm in XY. The resolution of the super-resolved structures was calculated by Fourier ring correlation analysis (a built up feature in SRX™ software). Resolution in XY was 40+/−5 nm and resolution in Z was 60+/−5 nm.

Data visualization: Images were processed using either Nikon Elements™ or ImageJ™ FIG. 2D was generated using ImageJ (Plugins>3D Viewer). Chromosome schematics were generated using ChromoMap™ (see e.g., Anand 2019, BioRxiv, April, 605600). Figures were assembled in Adobe Illustrator™. Micrograph images for publication figures were post-processed using Brightness and contrast enhancement (ImageJ>Image>Adjust>Brightness/Contrast).

### Example 2

Methods for Highly Multiplexed In-Situ Visualization and Identification of Targets

methods for mapping genomes in situ are limited. Spatial genomics, where genomic loci are localized inside the 3D (three-dimensional) nucleus, is an emerging field concerned with the fact the spatial localization of DNA plays a critical role in how it is expressed, repaired, replicates, and functions. Current genome visualization methods are challenged by throughput as well as target detection, due to sequential labeling schemes and signal generation. As spatial genomics is limited by the ability of conventional microscopes to detect 4-5 colors at a time, a majority of techniques rely on the sequential visualization of targets. This method scales linearly and is not realistic for visualizing all ˜25,000 human genes.

To overcome this challenge, a barcoding scheme where each target can be represented by a multibit color barcode. This method scales exponentially. 4 colors and 8 bits of barcode permits the identification of 65,536 targets (4\hat{ }8). Barcodes can be read by fluorescent in situ sequencing (FISSEQ) techniques.

Current applications of genome visualization via FISSEQ (e.g., OligoFISSEQ) utilizes SOLiD sequencing by ligation chemistry. This method relies on an accumulation of many Oligopaints (e.g., thousands) at each target to generate a detectable FISSEQ signal. This difficulty of this detection increases as the rounds of bit reading increases. An aspect of SOLiD chemistry that confounds the target signal detection is that the chemistry utilizes a pool of 1,024 8nt fluorescently labeled oligos to read barcodes (see e.g., FIG. 10C). While this allows a high level of flexibility, this flexibility is unnecessary for spatial genomics of targeted regions.

Presented herein is chemistry allowing for the generation of cleavable oligonucleotides. The oligos can be cleaved specifically between any nucleotide position, resulting in the generation of a 5′ phosphate. This 5′ phosphorylated oligo is then compatible for ligation with a 3′ hydroxylated oligo with DNA ligase (see e.g., FIG. 10A-10B). These modified oligos can also be functionalized with detectable labels (e.g., fluorophores), generating reagents that can be used for FISSEQ. Furthermore, this chemistry can be used to generate 8nt oligos with specific sequences at nt positions 1-5 and universal bases at positions 6-8. These modified oligos are cleavable between positions 5 and 6. These oligos can be used in OligoFISSEQ to sequence 5nt specific barcodes (see e.g., FIG. 10E-10H). Such an approach reduces the number of fluor-labeled oligos used for OligoFISSEQ from 1,024 to 4, dramatically increasing the sequencing signal (see e.g., FIG. 10C, FIG. 10F-10G). These oligos can be referred to herein as “Just Enough Barcodes” or “JEB”.

As shown herein, OligoFISSEQ can be simplified by designing barcodes containing specific 5nt bits on Oligopaints and utilizing 4 specific 8nt fluorescently labeled oligos to sequence them (see e.g., FIG. 10D). The resulting oligo sets and methods, referred to herein as JEB, are simplified, discard the unnecessary oligos contained in SOLiD chemistry, and result in higher sequencing signal. The sets and methods as described herein demonstrate at least two advantages compared to other methods: (1) they decrease in number of Oligopaints required to produce sufficient signal from a target, and (2) they increase the number of barcode bits that can be detected, increasing the number of targets that can be uniquely identified. Ultimately, sets and methods as described herein permit the imaging of the whole genome.

### Example 3

Not only is the genome vast, it is internally duplicated in the form of homologous chromosomes. And, although numerous studies have showcased its intricate internal 3D organizational structure, it is now clear that those structures sport a high degree of variability. Finally, and perhaps most remarkably, it functions as an integrated unit, coordinated in its organization, its replication and choreography through cell division, its inheritance, the regulation of its transcriptional activity, and even its demise during senescence. For these reasons, there is a growing need for technologies that can address the genome as a whole, distinguish homologous chromosomal regions, and also have the high-throughput that can lend the statistical power essential for extracting underlying patterns from a heterogeneous population. This need is as great for biochemical approaches as it is for our capacity to visualize the genome, visualization providing the capabilities that can reveal 3D genome organization. Described herein is the first proof-of-principle for OligoFISSEQ, a set of three methods for in situ genome mapping. These studies mapping 66 genomic loci across six chromosomes, providing a finer trace of 46 regions along the X chromosome, as well as accelerating the visualization of genome regions at super-resolution demonstrate the potential for scaling OligoFISSEQ towards mapping whole genomes. Importantly, OligoFISSEQ has the capacity to consistently identify the same targets across thousands of cells and is thus well-suited for addressing, at widefield-resolution as well as super-resolution, the challenge of variability, one of the most daunting as well as fascinating aspects of the genome. Although variability may represent a stochastic process, it may also be an essential signature genomic regions. While variability is thought of locally, its impact may reach globally. And, although it may appear random, that randomness may be under the exquisite control of a regulatory program that directs structural conformations, as much the outcome of evolution as any other honed genetic element.

Owing to the versatility of the streets of Oligopaints, OligoFISSEQ also has the capacity to meld with other technologies. For example, with minor adjustments to barcodes and image acquisition, OligoFISSEQ could permit multiplexed, possibly also multi-color, visualization of fine chromosome folding in combination with optical reconstruction of chromatin architecture (ORCA) (see e.g., Mateo et al. 2019), Hi-M (see e.g., Andr6 Cardozo Gizzi et al. 2019), and OligoDNA-PAINT (see e.g., Nir et al. 2018). In terms of scaling, at least 3,000 targets can be mapped in PGP1f nuclei, assuming distinct, non-overlapping signals, with the potential to increase that number through strategies that should enable us to decrease target size, sequence at super-resolution microscopy, or permit expansion microscopy (see e.g., F. Chen, Tillberg, and Boyden 2015). Temporal separation of sequencing (see e.g. (Mateo et al. 2019; Eng et al. 2019)) can also be used to resolve overlapping targets. In particular, temporal separation can be achieved through performing O-LIT for different subsets of targets at different stages, such that problematic targets are sequenced at different times.

The ability to visualize entire genomes, simultaneously, in many individual cells presents the opportunity to explore genome structures in unprecedented ways. Recent findings of structural and organizational variability at single-cell levels (see e.g., S. Wang et al. 2016; Bintu et al. 2018; Nir et al. 2018; Finn et al. 2019; Mateo et al. 2019; Andr6 Cardozo Gizzi et al. 2019) can particularly benefit from taking these “big-picture” genome-wide views. As OligoFISSEQ can be used to visualize the entire genome, this will allow researchers to visualize structures and their variability in an unbiased way. For example: minor, inconsequential perturbations in one part of the genome, may have a profound effect on the global scale, similar to what is referred to as the “butterfly effect” (see e.g., Lorenz 1963). Furthermore, spatial mapping at genome scale will create individual cell interaction maps that may reveal gene spatial networks, advancing our understanding of biology.

### Tier 1 Detection

Preprocessing. The widefield microscope dataset (z-stack) of each round of OligoFISSEQ contains 5 channels: Alexa 647, Texas Red, Cy3, Alexa 488 and DAPI and a series of z-slices. The z-stacks are deconvolved and background corrected using 20 iterations of the Richardson-Lucy algorithm using a theoretically calculated point spread function with Nikon software.

Rounds are compiled into hyperstacks composed by the 5 channels, a series of z-slices and one frame per round. If an image where all the punctas are labelled, like in toto image, is available it is included as a new additional frame. The hyperstacks are aligned using Fiji plugin “Correct 3D Drift” (see e.g., Parslow, Cardona, and Bryson-Richardson 2014). Images of DAPI stained nuclei are used to perform threshold segmentation and extract each individual cell from the initial image as a separate region of interest (roi). The segmentation provides information about location and envelope of individual nuclei that compose each hyperstack. Nuclei with areas below 25 μm2 are discarded.

Detection of barcodes. To compare intensities from different channels images are normalized by dividing its intensities by the maximum intensity among the values of all the z-slices in the same round and channel.

For the detection of barcodes and for each round the intensities of every pixel position is compared across different channels. The channel with the highest value is kept as the prevalent. At every pixel position the transition between channels along the different rounds is compared with the list of expected barcodes. A barcode is assigned to a pixel position if the set of transitions coincides with the one associated to the barcode. A maximum intensity projection (MiP) image is built by averaging the intensities of the prevalent channels of every round. Connected pixels having the same barcode are grouped to form 3D patches. The following information is collected and saved for each patch: barcode; center position; number of pixels forming part of the patch (size); maximum intensity of the pixels of the patch; pixel position having the maximum intensity of the pixels of the patch. If there is an image with all punctas labelled the information of the intensity of each pixel position is stored in an additional file.

### Tier 2 Detection

Chromosome tracing. Patches composed by a single pixel location are discarded. The rest of the patches are used in the tracing disregarding its intensity or size.

Patches with high intensity values are selected as the most confident and used to find the chromosome centers. Used herein is an implementation of the Constrained K-means algorithm to find the center of the set of barcodes belonging to the same chromosome. To separate the homologs, a cannot-link constraint was used in the two copies of the same loci to avoid having them in the same cluster. A sphere of radius 4.5 μm with origin in the centers was used to delimit the chromosome territory and filter out patches located outside.

The Domino sampler of the Integrative Modelling Platform (see e.g., Russel et al. 2012) is the core element of the chromosome tracing. In Domino each locus is represented by a particle with a finite set of different possible locations in the image. The locations are extracted from the list of patches having the same barcode as the one assigned to the locus. The remaining factors of the proposed problem are encoded in the system as restraints to the list of possible solutions. The following restraints are imposed to the system to filter compatible solutions: two particles cannot share the same location/patch; two consecutive particles of the same chromosome should be closer than a distance of 4 μm for the 36plex dataset and 1 μm for the ChrX-46plex; chromosomes must be confined in territories modelled as spheres of radius 4.5 μm.

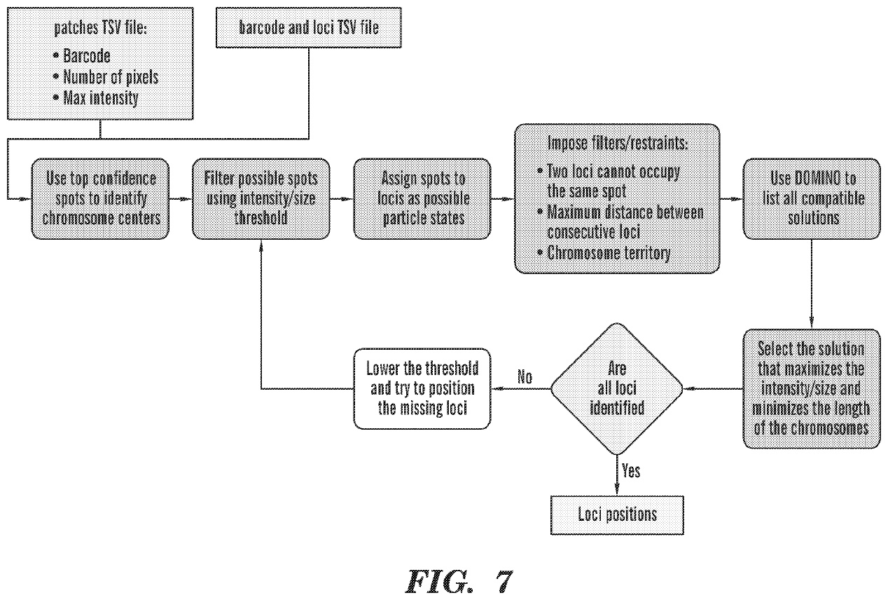

Chromosome territory and distance between consecutive loci are inferred as explained in section “Inferring chromosome territory and maximum distance between consecutive loci.” By applying these additional constraints to the barcodes, patches can be used having intensities that are below but not far from the detection thresholds (see e.g., Table 3) and are likely to be true positives. Patches with higher intensities and sizes are most likely to be true positive loci. Therefore, a score based on intensity and size is assigned to each patch as a measure of the likelihood of the patch to be a true positive detection. The list of patches is sorted by score and used as input data to an iterative process to find the most probable path of each chromosome (see e.g., FIG. 7).

The iterative process of tracing the chromosomes starts by assigning patches with high score to the corresponding loci. The process is executed one time per chromosome considering all homologs at the same time because barcodes are not designed to distinguish them. Domino is used to list all possible solutions that are compatible with the imposed restraints. Each solution has a total score obtained by the addition of the scores of the individual patches selected in that particular solution. The conformation having the highest total score is selected. In case two or more solutions yield an identical total score, the solution is selected which conforms the shortest chromosome spatial length. Loci assignment is done in an iterative process by lowering the threshold to use more patches as input and use the previous approach to select the remaining unassigned loci. This iterative process finishes when all loci have been identified or there is no more input data to feed Domino.

### Detection Efficiency and False Positives Ratios

To calculate the detection efficiency per barcode the datasets are filtered using intensity thresholds (see e.g., Table 3) that are optimized for every experimental condition. Patches formed by one single pixel are also discarded regardless of its intensity.

For the 36plex datasets the mean of barcodes detected per nuclei excluding the ones assigned to the X chromosome was calculated. In the ideal case and due to the ploidy, two barcodes are expected per nucleus. In reality the datasets may eventually include false positives or duplicates of patches that are probably belonging to the same oligo which will rise the ratio. Nucleus with a mean of more than 2.5 barcodes are discarded because they are most likely in a mitotic process. For the ChrX-46plex a similar procedure was followed and nuclei were discarded wherein the mean of detected barcodes was higher than 1.5.

For each of the remaining nuclei the ratio of detected versus expected barcodes was computed. Two barcodes per cell are expected per cell except for the barcodes belonging to the chromosome X. The ratios per barcode and per cell are capped at 1.0 and averaged over all the cells to produce the detection efficiency. For the False Positive ratio of the barcode, instead, the calculation comprises the excess of detections as the detected minus the expected value in the cases where detected is over expected, and then computation of the ratio excess versus expected.

### Distance Heat-Maps and Hi-C Maps

For every traced nucleus, all pairwise distances were calculated between the detected loci and averaged the result among all cells. For the average heatmap of the 36plex-5K LIT dataset loci 3qR3 and 5pR3 were not taken into consideration because they shared the same barcode and were therefore indistinguishable. Hi-C maps of PGP1f cells were obtained from previous in situ Hi-C experiments (see e.g., Nir et al. 2018). The values of the interaction frequencies in the included Hi-C maps were extracted from observed values of interaction matrices produced at 5Kbp resolution. The submatrices formed by the genomic regions of each pair of probes were aggregated to obtain the inter-loci observed interaction. Single cell heatmaps were built with the identification of homologous chromosomes. The list of barcodes is traced according to the procedure described in the methodology. Then all pairwise distances of the traced loci are calculated. Not identified loci appear as grayed columns/rows.

TABLE 2
List of correlations

| Library | No filtering | Filtering out mitotic cells |
|---|---|---|
| 36plex-5K LIT | pairwise distances = 2095723 | pairwise distances = 1287298 |
| | correlation = −0.685 | correlation = −0.705 |
| | pvalue = 4.789e−161 | pvalue = 1.771e−174 |
| 36plex-1K eLIT | pairwise distances = 1871099 | pairwise distances = 1077893 |
| | correlation = −0.692 | correlation = −0.697 |
| | pvalue = 1.167e−185 | pvalue = 1.091e−189 |
| chrX 46plex | pairwise distances = 370499 | pairwise distances = 180896 |
| | correlation = −0.562 | correlation = −0.64 |
| | pvalue = 8.041654826642585e−177 | pvalue = 7.0735300041298106e−245 |

### Inferring Chromosome Territory and Maximum Distance Between Consecutive Loci

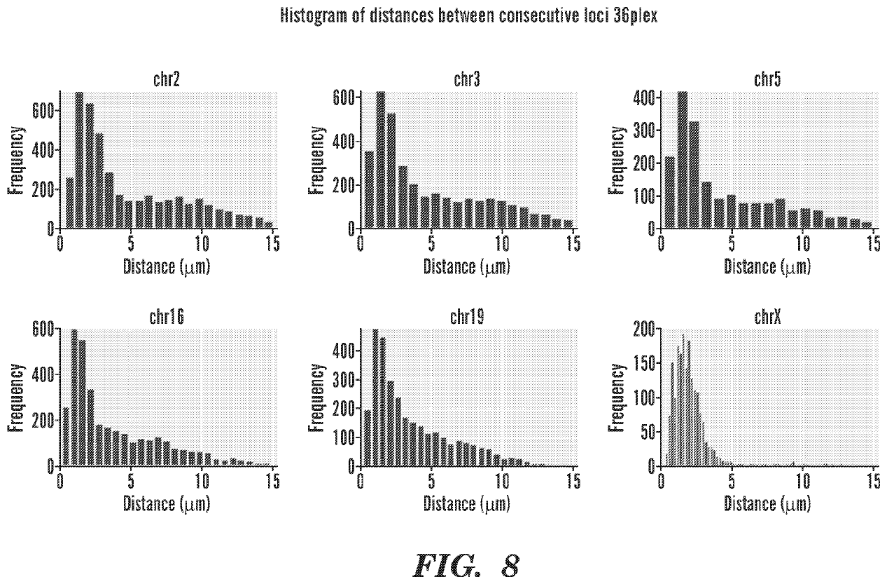

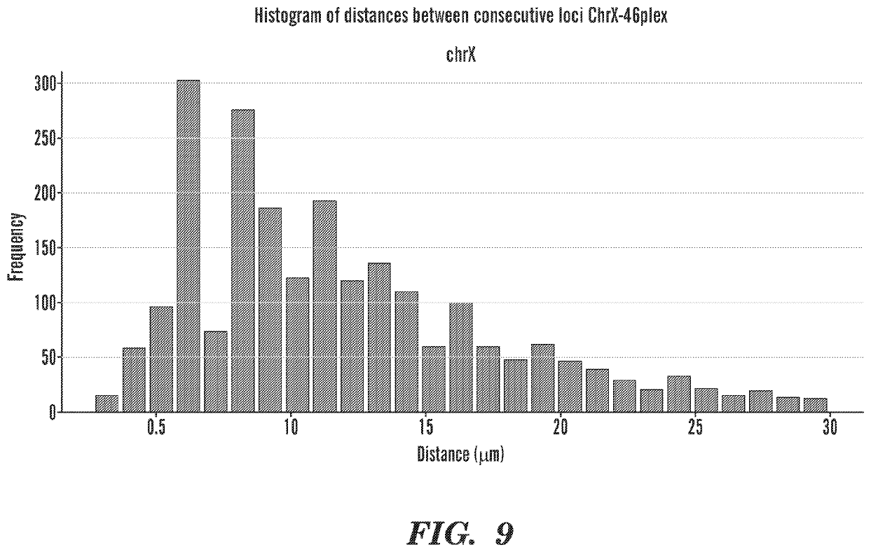

To infer the maximum distance between consecutive loci used in the chromosome tracing the list of detected barcodes for all 36plex datasets was filtered to discard mitotic cells as explained in the Detection efficiency section. Patches formed by one single pixel were also filtered out. After the filtering process the 36plex dataset was composed of 1,171 nuclei and 48,352 barcodes. Then the distances were calculated between consecutive loci for each chromosome in each nucleus. The plots of the histograms of those distances shows the expected bimodal distributions for the chromosomes except for the chromosome X as foreseen from male cells (see e.g., FIG. 8). Bimodality is more evident in bigger chromosomes because those tend to be in the periphery of the nucleus while smaller chromosomes prefer the interior.

After the inspection of the histograms 4 μm was selected as a general maximum distance between consecutive loci and a slightly higher value of 4.5 μm for the chromosome territory.

In the case of ChrX-46plex a similar approach was followed. After the filtering process the ChrX-46plex dataset contained 189 nuclei and 7752 barcodes. Based on the histograms of distances between consecutive loci 2.5 μm was selected as a general maximum distance.

Clustering of 3D Structures for chrX-46Plex

After tier 2 detection 177 cells were used for the chrX-46plex library with an average of 34 detected loci. One of the cells was discarded for having less than 23 identified barcodes as to require at least 50% detection efficiency per cell in all the 3D structures. Then for each chromosome the pairwise distances were calculated between all of their detected targets and those were used as a measure of similarity to build a distance matrix. The coincident distances between structures were used to cluster them hierarchically using the Ward method.

The Calinski-Harabasz criterion for clustering evaluation was used to evaluate the optimal number of clusters.

TABLE 3
Intensity thresholds

| | | | Threshold | | Threshold in |
|---|---|---|---|---|---|
| | Number | | single | Threshold | toto (if |
| Chemistry | of Rounds | Streets | round | average | available) |
| JEB | 5 | MS | 62000 | 61000 | 47000 |
| JEB | 5 | MSBS | 64000 | 63500 | 47000 |
| SOLiD | 4 | MS | 64000 | 63500 | 47000 |
| SOLiD | 4 | MSBS | 64000 | 63500 | 47000 |

Threshold single round: Minimum intensity value at least in one round

Threshold average: Minimum intensity value of the average of all rounds

Threshold in toto: Minimum intensity value of in toto image

### Example 4

### JEB Overhangs

As shown in FIG. 11A-11E, JEB overhang functions to recruit complementary oligonucleotides. Complementary oligonucleotides can be labeled with fluorophores, elemental metals, DNA origami structures, biotin, and/or other chemical moieties. Complementary oligonucleotides can also recruit additional oligonucleotides to amplify signal, such as through branching reactions. JEB overhang can also be used to amplify signal by methods such as hybridization chain reaction (HCR), signal amplification by exchange reaction (SABER), or rolling circle amplification (RCA). JEB overhang can also be used as a template for in situ sequencing. JEB overhang can serve as a docking strand to recruit imager strands for DNA-PAINT (DNA point accumulation in nanoscale topology) to mediate super resolution microscopy; see e.g., Schnitzbauer et al., Super-resolution microscopy with DNA-PAINT, Nature Protocols volume 12, 1198-1228(2017); the contents of which are incorporated herein by reference in its entirety. Combining JEB overhang with DNA-PAINT allows for significantly increased multiplexing and reduction in imaging time.

### Example 5

Karyotyping Using JEB OligoFISSEQ

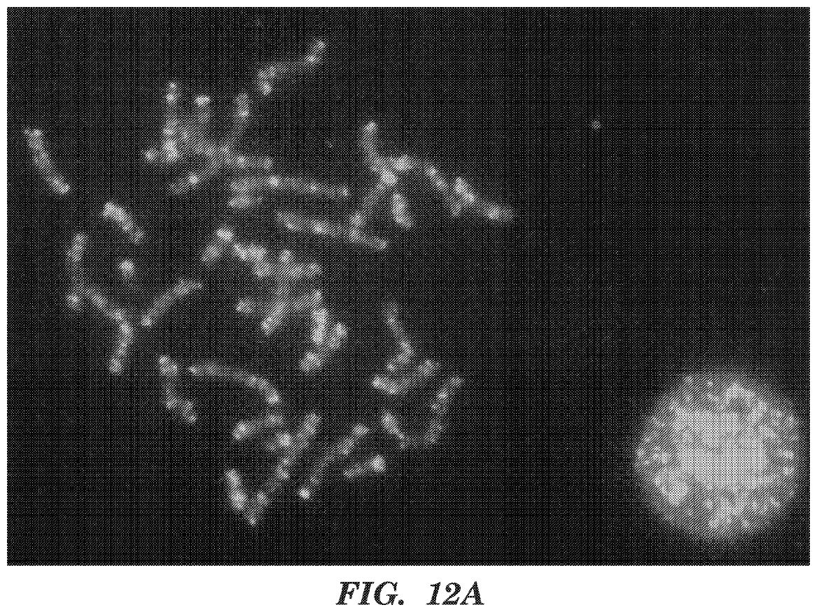

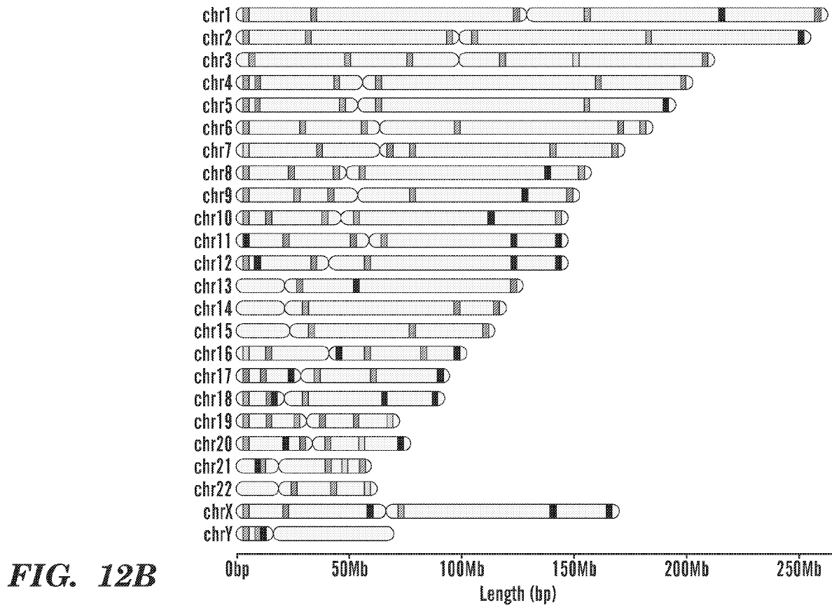

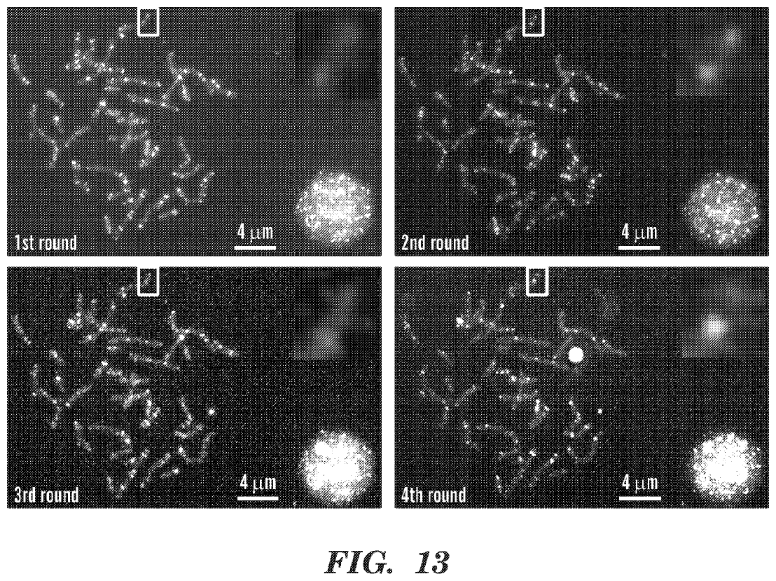

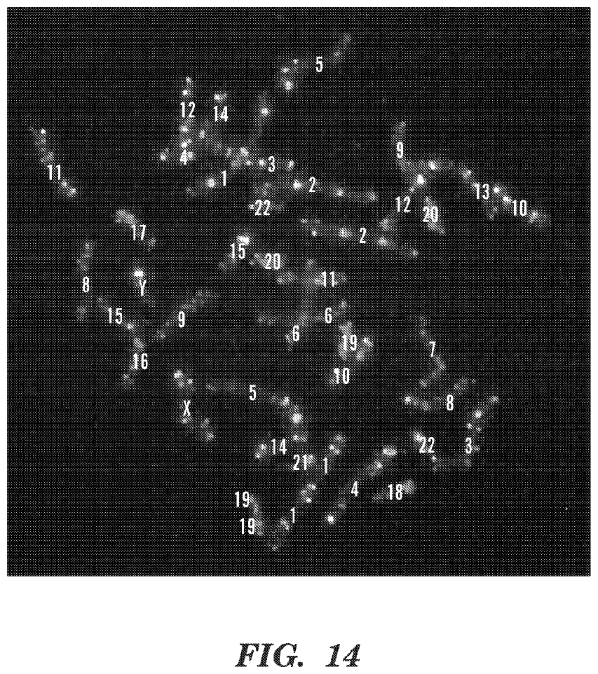

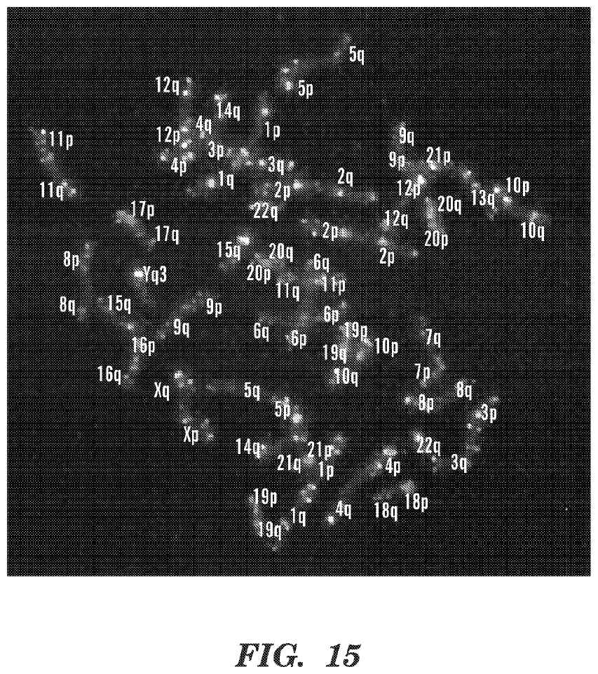

OligoFISSEQ with JEB oligos were used for fast karyotyping of a biological sample. See e.g., FIG. 12A-12B, FIG. 13, FIG. 14, or FIG. 15. The sample can be a human cell nucleus or cell nucleus from any organism. Each chromosome can contain at least 1 or 2 or 3 detectable target(s) per p and q arm. Each chromosome can have at least one detectable target. Metaphase spreads can be obtained from cultured cell nucleus or nucleus extracted from tissue section or organoids or biopsy specimen. Each chromosome arm can contain up to 6, 10, or 20 detectable targets. The target can be an Oligopaint probe. The target can be DNA, RNA or any nucleic acid or chromosomal DNA.

### Example 6

OligoFISSEQ in Presence of Au-NPs

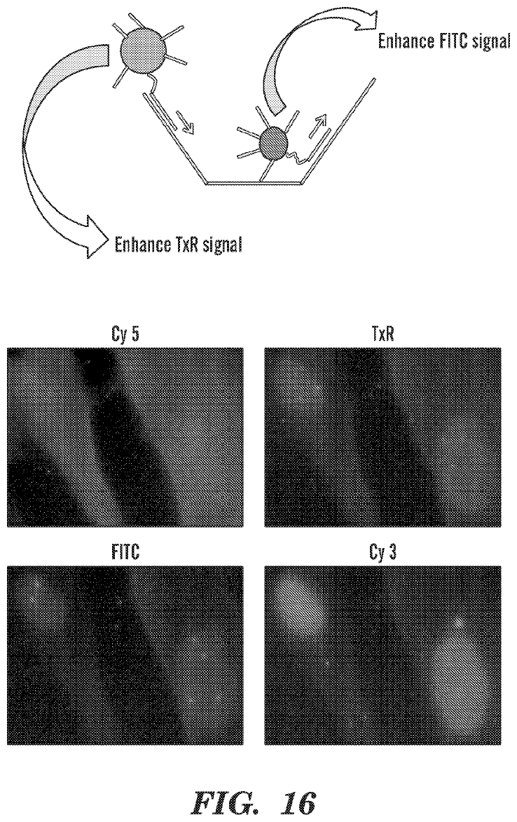

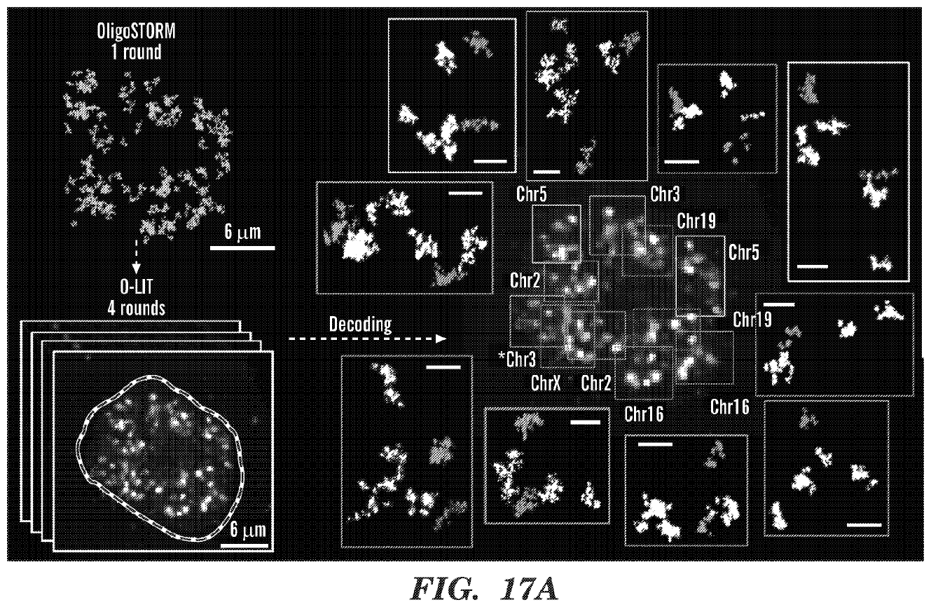

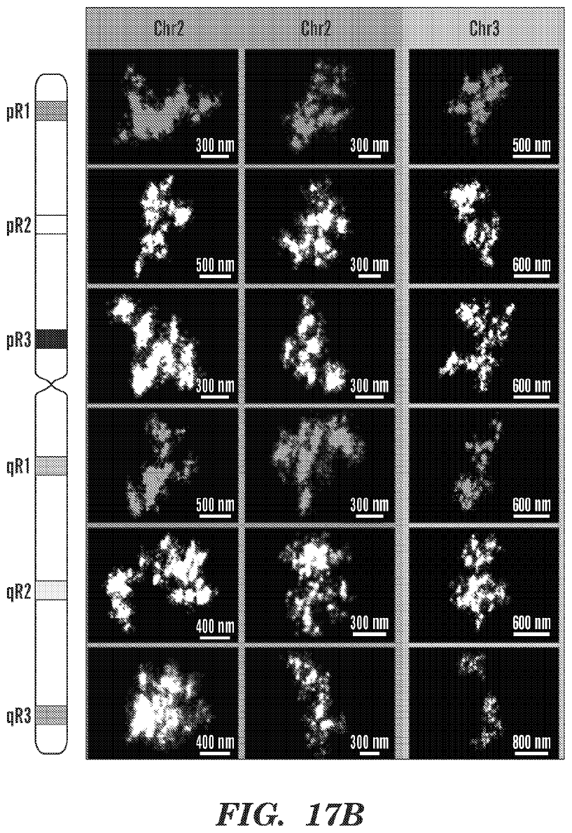

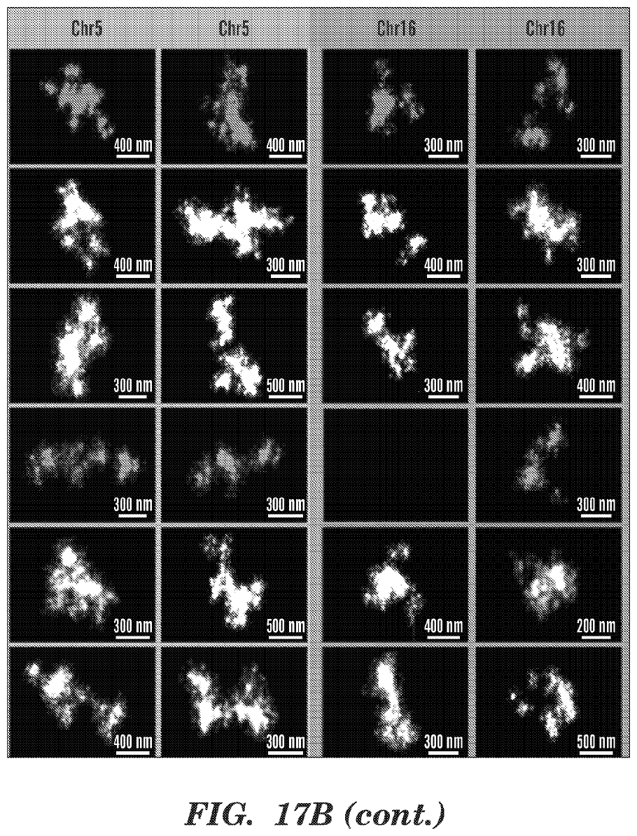

The sequencing primers in one or both the streets can contain gold nanoparticle of diameter 10 nm, 30 nm, 50 nm or any combination of them at the 3′end. The nanoparticle can be gold nanorods. See e.g., FIG. 16. The nanoparticle can be placed at a distance of 30 bp, or greater than 20 bp, from the detectable label. The fluorescence signal of fluorophore-labeled JEB oligos can be enhanced by nanoparticle by at least 1.5 fold, at least 3 fold, at least 10 fold, or at least 50 fold. These nanoparticles can increase the fluorescence signal of detectable labels in the targeted regions in a cell nucleus, or metaphase chromosomes, or metaphase chromosome spreads. The gold nanoparticles labeled targeted regions can be visualized in electron microscopy, or fluorescence microscopy, or dark field microscopy, or any combination thereof. The target can be an Oligopaint probe.

### Example 7

OligoFISSEQ+OligoSTORM

Described herein is a method of detecting and imaging of more than 12 or 66 or 258 or 500 or 5000 targets simultaneously in a biological sample in the super resolution imaging using at least one round of OligoSTORM or two round of OligoSTORM or three round of OligoSTORM or five round of OligoSTORM or 20 rounds of OligoSTORM. See e.g., FIG. 17. All the targets can be visualized at once using fluorophore-labeled oligos complementary to a binding region of the target in one of the streets. At least half of the targets can be visualized at once using fluorophore-labeled oligos complementary to a binding region of the target in one of the streets. The identity of individual targets can then be decoded using 2 or 3 or 5 or 10 or 20 rounds of OligoFISSEQ on the same biological sample. The targets can be at least 1 kb or 15 kb or 50 kb or 100 kb or 1 Mb or whole chromosome or whole genome.

1. A set of at least two readout molecules, each readout molecule comprising:
(a) a 3′ barcode-hybridizing region of nucleotides or analogs thereof, the region comprising a unique sequence distinct from the 3′ region sequence of all other readout molecules in the set;
(b) a 5′ non-barcode-hybridizing region of nucleotides or analogs thereof, the region comprising a sequence identical to the 5′ region sequence of all other readout molecules in the set;
(c) a sulfur modification in place of the bridged oxygen of the phosphate backbone between the 5′ and 3′ regions; and
(d) an optically detectable label.

2. The set of claim 1, wherein the label is a fluorescent label.

3. The set of claim 1, wherein the optically-detectable label comprises or further comprises biotin, amines, metals, metal nanoclusters, noble metal nanoparticles, anchoring molecules, quantum dots, acrydite, or DNA origami structures.

4. The set of claim 1, wherein the label is located at the 5′ end of the readout molecule.

5. The set of claim 1, wherein the set comprises four distinguishable labels.

6. The set of claim 1, wherein the set comprises at least two distinguishable labels.

7.-8. (canceled)

9. The set of claim 1, wherein the readout molecules of each set which comprise a first 3′ region only comprise a first distinguishable label.

10. The set of claim 1, wherein the readout molecules of each set which comprise any selected 3′ region only comprise a corresponding given distinguishable label.

11. The set of claim 1, wherein the 3′ region is at least 1 nucleotide or analog thereof in length.

12. The set of claim 1, wherein the 3′ region is 5 nucleotides or analogs thereof in length.

13. The set of claim 1, wherein the 5′ region comprises only universal nucleotide bases.

14. The set of claim 1, wherein the 5′ region comprises only deoxyinosine nucleotides.

15. The set of claim 1, wherein the 5′ region is at least 1 nucleotide or analog thereof in length.

16. The set of claim 1, wherein the 5′ region is 3 nucleotides or analogs thereof in length.

17. The set of claim 1, wherein the at least two readout molecules comprise DNA and/or RNA.

18. (canceled)

19. The set of claim 1, wherein the at least two readout molecules comprise a polypeptide.

20. A set of at least two readout molecules, each readout molecule comprising:
(a) a 3′ barcode-hybridizing region of nucleotides or analogs thereof, the region comprising a unique sequence distinct from the 3′ region sequence of all other readout molecules in the set;
(b) a 5′ non-barcode-hybridizing region of nucleotides or analogs thereof, and
(c) a sulfur modification in place of the bridged oxygen of the phosphate backbone between the 5′ and 3′ regions.

21.-32. (canceled)

33. A readout molecule comprising:
(a) a 3′ barcode-hybridizing region of nucleotides or analogs thereof, the region comprising a unique sequence distinct from the 3′ region sequence of all other readout molecules in a set of readout molecules;
(b) a 5′ non-barcode-hybridizing region of nucleotides or analogs thereof, the region comprising a sequence identical to the 5′ region sequence of all other readout molecules in the set;
(c) a sulfur modification in place of the bridged oxygen of the phosphate backbone between the 5′ and 3′ regions;
(d) an optically detectable label; and
(e) a nanoparticle.

34.-55. (canceled)

56. A method of detecting at least one target molecule in a sample, the method comprising:
(a) contacting the sample with at least one oligonucleotide tag, each oligonucleotide tag comprising:
(i) a recognition domain that binds specifically to a target molecule to be detected, and
(ii) a street comprising a barcode region that comprises at least one barcode bit;
(b) contacting the sample with a set of readout molecules according to claim 1; and
(c) detecting the relative order of the optically detectable labels hybridized to the at least one oligonucleotide tag, wherein the at least one oligonucleotide tag is hybridized to the at least one target molecule, whereby the relative order of the optically detectable labels permits identification of which oligonucleotide tag is hybridized to the target molecule at that location.

57.-100. (canceled)

101. A method of karyotyping a biological sample, the method comprising:
(a) contacting the sample with at least one oligonucleotide tag specific to at least one chromosome, each oligonucleotide tag comprising:
(i) a recognition domain that binds specifically to a target molecule to be detected, and
(ii) a street comprising a barcode region that comprises at least one barcode bit;
(b) contacting the sample with a set of readout molecules according to claim 1;
(c) detecting the relative order of the optically detectable labels hybridized to the at least one oligonucleotide tag, wherein the at least one oligonucleotide tag is hybridized to the at least one target molecule, whereby the relative order of the optically detectable labels permits identification of which oligonucleotide tag is hybridized to the target molecule at that location; and
d. determining the identity of at least one chromosome according to the identity of the least one oligonucleotide tag specific to the at least one chromosome.

102.-114. (canceled)

115. A method of producing a high resolution image of at least one target molecule in a sample, the method comprising:
(a) imaging the at least one target molecule using at least one round of a high resolution imaging method; and
(b) determining the identity of the at least one imaged target molecule, comprising:
(i)contacting the sample with at least one oligonucleotide tag, each oligonucleotide tag comprising:
(A) a recognition domain that binds specifically to a target molecule to be detected, and
(B) a street comprising a barcode region that comprises at least one barcode bit;
(ii) contacting the sample with a set of readout molecules according to claim 1; and
(iii) detecting the relative order of the optically detectable labels hybridized to the at least one oligonucleotide tag, wherein the at least one oligonucleotide tag is hybridized to the at least one target molecule, whereby the relative order of the optically detectable labels permits identification of which oligonucleotide tag is hybridized to the target molecule at that location.

116.-141. (canceled)
